SEQUENCE LISTING (90 sequences)

SEQ ID NO: 1 - protein, 21 aa, Artificial Sequence
  Synthetic
  misc_feature (1)..(1): Xaa can be any natural or artificial amino acid
  misc_feature (5)..(5): Xaa can be any natural or artificial amino acid
XIFSXLAGKKIKNLLISGLKG

SEQ ID NO: 2 - protein, 21 aa, Artificial Sequence
  Synthetic
  misc_feature (2)..(2): Xaa can be any natural or artificial amino acid
  misc_feature (6)..(6): Xaa can be any natural or artificial amino acid
GXFSKXAGKKIKNLLISGLKG

SEQ ID NO: 3 - protein, 21 aa, Artificial Sequence
  Synthetic
  misc_feature (3)..(3): Xaa can be any natural or artificial amino acid
  misc_feature (7)..(7): Xaa can be any natural or artificial amino acid
GIXSKLXGKKIKNLLISGLKG

SEQ ID NO: 4 - protein, 21 aa, Artificial Sequence
  Synthetic
  misc_feature (4)..(4): Xaa can be any natural or artificial amino acid
  misc_feature (8)..(8): Xaa can be any natural or artificial amino acid
GIFXKLAXKKIKNLLISGLKG

SEQ ID NO: 5 - protein, 21 aa, Artificial Sequence
  Synthetic
  misc_feature (5)..(5): Xaa can be any natural or artificial amino acid
  misc_feature (9)..(9): Xaa can be any natural or artificial amino acid
GIFSXLAGXKIKNLLISGLKG

SEQ ID NO: 6 - protein, 21 aa, Artificial Sequence
  Synthetic
  misc_feature (6)..(6): Xaa can be any natural or artificial amino acid
  misc_feature (10)..(10): Xaa can be any natural or artificial amino acid
GIFSKXAGKXIKNLLISGLKG

SEQ ID NO: 7 - protein, 21 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKG

SEQ ID NO: 8 - protein, 21 aa, Artificial Sequence
  Synthetic
  misc_feature (8)..(8): Xaa can be any natural or artificial amino acid
  misc_feature (12)..(12): Xaa can be any natural or artificial amino acid
GIFSKLAXKKIXNLLISGLKG

SEQ ID NO: 9 - protein, 21 aa, Artificial Sequence
  Synthetic
  misc_feature (9)..(9): Xaa can be any natural or artificial amino acid
  misc_feature (13)..(13): Xaa can be any natural or artificial amino acid
GIFSKLAGXKIKXLLISGLKG

SEQ ID NO: 10 - protein, 21 aa, Artificial Sequence
  Synthetic
  misc_feature (10)..(10): Xaa can be any natural or artificial amino acid
  misc_feature (14)..(14): Xaa can be any natural or artificial amino acid
GIFSKLAGKXIKNXLISGLKG

SEQ ID NO: 11 - protein, 21 aa, Artificial Sequence
  Synthetic
  misc_feature (11)..(11): Xaa can be any natural or artificial amino acid
  misc_feature (15)..(15): Xaa can be any natural or artificial amino acid
GIFSKLAGKKXKNLXISGLKG

SEQ ID NO: 12 - protein, 21 aa, Artificial Sequence
  Synthetic
  misc_feature (12)..(12): Xaa can be any natural or artificial amino acid
  misc_feature (16)..(16): Xaa can be any natural or artificial amino acid
GIFSKLAGKKIXNLLXSGLKG

SEQ ID NO: 13 - protein, 21 aa, Artificial Sequence
  Synthetic
  misc_feature (13)..(13): Xaa can be any natural or artificial amino acid
  misc_feature (17)..(17): Xaa can be any natural or artificial amino acid
GIFSKLAGKKIKXLLIXGLKG

SEQ ID NO: 14 - protein, 21 aa, Artificial Sequence
  Synthetic
  misc_feature (14)..(14): Xaa can be any natural or artificial amino acid
  misc_feature (18)..(18): Xaa can be any natural or artificial amino acid
GIFSKLAGKKIKNXLISXLKG

SEQ ID NO: 15 - protein, 21 aa, Artificial Sequence
  Synthetic
  misc_feature (15)..(15): Xaa can be any natural or artificial amino acid
  misc_feature (19)..(19): Xaa can be any natural or artificial amino acid
GIFSKLAGKKIKNLXISGXKG

SEQ ID NO: 16 - protein, 21 aa, Artificial Sequence
  Synthetic
  misc_feature (16)..(16): Xaa can be any natural or artificial amino acid
  misc_feature (20)..(20): Xaa can be any natural or artificial amino acid
GIFSKLAGKKIKNLLXSGLXG

SEQ ID NO: 17 - protein, 21 aa, Artificial Sequence
  Synthetic
  misc_feature (17)..(17): Xaa can be any natural or artificial amino acid
  misc_feature (21)..(21): Xaa can be any natural or artificial amino acid
GIFSKLAGKKIKNLLIXGLKX

SEQ ID NO: 18 - protein, 21 aa, Artificial Sequence
  Synthetic
  misc_feature (1)..(1): Xaa can be any natural or artificial amino acid
  misc_feature (8)..(8): Xaa can be any natural or artificial amino acid
XIFSKLAXKKIKNLLISGLKG

SEQ ID NO: 19 - protein, 21 aa, Artificial Sequence
  Synthetic
  misc_feature (2)..(2): Xaa can be any natural or artificial amino acid
  misc_feature (9)..(9): Xaa can be any natural or artificial amino acid
GXFSKLAGXKIKNLLISGLKG

SEQ ID NO: 20 - protein, 21 aa, Artificial Sequence
  Synthetic
  misc_feature (3)..(3): Xaa can be any natural or artificial amino acid
  misc_feature (10)..(10): Xaa can be any natural or artificial amino acid
GIXSKLAGKXIKNLLISGLKG

SEQ ID NO: 21 - protein, 21 aa, Artificial Sequence
  Synthetic
  misc_feature (4)..(4): Xaa can be any natural or artificial amino acid
  misc_feature (11)..(11): Xaa can be any natural or artificial amino acid
GIFXKLAGKKXKNLLISGLKG

SEQ ID NO: 22 - protein, 21 aa, Artificial Sequence
  Synthetic
  misc_feature (5)..(5): Xaa can be any natural or artificial amino acid
  misc_feature (12)..(12): Xaa can be any natural or artificial amino acid
GIFSXLAGKKIXNLLISGLKG

SEQ ID NO: 23 - protein, 21 aa, Artificial Sequence
  Synthetic
  misc_feature (6)..(6): Xaa can be any natural or artificial amino acid
  misc_feature (13)..(13): Xaa can be any natural or artificial amino acid
GIFSKXAGKKIKXLLISGLKG

SEQ ID NO: 24 - protein, 21 aa, Artificial Sequence
  Synthetic
  misc_feature (8)..(8): Xaa can be any natural or artificial amino acid
  misc_feature (15)..(15): Xaa can be any natural or artificial amino acid
GIFSKLAXKKIKNLXISGLKG

SEQ ID NO: 25 - protein, 21 aa, Artificial Sequence
  Synthetic
  misc_feature (9)..(9): Xaa can be any natural or artificial amino acid
  misc_feature (16)..(16): Xaa can be any natural or artificial amino acid
GIFSKLAGXKIKNLLXSGLKG

SEQ ID NO: 26 - protein, 21 aa, Artificial Sequence
  Synthetic
  misc_feature (10)..(10): Xaa can be any natural or artificial amino acid
  misc_feature (17)..(17): Xaa can be any natural or artificial amino acid
GIFSKLAGKXIKNLLIXGLKG

SEQ ID NO: 27 - protein, 21 aa, Artificial Sequence
  Synthetic
  misc_feature (11)..(11): Xaa can be any natural or artificial amino acid
  misc_feature (18)..(18): Xaa can be any natural or artificial amino acid
GIFSKLAGKKXKNLLISXLKG

SEQ ID NO: 28 - protein, 21 aa, Artificial Sequence
  Synthetic
  misc_feature (12)..(12): Xaa can be any natural or artificial amino acid
  misc_feature (19)..(19): Xaa can be any natural or artificial amino acid
GIFSKLAGKKIXNLLISGXKG

SEQ ID NO: 29 - protein, 21 aa, Artificial Sequence
  Synthetic
  misc_feature (13)..(13): Xaa can be any natural or artificial amino acid
  misc_feature (20)..(20): Xaa can be any natural or artificial amino acid
GIFSKLAGKKIKXLLISGLXG

SEQ ID NO: 30 - protein, 21 aa, Artificial Sequence
  Synthetic
  misc_feature (14)..(14): Xaa can be any natural or artificial amino acid
  misc_feature (21)..(21): Xaa can be any natural or artificial amino acid
GIFSKLAGKKIKNXLISGLKX

SEQ ID NO: 31 - protein, 21 aa, Artificial Sequence
  Synthetic
  misc_feature (7)..(7): Xaa can be any natural or artificial amino acid
  misc_feature (14)..(14): Xaa can be any natural or artificial amino acid
GIFSKLXGKKIKNXLISGLKG

SEQ ID NO: 32 - protein, 21 aa, Artificial Sequence
  Synthetic
  misc_feature (7)..(7): Xaa can be any natural or artificial amino acid
  misc_feature (11)..(11): Xaa can be any natural or artificial amino acid
  misc_feature (17)..(17): Xaa can be any natural or artificial amino acid
  misc_feature (21)..(21): Xaa can be any natural or artificial amino acid
GIFSKLXGKKXKNLLIXGLKX

SEQ ID NO: 33 - protein, 21 aa, Artificial Sequence
  Synthetic
CIFSCLAGKKIKNLLISGLKG

SEQ ID NO: 34 - protein, 21 aa, Artificial Sequence
  Synthetic
GCFSKCAGKKIKNLLISGLKG

SEQ ID NO: 35 - protein, 21 aa, Artificial Sequence
  Synthetic
GICSKLCGKKIKNLLISGLKG

SEQ ID NO: 36 - protein, 21 aa, Artificial Sequence
  Synthetic
GIFCKLACKKIKNLLISGLKG

SEQ ID NO: 37 - protein, 21 aa, Artificial Sequence
  Synthetic
GIFSCLAGCKIKNLLISGLKG

SEQ ID NO: 38 - protein, 21 aa, Artificial Sequence
  Synthetic
GIFSKCAGKCIKNLLISGLKG

SEQ ID NO: 39 - protein, 21 aa, Artificial Sequence
  Synthetic
GIFSKLCGKKCKNLLISGLKG

SEQ ID NO: 40 - protein, 21 aa, Artificial Sequence
  Synthetic
GIFSKLACKKICNLLISGLKG

SEQ ID NO: 41 - protein, 21 aa, Artificial Sequence
  Synthetic
GIFSKLAGCKIKCLLISGLKG

SEQ ID NO: 42 - protein, 21 aa, Artificial Sequence
  Synthetic
GIFSKLAGKCIKNCLISGLKG

SEQ ID NO: 43 - protein, 21 aa, Artificial Sequence
  Synthetic
GIFSKLAGKKCKNLCISGLKG

SEQ ID NO: 44 - protein, 21 aa, Artificial Sequence
  Synthetic
GIFSKLAGKKICNLLCSGLKG

SEQ ID NO: 45 - protein, 21 aa, Artificial Sequence
  Synthetic
GIFSKLAGKKIKCLLICGLKG

SEQ ID NO: 46 - protein, 21 aa, Artificial Sequence
  Synthetic
GIFSKLAGKKIKNCLISCLKG

SEQ ID NO: 47 - protein, 21 aa, Artificial Sequence
  Synthetic
GIFSKLAGKKIKNLCISGCKG

SEQ ID NO: 48 - protein, 21 aa, Artificial Sequence
  Synthetic
GIFSKLAGKKIKNLLCSGLCG

SEQ ID NO: 49 - protein, 21 aa, Artificial Sequence
  Synthetic
GIFSKLAGKKIKNLLICGLKC

SEQ ID NO: 50 - protein, 21 aa, Artificial Sequence
  Synthetic
CIFSKLACKKIKNLLISGLKG

SEQ ID NO: 51 - protein, 21 aa, Artificial Sequence
  Synthetic
GCFSKLAGCKIKNLLISGLKG

SEQ ID NO: 52 - protein, 21 aa, Artificial Sequence
  Synthetic
GICSKLAGKCIKNLLISGLKG

SEQ ID NO: 53 - protein, 21 aa, Artificial Sequence
  Synthetic
GIFCKLAGKKCKNLLISGLKG

SEQ ID NO: 54 - protein, 21 aa, Artificial Sequence
  Synthetic
GIFSCLAGKKICNLLISGLKG

SEQ ID NO: 55 - protein, 21 aa, Artificial Sequence
  Synthetic
GIFSKCAGKKIKCLLISGLKG

SEQ ID NO: 56 - protein, 21 aa, Artificial Sequence
  Synthetic
GIFSKLACKKIKNLCISGLKG

SEQ ID NO: 57 - protein, 21 aa, Artificial Sequence
  Synthetic
GIFSKLAGCKIKNLLCSGLKG

SEQ ID NO: 58 - protein, 21 aa, Artificial Sequence
  Synthetic
GIFSKLAGKCIKNLLICGLKG

SEQ ID NO: 59 - protein, 21 aa, Artificial Sequence
  Synthetic
GIFSKLAGKKCKNLLISCLKG

SEQ ID NO: 60 - protein, 21 aa, Artificial Sequence
  Synthetic
GIFSKLAGKKICNLLISGCKG

SEQ ID NO: 61 - protein, 21 aa, Artificial Sequence
  Synthetic
GIFSKLAGKKIKCLLISGLCG

SEQ ID NO: 62 - protein, 21 aa, Artificial Sequence
  Synthetic
GIFSKLAGKKIKNCLISGLKC

SEQ ID NO: 63 - protein, 21 aa, Artificial Sequence
  Synthetic
GIFSKLCGKKIKNCLISGLKG

SEQ ID NO: 64 - protein, 21 aa, Artificial Sequence
  Synthetic
GIFSKLCGKKCKNLLICGLKC

SEQ ID NO: 65 - protein, 24 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKGGGG

SEQ ID NO: 66 - protein, 24 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKGGGK

SEQ ID NO: 67 - protein, 23 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKGAG

SEQ ID NO: 68 - protein, 23 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKGAK

SEQ ID NO: 69 - protein, 21 aa, Artificial Sequence
  Synthetic
  misc_feature (7)..(7): Xaa can be any natural or artificial amino acid
  misc_feature (11)..(11): Xaa can be any natural or artificial amino acid
GIFSKLXAKKXKNLLISGLKG

SEQ ID NO: 70 - protein, 21 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISALKG

SEQ ID NO: 71 - protein, 21 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKK

SEQ ID NO: 72 - protein, 6 aa, Artificial Sequence
  Synthetic
  misc_feature (2)..(2): Xaa can be any natural or artificial amino acid
  misc_feature (6)..(6): Xaa can be any natural or artificial amino acid
GXGGGX

SEQ ID NO: 73 - protein, 46 aa, Unknown
  frog Esculentin-1A
GIFSKLAGKKIKNLLISGLKNVGKEVGMDVVRTGIDIAGCKIKGEC

SEQ ID NO: 74 - protein, 23 aa, Artificial Sequence
  Synthetic
  misc_feature (7)..(7): Xaa can be any natural or artificial amino acid
  misc_feature (11)..(11): Xaa can be any natural or artificial amino acid
GIFSKLXGKKXKNLLISGLKGGG

SEQ ID NO: 75 - protein, 21 aa, Artificial Sequence
  Synthetic
GIFSKLAGKKIKNLLISGLKN

SEQ ID NO: 76 - protein, 21 aa, Artificial Sequence
  Synthetic
GIFSKLAGKKIKNLLISGLKG

SEQ ID NO: 77 - protein, 21 aa, Artificial Sequence
  Synthetic
  misc_feature (7)..(7): Xaa can be any natural or artificial amino acid
  misc_feature (11)..(11): Xaa can be any natural or artificial amino acid
GIFSKLXAKKXKNLLISALKG

SEQ ID NO: 78 - protein, 21 aa, Artificial Sequence
  Synthetic
  MISC_FEATURE (1)..(1): Xaa is L-propargyl glycine
  misc_feature (6)..(6): Xaa can be any natural or artificial amino acid
  misc_feature (10)..(10): Xaa can be any natural or artificial amino acid
XIFSKXAGKXIKNLLISGLKG

SEQ ID NO: 79 - protein, 24 aa, Artificial Sequence
  Synthetic
GIFSKLCGKKCKNLLISGLKGGGG

SEQ ID NO: 80 - protein, 24 aa, Artificial Sequence
  Synthetic
GIFSKLCGKKCKNLLISGLKGGGK

SEQ ID NO: 81 - protein, 23 aa, Artificial Sequence
  Synthetic
GIFSKLCGKKCKNLLISGLKGAG

SEQ ID NO: 82 - protein, 23 aa, Artificial Sequence
  Synthetic
GIFSKLCGKKCKNLLISGLKGGG

SEQ ID NO: 83 - protein, 23 aa, Artificial Sequence
  Synthetic
GIFSKLCGKKCKNLLISGLKGAK

SEQ ID NO: 84 - protein, 21 aa, Artificial Sequence
  Synthetic
GIFSKLCAKKCKNLLISGLKG

SEQ ID NO: 85 - protein, 21 aa, Artificial Sequence
  Synthetic
GIFSKLCGKKCKNLLISALKG

SEQ ID NO: 86 - protein, 21 aa, Artificial Sequence
  Synthetic
GIFSKLCAKKCKNLLISALKG

SEQ ID NO: 87 - protein, 21 aa, Artificial Sequence
  Synthetic
  MISC_FEATURE (1)..(1): Xaa is L-propargyl glycine
XIFSKCAGKCIKNLLISGLKG

SEQ ID NO: 88 - protein, 4 aa, Artificial Sequence
  Synthetic
GGGE

SEQ ID NO: 89 - protein, 4 aa, Artificial Sequence
  Synthetic
GGEE

SEQ ID NO: 90 - protein, 6 aa, Artificial Sequence
  Synthetic
GGSGGS

(19) United States
(12) Patent Application Publication
(10) Pub. No.: US 2024/0391965 A1
(43) Pub. Date: Nov. 28, 2024

(54) STAPLED ANTIMICROBIAL PEPTIDES (STAMPS) AND USES THEREOF

(71) Applicant: Lytica Therapeutics, Inc., Boston, MA (US)

(72) Inventors: Rida Mourtada, Boston, MA (US); Daniel T. Cohen, Boston, MA (US)

(73) Assignee: Lytica Therapeutics, Inc., Boston, MA (US)

(21) Appl. No.: 18/289,103

(22) PCT Filed: May 6, 2022
(86) PCT No.: PCT/US2022/028010

§ 371 Date: Oct. 31, 2023

Related U.S. Application Data
(60) Provisional application No. 63/185,673, filed on May 7, 2021.

(51) Int. Cl. C07K 14/46; A61K 38/00; A61K 47/60; A61P 31/04

(52) U.S. Cl. CPC C07K 14/463; A61K 47/60; A61P 31/04; A61K 38/00

(57) ABSTRACT

Provided are stapled antimicrobial peptides (i.e., StAMPs), including cysteine-stapled antimicrobial peptides (i.e., C-StAMPs), and methods of using the same (e.g., for treating bacterial infections caused by Gram-negative bacteria). In certain embodiments, the stapled peptides are based on the amino acid sequence of the antimicrobial peptide Esculentin-1A and comprise one or more dithio staples. Also provided are unstapled peptides which can serve as synthetic precursors to the stapled peptides provided herein.

### RELATED APPLICATIONS

This application claims priority under 35 U.S.C. 119(e) to U.S. Provisional patent application, U.S. Ser. No. 63/185,673, filed May 7, 2021, the entire contents of which is incorporated herein by reference.

### GOVERNMENT SUPPORT

This invention was made with government support under 6 IDSEP160030-04-01, awarded by the Department of Health and Human Services. The government has certain rights in the invention.

### BACKGROUND

The rise in antibiotic resistance across the globe poses a major threat to human health. As the number of newly approved antibiotics has decreased over the past decade, the need for new agents to combat drug-resistant microbes has greatly increased. Because the evolution of resistance in bacteria has outpaced modern drug development, there is a need for new agents that overcome antibiotic-resistance, especially in Gram-negative bacteria.

Antimicrobial peptides (AMPs) are a subclass of natural peptides expressed by diverse species of life to combat infections, predominantly of the skin or localized compartments. See, e.g., Brogden, K. A., Nat. Rev. Microbiol. 3, 238-250 (2005); Brown, K. L. et al. Curr. Opin. Immunol. 18, 24-30 (2006); Hancock, R. E. et al. Nat. Biotechnol. 24, 1551-1557 (2006). Cationic AMPs kill bacteria by membrane lysis, a mechanism significantly less prone to inducing antibiotic resistance. See, e.g., Fjell, C. D. et al. Nat. Rev. Drug Discov. 11, 37-51 (2012).

Indeed, AMPs have been proposed as a potential solution to the global threat of multidrug resistance; however, linear AMPs can be structurally unstable, proteolytically labile, and/or cause nonspecific membrane toxicity that has largely precluded their clinical translation for internal use. See, e.g., Theuretzbacher, U. Curr. Opin. Microbiol. 39, 106-112 (2017); Woodford, N. et al. J. Antimicrob. Chemother. 63, 225-229 (2009); Blair, J. M. et al. Nat. Rev. Microbiol. 13, 42-51 (2015). Since their discovery, three decades of groundbreaking research have elucidated the properties of natural and non-natural AMPs, but the insights have not yielded any AMP-based antibiotic drugs as clinical candidates. See, e.g., Steiner, H. et al. Nature 292, 246-248 (1981).

One approach to reducing the proteolytic lability of peptides in vivo involves enforcing an α-helical structure by inserting all-hydrocarbon staples. See, e.g., Bird, G. H. et al. Proc. Natl Acad. Sci. USA 107, 14093-14098 (2010); Schafmeister, C. E. et al. JACS 122, 5891-5892 (2000); Walensky, L. D. et al. Science 305, 1466-1470 (2004). All-hydrocarbon stapling has been applied to maximize the utility of bioactive helices for targeting protein interactions, with the first clinical-grade anticancer stapled peptide in phase I and II trials. See, e.g., Bird, G. H. et al. Nat. Chem. Biol. 12, 845-852 (2016); Walensky, L. D. et al. J. Med. Chem. 57, 6275-6288 (2014); Bird, G. H. et al. J. Clin. Invest. 124, 2113-2124 (2014); Meric-Bernstam, F. et al. J. Clin. Oncol. 35, 2505 (2017).

Attempts to install staples into AMP sequences have in some instances yielded constructs with improved α-helicity and proteolytic resistance but variable antimicrobial activity and indiscriminate membrane lysis, reflective of a low therapeutic index. See, e.g., Chapuis, H. et. al. Amino Acids 43, 2047-2058 (2012); Dinh, T. T. et al. Bioorg. Med. Chem. Lett. 25, 4016-4019 (2015); Migon, D. et al. Protein J. 37, 2-12 (2018); Pham, T. K. et al. Bioorg. Med. Chem. Lett. 23, 6717-6720 (2013); Stone, T. A. et al. Bioorg. Med. Chem. 26, 1189-1196 (2017); Stone, T. A. et al. Bioorg. Med. Chem. 26, 1189-1196 (2017). Thus, stapling has in some instances been viewed as contributing to nonspecific AMP toxicity.

### SUMMARY OF THE INVENTION

There is currently a need for new antimicrobials with high antimicrobial activity and low nonspecific toxicity such as stapled antimicrobial peptides. Examples of stapled antimicrobial peptides (StAMPs) and methods of using the same, can be found in, e.g., International PCT application Publication Nos. WO 2017/004591, published Jan. 5, 2017; and WO 2019/018499, published Jan. 24, 2019, the entire contents of each of which are incorporated herein by reference. In one aspect, provided herein are StAMPs based on the sequence of Esculentin-1A that may contain one or more dithio staples.

The present disclosure relates in part to new stapled antimicrobial peptides (StAMPs) based on the sequence of Esculentin-1A which can be used to treat and/or prevent infectious diseases (e.g., bacterial infections). In certain embodiments, the placement of one or more staples within the peptide may confer advantageous properties (e.g., improved antimicrobial activity, selectivity for killing Gram-negative bacteria, and/or reduced toxicity) and/or allow for certain stapling technology to be used (e.g., thiol-stapling). Peptides provided herein may be selectively cytotoxic to microbial cells (e.g., bacterial cells) over mammalian cells. In certain embodiments, this selectivity leads to reduced toxicity (e.g., reduced renal or hepatic toxicity) in a subject (e.g., human).

One aspect of the present disclosure relates to StAMPs based on the sequence of Esculentin-1A that are singly stapled, i.e., comprising exactly one internal crosslink. For instance, provided herein are peptides, and pharmaceutically acceptable salts thereof, comprising 10 or more contiguous amino acids of Esculentin-1A (SEQ ID NO: 73) and comprising exactly one internal crosslink connecting two crosslinked amino acids, wherein:
two amino acids of the 10 or more contiguous amino acids of Esculentin-1A are independently substituted by X¹ and X²; and
X¹ and X² are independently the crosslinked amino acids;
optionally wherein the amino acid sequence includes 1 to 3 amino acid substitutions, inclusive, at positions other than X¹ and X².

In certain embodiments, the peptide comprises 20 or more contiguous amino acids of Esculentin-1A (SEQ ID NO: 73); optionally wherein the amino acid sequence includes 1 to 6 amino acid substitutions, inclusive, at positions other than X¹ and X².

In certain embodiments, X¹ and X² are separated by 3 amino acids, forming an “i+4 crosslink.” In certain embodiments, X¹ and X² are separated by 6 amino acids, forming an “i+7 crosslink.”

For example, in certain embodiments, a stapled (i.e., crosslinked) peptide provided herein comprises the following amino acid sequence:

(SEQ ID NO: 7)
G I F S K L X¹ G K K X² K N L L I S G L K G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X². In certain embodiments, X¹ and X² are connected via a dithio crosslink (e.g., (mxy), (pxy), (but), (bbn), (bbf), (bbp), (pfb), (hfb)).

Another aspect of the present disclosure relates to StAMPs based on the amino acid sequence of Esculentin-1A that include one or more dithio staples (i.e., dithio crosslinks). Provided herein are peptides, and pharmaceutically acceptable salts thereof, comprising 10 or more contiguous amino acids of Esculentin-1A (SEQ ID NO: 73) and at least one internal dithio crosslink, wherein:
at least 2 amino acids of the 10 or more contiguous amino acids of Esculentin-1A are substituted by a pair of dithio-crosslinked amino acids;
optionally wherein the amino acid sequence includes 1 to 3 additional amino acid substitutions, inclusive (i.e., at positions other than the dithio-crosslinked amino acids).

In certain embodiments, the peptide comprises 20 or more contiguous amino acids of Esculentin-1A; optionally wherein the amino acid sequence includes 1 to 6 additional amino acid substitutions, inclusive.

As described herein, the peptide may comprise one or more (e.g., 1, 2) internal dithio crosslinks. In certain embodiments, 2 amino acids of the 10 or more (e.g., 20 or more) contiguous amino acids of Esculentin-1A (SEQ ID NO: 73) are substituted by a pair of dithio-crosslinked amino acids, thereby forming one dithio crosslink. In certain embodiments, 4 amino acids of the 10 or more (e.g., 20 or more) contiguous amino acids of Esculentin-1A (SEQ ID NO: 73) are independently substituted by two pairs of dithio-crosslinked amino acids, thereby forming two dithio crosslinks.

In certain embodiments, at least one pair of dithio-crosslinked amino acids are separated by 3 amino acids, forming an “i+4 dithio crosslink.” In certain embodiments, at least one pair of dithio-crosslinked amino acids are separated by 6 amino acids, forming an “i+7 dithio crosslink.”

In certain embodiments, a dithio stapled (i.e., dithio crosslinked) peptide provided herein comprises the following amino acid sequence:

(SEQ ID NO: 7)
G I F S K L X¹ G K K X² K N L L I S G L K G,

or a pharmaceutically acceptable salt thereof, wherein:
X¹ and X² are independently amino acids connected via a dithio crosslink;
optionally wherein the amino acid sequence includes 1 to 6 amino acid substitutions, inclusive, at positions other than X¹ and X².

In certain embodiments, X¹ and X² in SEQ ID NO: 7 are connected via a dithio crosslink selected from (mxy), (pxy), and (but).

For example, in certain embodiments, a dithio-stapled (i.e., dithio-crosslinked) peptide or pharmaceutically acceptable salt thereof comprising SEQ ID NO: 7 is one of the following peptides (the C-terminus of each of StAMPs 2-4 is amidated with —NH₂):

| | | | | | | | | | | | | | | | | | | | | | | SEQ | X¹-X² |
|---|---|---|---|---|---|---|---|---|---|---|---|---|---|---|---|---|---|---|---|---|---|---|---|
| StAMP | | | | | | | | | | | | | | | | | | | | | | ID | Cross- |
| # | Sequence | | | | | | | | | | | | | | | | | | | | | NO: | link |
| 2 | G | I | F | S | K | L | X¹ | G | K | K | X² | K | N | L | L | I | S | G | L | K | G | 7 | mxy |
| 3 | G | I | F | S | K | L | X¹ | G | K | K | X² | K | N | L | L | I | S | G | L | K | G | 7 | pxy |
| 4 | G | I | F | S | K | L | X¹ | G | K | K | X² | K | N | L | L | I | S | G | L | K | G | 7 | but |

The amino acid sequences and peptides provided herein may comprise one or more additional modifications described herein, such as amino acid deletions, amino acid additions, C-terminus modifications, etc.

In another aspect, provided herein are pharmaceutical compositions comprising a peptide provided herein, or a pharmaceutically acceptable salt thereof, and one or more pharmaceutically acceptable carriers or excipients. In certain embodiments, a pharmaceutical composition provided herein comprises a therapeutically and/or prophylactically effective amount of a peptide provided herein. The pharmaceutical compositions described herein are useful for treating and/or preventing diseases (e.g., bacterial infections) in a subject.

In other aspects, provided herein are methods and uses of the compounds and pharmaceutical compositions provided herein, including the following:
(a) Methods of treating and/or preventing an infectious disease (e.g., bacterial infection) in a subject comprising administering to the subject a therapeutically and/or prophylactically effective amount of a peptide (e.g., StAMP) provided herein, or a pharmaceutically acceptable salt thereof, or a pharmaceutical composition thereof. In certain embodiments, the bacterial infection is caused by Gram-negative bacteria. In certain embodiments, the bacterial infection is caused by antibiotic-resistant bacteria (e.g., antibiotic-resistant Gram-negative bacteria).
(b) Methods of killing bacteria and/or inhibiting the growth of bacteria (e.g., in vivo or in vitro) with a peptide (e.g., StAMP) provided herein, or a pharmaceutically acceptable salt thereof, or a pharmaceutical composition thereof. In certain embodiments, the bacteria are Gram-negative bacteria. In certain embodiments, the bacteria are antibiotic-resistant Gram-negative bacteria. In certain embodiments, the bacteria are Escherichia coli (E. coli), Acinetobacter baumannii (A. baumannii), Pseudomonas aeruginosa (P. aeruginosa), or Klebsiella pneumoniae (K. pneumoniae). In certain embodiments, the killing and/or inhibiting is selective for Gram-negative over Gram-positive bacteria.
(c) Methods of selectively killing microbial cells and/or inhibiting the growth of microbial cells (e.g., bacterial cells) over mammalian cells comprising contacting the microbial (e.g., bacterial) and mammalian cells with a peptide (e.g., StAMP) provided herein, or a pharmaceutically acceptable salt thereof, or a pharmaceutical composition thereof.
(d) Methods of selectively lysing microbial cells (e.g., bacterial cells) over mammalian cells comprising contacting the microbial (e.g., bacterial) and mammalian cells with a peptide (e.g., StAMP) provided herein, or a pharmaceutically acceptable salt thereof, or a pharmaceutical composition thereof.

Also provided herein are peptides (e.g., StAMPs) described herein, and pharmaceutically acceptable salts thereof, and pharmaceutical compositions thereof, for use in any of the methods described herein (e.g., for treating an infectious disease in a subject, treating a bacterial infection in a subject, killing and/or inhibiting the growth of bacteria in a subject, selectively killing and/or inhibiting the growth of bacterial cells over mammalian cells in a subject, etc.).

In another aspect, also provided herein are uses of peptides (e.g., StAMPs) described herein, and pharmaceutically acceptable salts thereof, and pharmaceutical compositions thereof, for the manufacture of medicament (e.g., for treating an infectious disease in a subject, treating a bacterial infection in a subject, killing and/or inhibiting the growth of bacteria in a subject, selectively killing and/or inhibiting the growth of bacterial cells over mammalian cells in a subject, etc.).

In another aspect, provided herein are kits comprising a peptide (e.g., StAMP) provided herein, or a pharmaceutically acceptable salt thereof, or a pharmaceutical composition thereof. The kits described herein may include a single dose or multiple doses of the peptide or pharmaceutical composition thereof. The kits described herein are useful in any method or use provided herein, and optionally further comprise instructions for using the kit (e.g., instructions for using the peptide or composition included in the kit).

In yet another aspect, provided herein are unstapled or partially stapled peptides which can serve as synthetic precursors to the stapled peptides provided herein. Such unstapled peptides comprise pairs of amino acids comprising reactive moieties (e.g., thiols) capable of forming crosslinks with one another. Also provided herein are methods of preparing stapled peptides provided herein from said unstapled peptides provided herein.

For example, the unstapled (i.e., uncrosslinked) peptide may include thiol-containing amino acids that can undergo thiol-stapling reactions to form one or more internal dithio crosslinks. “Thiol-stapling” involves reacting two thiol moieties in the presence of a crosslinking reagent (e.g., a dihalide) to form the crosslink. Examples of thiol-stapling reactions can be found in, e.g., US Patent Application Publication No. US 2019/0382443 A1, published Dec. 19, 2019; U.S. Pat. No. 9,670,484 B2, issued Jun. 6, 2017; U.S. Pat. No. 9,644,201 B2, issued May 9, 2017; US Patent Application Publication No. 2017/0067045 A1, published Mar. 9, 2017; Peraro, L. et al. Methods in Enzymology vol. 580 (2016): 303-32; and Kale, S. S. et al. Nature Chem 10, 715-723 (2018), the entire contents of each of which is incorporated herein by reference.

A general scheme of a thiol-stapling reaction is shown below in Scheme 1, wherein LG is a leaving group (e.g., halogen, such as bromine), and L² is as described herein.

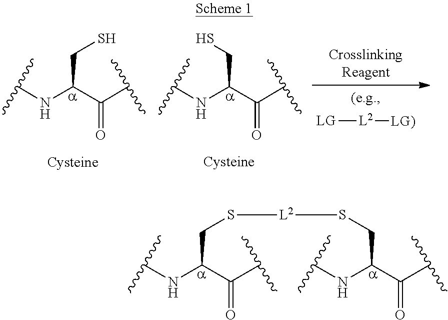

Provided herein are unstapled (i.e., uncrosslinked) peptides comprising 10 or more (e.g., 20 or more) contiguous amino acids of Esculentin-1A (SEQ ID NO: 73); wherein:
2 or 4 amino acids of the 10 or more (e.g., 20 or more) contiguous amino acids of Esculentin-1A are independently substituted by thiol-containing amino acids (e.g., cysteine (C)); and
optionally wherein the amino acid sequence includes additional amino acid substitutions (e.g., 1 to 3, or 1 to 6, additional amino acid substitutions, inclusive).

In certain embodiments, 2 amino acids of the 10 or more (e.g., 20 or more) contiguous amino acids of Esculentin-1A are each substituted by cysteines (C). In certain embodiments, 4 amino acids of the 10 or more (e.g., 20 or more) contiguous amino acids of Esculentin-1A are each substituted by cysteines (C). In certain embodiments, at least two C are separated by 3 amino acids. In certain embodiments, at least two C are separated by 6 amino acids.

For example, in certain embodiments, an unstapled (i.e., uncrosslinked) peptide provided herein comprises the following amino acid sequence:

(SEQ ID NO: 39)
G I F S K L C G K K C K N L L I S G L K G,

or a pharmaceutically acceptable salt thereof.

The details of certain embodiments of the invention are set forth in the Detailed Description of Certain Embodiments, as described below. Other features, objects, and advantages of the invention will be apparent from the Definitions, Examples, and Claims.

### Definitions

### General Definitions

The following definitions are general terms used throughout the present application.

The terms “peptide” and “polypeptide” are used interchangeably and refer to a polymer of amino acid residues linked together by peptide bonds. The terms also include proteins, and refer to peptides, polypeptides, and proteins, of any size, structure, or function. Typically, a peptide will be at least three amino acids long, or at least the length required by an amino acid sequence provided herein. A peptide may refer to an individual peptide or a collection of peptides. Peptides provided herein can include natural amino acids and/or unnatural amino acids (i.e., compounds that do not occur in nature but that can be incorporated into a peptide chain) in any combination. One or more of the amino acids in a peptide may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A peptide may be a fragment or modified version of a naturally occurring peptide or protein. A peptide may be naturally occurring, recombinantly expressed, synthetic, or any combination of these.

A peptide provided herein can be of any length. In certain embodiments, a peptide is 100 amino acids or fewer in length. In certain embodiments, a peptide is 90 amino acids or fewer in length. In certain embodiments, a peptide is 80 amino acids or fewer in length. In certain embodiments, a peptide is 70 amino acids or fewer in length. In certain embodiments, a peptide is 60 amino acids or fewer in length. In certain embodiments, a peptide is 50 amino acids or fewer in length. In certain embodiments, a peptide is 45 amino acids or fewer in length. In certain embodiments, a peptide is 40 amino acids or fewer in length. In certain embodiments, a peptide is 35 amino acids or fewer in length. In certain embodiments, a peptide is 30 amino acids or fewer in length. In certain embodiments, a peptide is 25 amino acids or fewer in length. In certain embodiments, a peptide is 20 amino acids or fewer in length. In certain embodiments, a peptide is 19, 20, 21, 22, 23, 24, 25, 26, 27, 28, 29, 30, 31, 32, 33, 34, 35, 36, 37, 38, 39, or 40 amino acids or fewer in length. In certain embodiments, a peptide is at least the length of an amino acid sequence provided herein.

The term “amino acid” refers to a molecule containing both an amino group and a carboxyl group. Amino acids include alpha-amino acids, the generic structure of which is depicted below. Each amino acid referred to herein may be denoted by a 1- to 4-letter code (e.g., R and Arg represent L-Arginine, hArg represents L-homoarginine).

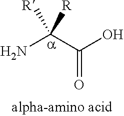

Suitable amino acids include, without limitation, natural alpha-amino acids such as D- and L-isomers of the 20 common naturally occurring alpha-amino acids found in peptides (e.g., A, R, N, C, D, Q, E, G, H, I, L, K, M, F, P, S, T, W, Y, V, as provided below), and unnatural alpha-amino acids.

Exemplary natural alpha-amino acids (with one-letter code provided in parentheses) include L—alanine (A), L—arginine (R), L—asparagine (N), L—aspartic acid (D), L—cysteine (C), L—glutamic acid (E), L—glutamine (Q), glycine (G), L—histidine (H), L—isoleucine (I), L—leucine (L), L—lysine (K), L—methionine (M), L—phenylalanine (F), L—proline (P), L—serine (S), L—threonine (T), L—tryptophan (W), L—tyrosine (Y), and L—valine (V).

Exemplary unnatural alpha-amino acids include D—arginine, D—asparagine, D—aspartic acid, D—cysteine, D—glutamic acid, D—glutamine, D—histidine, D—isoleucine, D—leucine, D—lysine, D—methionine, D—phenylalanine, D—proline, D—serine, D—threonine, D—tryptophan, D—tyrosine, D—valine, Di-vinyl, α-methyl-alanine (Aib), α-methyl-arginine, α-methyl-asparagine, α-methyl-aspartic acid, α-methyl-cysteine, α-methyl-glutamic acid, α-methyl-glutamine, α-methyl-histidine, α-methyl-isoleucine, α-methyl-leucine, α-methyl-lysine, α-methyl-methionine, α-methyl-phenylalanine, α-methyl-proline, α-methyl-serine, α-methyl-threonine, α-methyl-tryptophan, α-methyl-tyrosine, α-methyl-valine, norleucine, and terminally unsaturated alpha-amino acids. There are many known unnatural amino acids any of which may be included in the peptides of the present disclosure. See for example, S. Hunt, The Non-Protein Amino Acids: In Chemistry and Biochemistry of the Amino Acids, edited by G. C. Barrett, Chapman and Hall, 1985. Unnatural amino acids also include amino acids comprising nitrogen substitutents.

Certain amino acids referred to herein are provided in Table 1 below (represented by name, structure, and 1- to 4-letter code).

TABLE 1
Certain Amino Acids

| Name | Code | Structure |
|---|---|---|
| L-Norleucine | B | 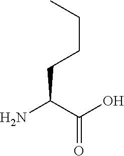 |
| L-Ornithine | Orn | 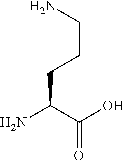 |
| L-Diaminobutyric Acid | Dab | 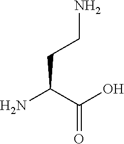 |
| L-Diamino- propionic Acid | Dap | 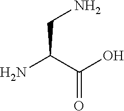 |
| β-Alanine | βAla | 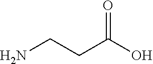 |
| L-Homoarginine | hArg | 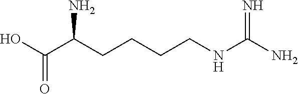 [SMILES: N=C(N)NCCCC[C@H](N)C(=O)O] |
| D-Phenylalanine | F¹ | 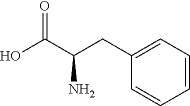 [SMILES: N[C@H](Cc1ccccc1)C(=O)O] |
| L-4- trifluoromethyl phenylalanine | F² | 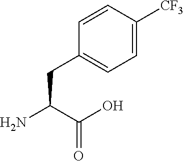 [SMILES: N[C@@H](Cc1ccc(C(F)(F)F)cc1)C(=O)O] |
| L-4-t-butyl phenylalanine | F³ | 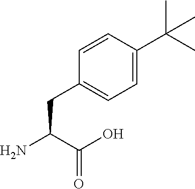 [SMILES: CC(C)(C)c1ccc(C[C@H](N)C(=O)O)cc1] |
| L-4-fluoro phenylalanine | F⁴ | 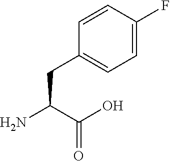 [SMILES: N[C@@H](Cc1ccc(F)cc1)C(=O)O] |
| L-4-methyl phenylalanine | F⁵ | 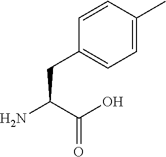 |
| S-2-(4′-pentenyl) alanine | S⁵ | 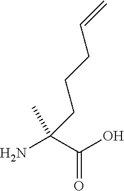 |
| R-2-(7′-octenyl) alanine | R⁸ | 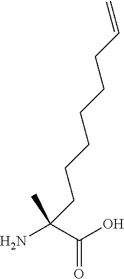 |
| L-propargyl glycine | J | 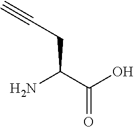 |
| L-Azidolysine | Azi | 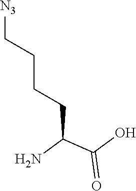 [SMILES: [N-]=[N+]=NCCCC[C@H](N)C(=O)O] |

The term “amino acid substitution” when used in reference to an amino acid sequence refers to an amino acid of the amino acid sequence being replaced by a different amino acid (e.g., replaced by a natural or unnatural amino acid). An amino acid sequence provided herein may comprise or include one or more amino acid substitutions. Specific amino acid substitutions are denoted by commonly used colloquial nomenclature in the art of peptide sequencing to denote amino acid sequence variations. For example, when referring to SEQ ID NO: 7 (below), an “amino acid substitution at G21” refers to the glycine (G) at position 21 of the amino acid sequence being replaced by a different amino acid (e.g., a natural or unnatural amino acid other than glycine). Also for example, when referring to SEQ ID NO: 7, the amino acid substitution “G21K” refers to replacing the glycine (G) at position 21 of the amino acid sequence of SEQ ID NO: 7 with lysine (K), resulting in an amino acid sequence represented by SEQ ID NO: 71 (below).

| 1 | 2 | 3 | 4 | 5 | 6 | 7 | 8 | 9 | 10 | 11 | 12 | 13 | 14 | 15 | 16 | 17 | 18 | 19 | 20 | 21 | (Position #) |
|---|---|---|---|---|---|---|---|---|---|---|---|---|---|---|---|---|---|---|---|---|---|
| G | I | F | S | K | L | X¹ | G | K | K | X² | K | N | L | L | I | S | G | L | K | G | (SEQ ID NO: 7) |
| G | I | F | S | K | L | X¹ | G | K | K | X² | K | N | L | L | I | S | G | L | K | K | (SEQ ID NO: 71) |

The term “amino acid addition” when used in reference to an amino acid sequence refers to an amino acid (e.g., a natural or unnatural amino acid) being inserted between two amino acids of the amino acid sequence, or added at either end of the sequence. Standard colloquial nomenclature is used to represent specific amino additions (e.g., when referring to SEQ ID NO: 7, “K20_G21insX” denotes that a hypothetical amino acid X is inserted between amino acids K20 and G21 of the amino acid sequence). In certain embodiments, an amino acid sequence described herein can comprise 0, 1, 2, 3, 4, 5, 6, 7, 8, 9, or 10 amino acid additions.

The term “amino acid deletion” when used in reference to an amino acid sequence refers to an amino acid of the amino acid sequence being deleted from the amino acid sequence. Standard colloquial nomenclature is used to represent specific amino deletions (e.g., when referring to SEQ ID NO: 7, “G21del” denotes that the amino acid G21 is deleted from the sequence). In certain embodiments, an amino acid sequence described herein can comprise 0, 1, 2, 3, 4, 5, 6, 7, 8, 9, or 10 amino acid deletions.

The terms “stapled” and “crosslinked” are used interchangeably and refer to peptides wherein two amino acids (i.e., crosslinked amino acids) are connected via a crosslink (i.e., staple) to form a macrocycle. The terms “crosslink” and “staple” are used interchangeably and refer to a covalent linking moiety other than the peptide backbone which connects a pair of crosslinked amino acids to form a macrocycle. In certain embodiments, the crosslink is attached to the α-positions of the crosslinked amino acids. In certain embodiments, the crosslinked amino acids are separated by 3 amino acids in the amino acid sequence, forming an “i+4 crosslink.” In certain embodiments, the crosslinked amino acids are separated by 4 amino acids in the amino acid sequence, forming an “i+5 crosslink.” In certain embodiments, the crosslinked amino acids are separated by 6 amino acids in the amino acid sequence, forming an “i+7 crosslink.” In certain embodiments, the crosslinked amino acids are separated by 7 amino acids in the amino acid sequence, forming an “i+8 crosslink.”

The terms “dithio crosslink” and “dithio staple” are used interchangeably and refer to a crosslink comprising two thioethers (i.e., two —S— groups). A “dithio-stapled” or “dithio-crosslinked” peptide is a stapled peptide comprising one or more internal dithio crosslinks.

For example, the follow structure shows a dithio-stapled (i.e., dithio-crosslinked) peptide of the amino acid sequence GX¹GGGX² (SEQ ID NO: 72), wherein X¹ and X² are amino acids connected via an i+4 dithio crosslink of the formula (mxy), and the peptide comprises a C-terminus amidated with —NH₂:

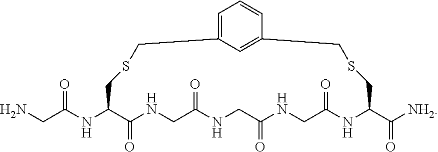 [SMILES: NCC(=O)N[C@H]1CSCc2cccc(c2)CSC[C@@H](C(N)=O)NC(=O)CNC(=O)CNC(=O)CNC1=O]

In certain embodiments, the stapled peptides provided herein are based on the amino acid sequence of the antimicrobial peptide Esculentin-1A. Esculentins are a class of cytotoxic peptides with antibacterial and antifungal activity that were originally found in the skin secretions of many species of frogs and toads. Esculentin-1A is a particular antimicrobial peptide (AMP) originally isolated from frog skin. The amino acid sequence of the peptide known as “Esculentin-1A” is provided below.

Esculentin-1A:
(SEQ ID NO: 73)
GIFSKLAGKKIKNLLISGLKNVG
KEVGMDVVRTGIDIAGCKIKGEC

As used herein, “stapled antimicrobial peptide” (i.e., “StAMP”) refers to a stapled peptide having antimicrobial (e.g., antibacterial) activity.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the peptides of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the peptides of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Throughout the present disclosure, references to “the peptide” and “a peptide” provided herein are intended to encompass peptides comprising any amino acid sequence provided herein (including any disclosed amino acid substitutions, additions, deletions, and/or modifications), and pharmaceutically acceptable salts, stereoisomers, tautomers, isotopically labeled derivatives, solvates, hydrates, polymorphs, co-crystals, and prodrugs thereof as described herein.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a peptide described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a peptide described herein refers to an amount sufficient to elicit the desired biological response. An effective amount of a peptide described herein may vary depending on such factors as the desired biological endpoint, severity of side effects, disease, or disorder, the identity, pharmacokinetics, and pharmacodynamics of the particular peptide, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a peptide described herein in a single dose. In certain embodiments, an effective amount is the combined amounts of a peptide described herein in multiple doses.

A “therapeutically effective amount” of a peptide described herein is an amount sufficient to provide a therapeutic benefit in the treatment of a condition or to delay or minimize one or more symptoms associated with the condition. A therapeutically effective amount of a peptide means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

A “prophylactically effective amount” of a peptide described herein is an amount sufficient to prevent a condition, or one or more symptoms associated with the condition or prevent its recurrence. A prophylactically effective amount of a peptide means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “infectious disease” refers to any disease caused by a pathogen (i.e., pathogenic microorganisms). An infectious disease may be caused by bacteria, viruses, parasites, or fungi. An infectious disease can be a microbial infection. A “microbial infection” refers to an infection with a microorganism, such as a fungus, bacteria or virus. Various bacterial infections include, but are not limited to, skin infections (e.g., bacterial cellulitis, wound infections), gastrointestinal infections, throat infections (e.g., strep throat), urinary tract infections (UTIs), genito-urinary infections, sexually-transmitted diseases (e.g., gonorrhea, chlamydia, syphilis), pulmonary infections (e.g., pneumonia, pneumococcal pneumonia, tuberculosis, whooping cough), food poisoning, sepsis, bacterial meningitis, Lyme disease, cholera, botulism, tetanus, anthrax, blood infections, and systemic infections. In certain embodiments, the microbial infection is an infection with bacteria, i.e., a “bacterial infection.” In certain embodiments, the bacteria are Gram-negative bacteria. In certain embodiments, the bacteria are Gram-positive bacteria.

“Gram-negative bacteria” were first defined by their ability not to retain Gram staining; however, since then Gram-negative bacteria have been further defined as bacteria generally having a cell wall with a thin peptidoglycan layer and have an outer lipid membrane.

In certain embodiments, the Gram-negative bacteria is an Escherichia species. In certain embodiments, the Gram-negative bacteria is an Escherichia coli (E. coli) strain. In certain embodiments, the Gram-negative bacteria is an Escherichia albertii, Escherichia blattae, Escherichia fergusonii, Escherichia hermannii, or Escherichia vulneris strain.

In certain embodiments, the Gram-negative bacteria is a Pseudomonas species. In certain embodiments, the Gram-negative bacteria is a Pseudomonas aeruginosa strain. In certain embodiments, the Gram-negative bacteria is a Pseudomonas alcaligenes, Pseudomonas anguilliseptica, Pseudomonas argentinensis, Pseudomonas borbori, Pseudomonas citronellolis, Pseudomonas flavescens, Pseudomonas mendocina, Pseudomonas nitroreducens, Pseudomonas oleovorans, Pseudomonas pseudoalcaligenes, Pseudomonas resinovorans, Pseudomonas straminea, Pseudomonas chlororaphis, Pseudomonas fluorescens, Pseudomonas pertucinogena, Pseudomonas putida, Pseudomonas stutzeri, or Pseudomonas syringae strain.

In certain embodiments, the Gram-negative bacteria is a Klebsiella species. In certain embodiments, the Gram-negative bacteria is a Klebsiella granulomatis, Klebsiella oxytoca, Klebsiella pneumoniae, Klebsiella terrigena, or Klebsiella planticola strain.

In certain embodiments, the Gram-negative bacteria is a Salmonella species. In certain embodiments, the Gram-negative bacteria is a Salmonella bongori strain or Salmonella enterica strain, e.g., Salmonella typhi.

In certain embodiments, the Gram-negative bacteria is an Acinetobacter species. In certain embodiments, the Gram-negative bacteria is an Acinetobacter baumannii strain. In certain embodiments, the Gram-negative bacteria is an Acinetobacter baylyi, Acinetobacter bouvetii, Acinetobacter calcoaceticus, Acinetobacter gerneri, Acinetobacter grimontii, Acinetobacter haemolyticus, Acinetobacter johnsonii, Acinetobacter junii, Acinetobacter lwoffii, Acinetobacter parvus, Acinetobacter pittii, Acinetobacter radioresistens, Acinetobacter schindleri, Acinetobacter tandoii, Acinetobacter tjernbergiae, Acinetobacter towneri, Acinetobacter ursingii, or Acinetobacter gyllenbergii strain.

In certain embodiments, the Gram-negative bacteria is a Stenotrophomonas species. In certain embodiments, the Gram-negative bacteria is a Stenotrophomonas acidaminiphila, Stenotrophomonas bentonitica, Stenotrophomonas chelatiphaga, Stenotrophomonas daejeonensis, Stenotrophomonas dokdonensis, Stenotrophomonas ginsengisoli, Stenotrophomonas humi, Stenotrophomonas indicatrix, Stenotrophomonas koreensis, Stenotrophomonas lactitubi, Stenotrophomonas maltophilia, Stenotrophomonas nitritireducens, Stenotrophomonas pavanii, Stenotrophomonas pictorum, Stenotrophomonas rhizophila, Stenotrophomonas terrae, or a Stenotrophomonas tumulicola strain.

In certain embodiments, the Gram-negative bacteria is a Burkholderia species. In certain embodiments, the bacteria is a Burkholderia cepacia, Burkholderia multivorans, Burkholderia cenocepacia, Burkholderia vietnamiensis, Burkholderia stabilis, Burkholderia ambifaria, Burkholderia dolosa, Burkholderia anthina, Burkholderia pyrrocinia, or Burkholderia ubonensis strain.

In certain embodiments, the Gram-negative bacteria is a Neisseria species. In certain embodiments, the bacteria is a Neisseria meningitidis or Neisseria gonorrhoeae strain.

In certain embodiments, the Gram-negative bacteria is an Enterobacter species. In certain embodiments, the bacteria is an Enterobacter amnigenus, Enterobacter arachidis, Enterobacter asburiae, Enterobacter cancerogenous, Enterobacter cloacae, Enterobacter cowanii, Enterobacter dissolvens, Enterobacter gergoviae, Enterobacter helveticus, Enterobacter hormaechei, Enterobacter intermedius, Enterobacter kobei, Enterobacter ludwigii, Enterobacter mori, Enterobacter nimipressuralis, Enterobacter oryzae, Enterobacter pulveris, Enterobacter pyrinus, Enterobacter radicincitans, Enterobacter taylorae, Enterobacter turicensis, or Enterobacter soli strain.

In certain embodiments, the Gram-negative bacteria is a Pasteurella species. In certain embodiments, the bacteria is a Pasteurella aerogenes, Pasteurella anatis, Pasteurella avium, Pasteurella bettyae, Pasteurella caballi, Pasteurella canis, Pasteurella dagmatis, Pasteurella gallicida, Pasteurella gallinarum, Pasteurella granulomatis, Pasteurella langaaensis, Pasteurella lymphangitidis, Pasteurella mairii, Pasteurella multocida, Pasteurella oralis, Pasteurella pneumotropica, Pasteurella skyensis, Pasteurella stomatis, Pasteurella testudinis, Pasteurella trehalosi, Pasteurella ureae, or Pasteurella volantium strain.

In certain embodiments, the Gram-negative bacteria is a Proteus species. In certain embodiments, the bacteria is a Proteus hauseri, Proteus mirabilis, Proteus myxofaciens, Proteus penneri, or Proteus vulgaris strain.

In certain embodiments, the Gram-negative bacteria is a Citrobacter species. In certain embodiments, the bacteria is a Citrobacter amalonaticus, Citrobacter braakii, Citrobacter diversus, Citrobacter europaeus, Citrobacter farmer, Citrobacter freundii, Citrobacter gillenii, Citrobacter koseri, Citrobacter murliniae, Citrobacter pasteurii, Citrobacter rodentium, Citrobacter sedlakii, Citrobacter werkmanii, Citrobacter youngae, or Citrobacter portucalensis strain.

In certain embodiments, the Gram-negative bacteria is a Helicobacter species. In certain embodiments, the bacteria is a Helicobacter acinonychis, Helicobacter ailurogastricus, Helicobacter anseris, Helicobacter aurati, Helicobacter apri, Helicobacter baculiformis, Helicobacter bilis, Helicobacter bizzozeronii, Helicobacter brantae, Helicobacter canadensis, Helicobacter canicola, Helicobacter canis, Helicobacter cetorum, Helicobacter cholecystus, Helicobacter cinaedi, Helicobacter cynogastricus, Helicobacter equorum, Helicobacter felis, Helicobacter fennelliae, Helicobacter ganmani, Helicobacter heilmannii, Helicobacter hepaticus, Helicobacter himalayensis, Helicobacter japonicus, Helicobacter mesocricetorum, Helicobacter macacae, Helicobacter marmotae, Helicobacter mastomyrinus, Helicobacter mesocricetorum, Helicobacter muridarum, Helicobacter mustelae, Helicobacter pametensis, Helicobacter pullorum, Helicobacter pylori, Helicobacter rappini, Helicobacter rodentium, Helicobacter saguini, Helicobacter salomonis, Helicobacter suis, Helicobacter trogontum, Helicobacter typhlonius, Helicobacter valdiviensis, or Helicobacter winghamensis strain.

In certain embodiments, the Gram-negative bacteria is a Campylobacter species. In certain embodiments, the bacteria is a Campylobacter avium, Campylobacter butzleri, Campylobacter canadensis, Campylobacter cinaedi, Campylobacter coli, Campylobacter concisus, Campylobacter corcagiensis, Campylobacter cryaerophilus, Campylobacter cuniculorum, Campylobacter curvus, Campylobacter fennelliae, Campylobacter fetus, Campylobacter gracilis, Campylobacter helveticus, Campylobacter hepaticus, Campylobacter hominis, Campylobacter hyoilei, Campylobacter hyointestinalis, Campylobacter insulaenigrae, Campylobacter jejuni, Campylobacter lanienae, Campylobacter lari, Campylobacter mucosalis, Campylobacter mustelae, Campylobacter nitrofigilis, Campylobacter peloridis, Campylobacter pylori, Campylobacter rectus, Campylobacter showae, Campylobacter sputorum, Campylobacter subantarcticus, Campylobacter upsaliensis, Campylobacter ureolyticus, or Campylobacter volucris strain.

“Gram-positive bacteria” are bacteria that take up the crystal violet color in the Gram staining test, and generally have cell walls comprising a thick peptidoglycan layer and no outer lipid membrane.

In certain embodiments, the Gram-positive bacteria is a Staphylococcus species. In certain embodiments, the bacteria is a Staphylococcus aureus (S. aureus). In certain embodiments, the bacteria is methicillin-resistant Staphylococcus aureus (MRSA). In certain embodiments, the bacteria is a Staphylococcus epidermidis (S. epidermidis) strain. In certain embodiments, the bacteria is a Staphylococcus auricularis, Staphylococcus carnosus, Staphylococcus condimenti, Staphylococcus massiliensis, Staphylococcus piscifermentans, Staphylococcus simulans, Staphylococcus capitis, Staphylococcus caprae, Staphylococcus saccharolyticus, Staphylococcus devriesei, Staphylococcus haemolyticus, Staphylococcus hominis, Staphylococcus chromogenes, Staphylococcus felis, Staphylococcus delphini, Staphylococcus hyicus, Staphylococcus intermedius, Staphylococcus lutrae, Staphylococcus microti, Staphylococcus muscae, Staphylococcus pseudintermedius, Staphylococcus rostri, Staphylococcus schleiferi, Staphylococcus lugdunensis, Staphylococcus arlettae, Staphylococcus cohnii, Staphylococcus equorum, Staphylococcus gallinarum, Staphylococcus kloosii, Staphylococcus leei, Staphylococcus nepalensis, Staphylococcus saprophyticus, Staphylococcus succinus, Staphylococcus xylosus, Staphylococcus fleurettii, Staphylococcus lentus, Staphylococcus sciuri, Staphylococcus stepanovicii, Staphylococcus vitulinus, Staphylococcus simulans, Staphylococcus pasteuri, or Staphylococcus warneri strain.

In certain embodiments, the Gram-positive bacteria is a Streptococcus species. In certain embodiments, the bacteria is a Streptococcus agalactiae, Streptococcus anginosus, Streptococcus bovis, Streptococcus canis, Streptococcus constellatus, Streptococcus dysgalactiae, Streptococcus equinus, Streptococcus iniae, Streptococcus intermedius, Streptococcus mitis, Streptococcus mutans, Streptococcus oralis, Streptococcus parasanguinis, Streptococcus peroris, Streptococcus pneumoniae, Streptococcus pseudopneumoniae, Streptococcus pyogenes, Streptococcus ratti, Streptococcus salivarius, Streptococcus tigurinus, Streptococcus thermophilus, Streptococcus sanguinis, Streptococcus sobrinus, Streptococcus suis, Streptococcus uberis, Streptococcus vestibularis, Streptococcus viridans, or Streptococcus zooepidemicus strain.

In certain embodiments, the Gram-positive bacteria is an Enterococcus species. In certain embodiments, the bacteria is an Enterococcus avium, Enterococcus durans, Enterococcus faecalis, Enterococcus faecium, Enterococcus gallinarum, Enterococcus hirae, or Enterococcus solitarius strain.

In certain embodiments, the Gram-positive bacteria is a Listeria species. In certain embodiments, the bacteria is a Listeria fleischmannii, Listeria grayi, Listeria innocua, Listeria ivanovii, Listeria marthii, Listeria monocytogenes, Listeria rocourtiae, Listeria seeligeri, Listeria weihenstephanensis, or Listeria welshimeri strain.

In certain embodiments, the Gram-positive bacteria is a Clostridium species. In certain embodiments, the bacteria is a Clostridium acetobutylicum, Clostridium argentinense, Clostridium aerotolerans, Clostridium baratii, Clostridium beijerinckii, Clostridium bifermentans, Clostridium botulinum, Clostridium butyricum, Clostridium cadaveris, Clostridium cellulolyticum, Clostridium chauvoei, Clostridium clostridioforme, Clostridium colicanis, Clostridium difficile, Clostridium estertheticum, Clostridium fallax, Clostridium feseri, Clostridium formicaceticum, Clostridium histolyticum, Clostridium innocuum, Clostridium kluyveri, Clostridium ljungdahlii, Clostridium lavalense, Clostridium leptum, Clostridium novyi, Clostridium oedematiens, Clostridium paraputrificum, Clostridium perfringens (Alias, Clostridium welchii), Clostridium phytofermentans, Clostridium piliforme, Clostridium ragsdalei, Clostridium ramosum, Clostridium scatologenes, Clostridium septicum, Clostridium sordellii, Clostridium sporogenes, Clostridium sticklandii, Clostridium tertium, Clostridium tetani, Clostridium thermocellum, Clostridium thermosaccharolyticum, or Clostridium tyrobutyricum strain.

As used herein, an “antibiotic-resistant bacterial infection” is a bacterial infection caused by antibiotic-resistant bacteria. “Antibiotic resistance” occurs when bacteria evolve mechanisms that protect them from the effects of antibiotics. Microbes resistant to multiple antimicrobials are referred to as “multidrug resistant” (MDR). In certain embodiments, methods herein are for treating multidrug resistant bacterial infections. In certain embodiments, methods herein are for killing and/or inhibiting the growth of multidrug resistant bacteria.

As used herein the term “inhibit” or “inhibition” in the context of bacterial growth, for example, refers to a reduction in the rate of growth of the bacteria. In some embodiments, the term refers to a reduction in the rate of bacterial growth to a level that is statistically significantly lower than an initial rate (e.g., the rate of bacterial growth before administration or application of an antimicrobial peptide provided herein). In some embodiments, the term refers to a reduction in the rate of bacterial growth to a rate that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial rate (e.g., the rate of bacterial growth before administration or application of an antimicrobial peptide provided herein).

A peptide described herein “selectively” kills and/or inhibits the growth of one type of bacteria over another (e.g., selectively kills and/or inhibits the growth of Gram-negative bacteria over Gram-positive bacteria) if it kills and/or inhibits the growth of one type of bacteria to a greater extent than the other. A peptide described herein “selectively” kills and/or inhibits the growth of microbial cells over mammalian cells (e.g., selectively kills and/or inhibits the growth of bacterial cells over mammalian cells) if it kills and/or inhibits the growth of the microbial cells to a greater extent than the mammalian cells. A peptide described herein “selectively” lyses microbial cells over mammalian cells (e.g., selectively lyses bacterial cells over mammalian cells) if it lyses the microbial cells to a greater extent than the mammalian cells. A peptide described herein is “selectively” cytotoxic to microbial cells (e.g., bacterial cells) over mammalian cells if it is toxic (e.g., by lysing, killing, or otherwise damaging) to microbial cells (e.g., bacterial cells) to a greater extent than the mammalian cells. In certain embodiments, the selectivity in any of the foregoing embodiments is at least 1.1-fold, at least 1.5-fold, 2-fold, at least 3-fold, at least 5-fold, at least 10-fold, at least 30-fold, at least 50-fold, at least 100-fold, at least 300-fold, at least 500-fold, at least 1,000-fold, at least 3,000-fold, at least 5,000-fold, at least 10,000-fold, at least 30,000-fold, at least 50,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Peptides described herein can comprise one or more asymmetric centers, and thus can exist in various stereoisomeric forms, e.g., enantiomers and/or diastereomers. For example, the peptides described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses peptides as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond. Additionally, the bond [symbol] or [symbol] is a double or triple bond.

Unless otherwise provided, formulae and structures depicted herein include peptides that do not include isotopically enriched atoms, and also include peptides that include isotopically enriched atoms (“isotopically labeled derivatives”). For example, peptides having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such peptides are useful, for example, as analytical tools or probes in biological assays. The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like.

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). The one or more carbon-carbon double bonds can be internal (such as in 2-butenyl) or terminal (such as in 1-butenyl). In an alkenyl group, a C═C double bond for which the stereochemistry is not specified (e.g., —CH═CHCH₃ or

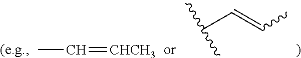

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”).

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). The one or more carbon-carbon triple bonds can be internal (such as in 2-butynyl) or terminal (such as in 1-butynyl).

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”).

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 6 ring carbon atoms (“C₃₋₆ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

The term “aryl” refers to a radical of a monocyclic or polycyclic (e.g., bicyclic or tricyclic) 4n+2 aromatic ring system (e.g., having 6, 10, or 14 n electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom or the ring that does not contain a heteroatom.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A chemical moiety is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, heteroaryl, acyl groups are optionally substituted. In general, the term “substituted” when referring to a chemical group means that at least one hydrogen present on the group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The invention is not limited in any manner by the exemplary substituents described herein.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SCN, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring; and each X⁻ is a counterion.

In certain embodiments, each substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —N₃, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thio” refers to a sulfur-containing group. The term “thiol” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S—SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa}, R^{bb}, and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group. The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen. The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen. The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{aa}, —C(═O)OR^{a}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

A “counterion” is a charged group associated with an oppositely charged group in order to maintain electronic neutrality. An “anionic counterion” is a negatively charged group associated with a positively charged group. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

These and other exemplary substituents are described in more detail in the Detailed Description, Examples, and Claims. The invention is not limited in any manner by the above exemplary listing of substituents.

### DETAILED DESCRIPTION OF CERTAIN EMBODIMENTS

Provided herein are stapled antimicrobial peptides (StAMPs), pharmaceutical compositions thereof, and kits comprising the same. The peptides provided herein have antimicrobial properties and can therefore be used to treat and/or prevent infectious diseases (e.g., bacterial infections) in a subject, to kill bacteria, and/or inhibit the growth of bacteria, etc. Also provided herein are unstapled peptides which can serve as synthetic precursors to the stapled peptides provided herein. In one aspect, provided herein are StAMPs based on the amino acid sequence of Esculentin-1A that comprise one or more dithio staples.

### Stapled Peptides

In one aspect, provided herein are stapled (i.e., crosslinked) peptides. The stapled peptides provided herein are based on the amino acid sequence of the antimicrobial peptide Esculentin-1A and include one or more staples (e.g., dithio staples). In certain embodiments, the placement of the one or more staples within the peptide may confer advantageous properties (e.g., improved antimicrobial activity, selectivity for killing Gram-negative bacteria, and/or reduced toxicity) and/or allow for certain stapling technology to be used (e.g., thiol-stapling).

In certain embodiments, the stapled peptides provided herein comprise one staple (i.e., crosslink) that connect the amino acid residues X¹ and X², as included in the amino acid sequences provided herein. In certain embodiments, the stapled peptides provided herein comprise two staples (i.e., crosslinks) that connect the amino acid residues X¹ and X², and X³ and X⁴, as included in the amino acid sequences provided herein.

In certain embodiments, the stapled peptides provided herein comprise one dithio staple (i.e., dithio crosslink) that connect the amino acid residues X¹ and X², as included in the amino acid sequences provided herein. In certain embodiments, the stapled peptides provided herein comprise two dithio staples (i.e., dithio crosslinks) that connect the amino acid residues X¹ and X², and X³ and X⁴, as included in the amino acid sequences provided herein.

### Amino Acid Sequences

One aspect of the present disclosure relates to StAMPs based on the sequence of Esculentin-1A comprising exactly one internal crosslink (i.e., singly stapled peptides).

For instance, provided herein are peptides, and pharmaceutically acceptable salts thereof, comprising 10 or more contiguous amino acids of Esculentin-1A (SEQ ID NO: 73) and comprising exactly one internal crosslink connecting two crosslinked amino acids, wherein:
two amino acids of the 10 or more contiguous amino acids of Esculentin-1A are independently substituted by X¹ and X²; and
X¹ and X² are independently the crosslinked amino acids;
optionally wherein the amino acid sequence includes 1 to 3 amino acid substitutions, inclusive, at positions other than X¹ and X².

In certain embodiments, the peptide is based on 10 or more, 11 or more, 12 or more, 13 or more, 14 or more, 15 or more, 16 or more, 17 or more, 18 or more, 19 or more, or 20 or more contiguous amino acids of Esculentin-1A (SEQ ID NO: 73). In certain embodiments, the peptide is based on 15 or more contiguous amino acids of Esculentin-1A (SEQ ID NO: 73). In certain embodiments, the peptide is based on 20 or more contiguous amino acids of Esculentin-1A (SEQ ID NO: 73). In certain embodiments, the peptide is based on 10-30 contiguous amino acids, inclusive, of Esculentin-1A (SEQ ID NO: 73). In certain embodiments, the peptide is based on 15-25 contiguous amino acids, inclusive, of Esculentin-1A (SEQ ID NO: 73). In certain embodiments, the peptide is based on 18-23 contiguous amino acids, inclusive, of Esculentin-1A (SEQ ID NO: 73). In certain embodiments, the peptide is based on 21 contiguous amino acids of Esculentin-1A (SEQ ID NO: 73). In certain embodiments, the 21 contiguous amino acids of Esculentin-1A are the following: GIFSKLAGKKIKNLLISGLKN (SEQ ID NO: 75). In certain embodiments, the peptide is based on the following amino acid sequence: GIFSKLAGKKIKNLLISGLKG (SEQ ID NO: 76), which corresponds to SEQ ID NO: 75 with a N21G amino acid substitution.

In certain embodiments, the amino acid sequence includes one or more (e.g., 1, 2, 3, 4, 5, 6, 7, 8, 9, 10) amino acid substitutions at positions other than X¹ and X². The amino acids can be independently substituted by any natural or unnatural amino acid, including, but not limited to, those amino acids provided herein. In certain embodiments, the amino acid sequence comprises 1 amino acid substitution. In certain embodiments, the amino acid sequence comprises 1 or 2 amino acid substitutions. In certain embodiments, the amino acid sequence comprises 1 to 3 amino acid substitutions, inclusive. In certain embodiments, the amino acid sequence comprises 1 to 4 amino acid substitutions, inclusive. In certain embodiments, the amino acid sequence comprises 1 to 5 amino acid substitutions, inclusive. In certain embodiments, the amino acid sequence comprises 1 to 6 amino acid substitutions, inclusive. In certain embodiments, the amino acid sequence comprises 1 to 7 amino acid substitutions, inclusive. In certain embodiments, the amino acid sequence comprises 1 to 8 amino acid substitutions, inclusive. In certain embodiments, the amino acid sequence comprises 1 to 9 amino acid substitutions, inclusive. In certain embodiments, the amino acid sequence comprises 1 to 10 amino acid substitutions, inclusive.

In certain embodiments, the peptide comprises 20 or more contiguous amino acids of Esculentin-1A (SEQ ID NO: 73); optionally wherein the amino acid sequence includes 1 to 9 amino acid substitutions, inclusive, at positions other than X¹ and X². In certain embodiments, the peptide comprises 20 or more contiguous amino acids of Esculentin-1A (SEQ ID NO: 73); optionally wherein the amino acid sequence includes 1 to 6 amino acid substitutions, inclusive, at positions other than X¹ and X². In certain embodiments, the peptide comprises 20 or more contiguous amino acids of Esculentin-1A (SEQ ID NO: 73); optionally wherein the amino acid sequence includes 1 to 3 amino acid substitutions, inclusive, at positions other than X¹ and X².

In certain embodiments, the peptide comprises SEQ ID NO: 75, optionally wherein the amino acid sequence includes 1 to 9 amino acid substitutions, inclusive, at positions other than X¹ and X². In certain embodiments, the peptide comprises SEQ ID NO: 75, optionally wherein the amino acid sequence includes 1 to 6 amino acid substitutions, inclusive, at positions other than X¹ and X². In certain embodiments, the peptide comprises SEQ ID NO: 75, optionally wherein the amino acid sequence includes 1 to 3 amino acid substitutions, inclusive, at positions other than X¹ and X².

In certain embodiments, the peptide comprises SEQ ID NO: 76, optionally wherein the amino acid sequence includes 1 to 9 amino acid substitutions, inclusive, at positions other than X¹ and X². In certain embodiments, the peptide comprises SEQ ID NO: 76, optionally wherein the amino acid sequence includes 1 to 6 amino acid substitutions, inclusive, at positions other than X¹ and X². In certain embodiments, the peptide comprises SEQ ID NO: 76, optionally wherein the amino acid sequence includes 1 to 3 amino acid substitutions, inclusive, at positions other than X¹ and X².

In certain embodiments of any of the foregoing embodiments, X¹ and X² are separated by 3 amino acids, forming an “i+4 crosslink.” In certain embodiments, X¹ and X² are separated by 4 amino acids, forming an “i+5 crosslink.” In certain embodiments, X¹ and X² are separated by 5 amino acids, forming an “i+6 crosslink.” In certain embodiments, X¹ and X² are separated by 6 amino acids, forming an “i+7 crosslink.”

In certain embodiments, a stapled (i.e., crosslinked) peptide provided herein comprises the following amino acid sequence:

(SEQ ID NO: 7)
G I F S K L X¹ G K K X² K N L L I S G L K G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X². In certain embodiments, X¹ and X² are connected via a dithio crosslink (e.g., (mxy), (pxy), (but), (bbn), (bbf), (bbp), (pfb), (hfb)).

In certain embodiments, the stapled (i.e., crosslinked) peptide comprising SEQ ID NO: 7 comprises the following amino acid sequence:

(SEQ ID NO: 74)
G I F S K L X¹ G K K X² K N L L I S G L K G G G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X². In certain embodiments, X¹ and X² are connected via a dithio crosslink (e.g., (mxy), (pxy), (but), (bbn), (bbf), (bbp), (pfb), (hfb)).

In certain embodiments, the stapled (i.e., crosslinked) peptide comprises one of the following amino acid sequence:

(SEQ ID NO: 65)
G I F S K L X¹ G K K X² K N L L I S G L K G G G G,
(SEQ ID NO: 66)
G I F S K L X¹ G K K X² K N L L I S G L K G G G K,
(SEQ ID NO: 67)
G I F S K L X¹ G K K X² K N L L I S G L K G A G,
(SEQ ID NO: 68)
G I F S K L X¹ G K K X² K N L L I S G L K G A K,
(SEQ ID NO: 69)
G I F S K L X¹ A K K X² K N L L I S G L K G,
(SEQ ID NO: 70)
G I F S K L X¹ G K K X² K N L L I S A L K G,
(SEQ ID NO: 77)
G I F S K L X¹ A K K X² K N L L I S A L K G,

or a pharmaceutically acceptable salt thereof, wherein X¹ and X² are independently amino acids connected via a crosslink. In certain embodiments, X¹ and X² are connected via a dithio crosslink (e.g., (mxy), (pxy), (but), (bbn), (bbf), (bbp), (pfb), (hfb)).

In other embodiments, a stapled (i.e., crosslinked) peptide may comprise one of the following amino acid sequences:

(SEQ ID NO: 1)
X¹ I F S X² L A G K K I K N L L I S G L K G,
(SEQ ID NO: 2)
G X¹ F S K X² A G K K I K N L L I S G L K G,
(SEQ ID NO: 3)
G I X¹ S K L X² G K K I K N L L I S G L K G,
(SEQ ID NO: 4)
G I F X¹ K L A X² K K I K N L L I S G L KG,
(SEQ ID NO: 5)
G I F S X¹ L A G X² K I K N L L I S G L K G,
(SEQ ID NO: 6)
G I F S K X¹ A G K X² I K N L L I S G L K G,
(SEQ ID NO: 8)
G I F S K L A X¹ K K I X² N L L I S G L K G,
(SEQ ID NO: 9)
G I F S K L A G X¹ K I K X² L L I S G L K G,
(SEQ ID NO: 10)
G I F S K L A G K X¹ I K N X² L I S G L K G,
(SEQ ID NO: 11)
G I F S K L A G K K X¹ K N L X² I S G L K G,
(SEQ ID NO: 12)
G I F S K L A G K K I X¹ N L L X² S G L K G,
(SEQ ID NO: 13)
G I F S K L A G K K I K X¹ L L I X² G L K G,
(SEQ ID NO: 14)
G I F S K L A G K K I K N X¹ L I S X² L K G,
(SEQ ID NO: 15)
G I F S K L A G K K I K N L X¹ I S G X² K G,
(SEQ ID NO: 16)
G I F S K L A G K K I K N L L X¹ S G L X² G,
(SEQ ID NO: 17)
G I F S K L A G K K I K N L L I X¹ G L K X²,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X². In certain embodiments, X¹ and X² are connected via a dithio crosslink (e.g., (mxy), (pxy), (but), (bbn), (bbf), (bbp), (pfb), (hfb)).

In other embodiments, a stapled (i.e., crosslinked) peptide may comprise one of the following amino acid sequences:

(SEQ ID NO: 18)
X¹ I F S K L A X² K K I K N L L I S G L K G,
(SEQ ID NO: 19)
G X¹ F S K L A G X² K I K N L L I S G L K G,
(SEQ ID NO: 20)
G I X¹ S K L A G K X² I K N L L I S G L K G,
(SEQ ID NO: 21)
G I F X¹ K L A G K K X² K N L L I S G L K G,
(SEQ ID NO: 22)
G I F S X¹ L A G K K I X² N L L I S G L K G,
(SEQ ID NO: 23)
G I F S K X¹ A G K K I K X² L L I S G L K G,
(SEQ ID NO: 24)
G I F S K L A X¹ K K I K N L X² I S G L K G,
(SEQ ID NO: 25)
G I F S K L A G X¹ K I K N L L X² S G L K G,
(SEQ ID NO: 26)
G I F S K L A G K X¹ I K N L L I X² G L K G,
(SEQ ID NO: 27)
G I F S K L A G K K X¹ K N L L I S X² L K G,
(SEQ ID NO: 28)
G I F S K L A G K K I X¹ N L L I S G X² K G,
(SEQ ID NO: 29)
G I F S K L A G K K I K X¹ L L I S G L X² G,
(SEQ ID NO: 30)
G I F S K L A G K K I K N X¹ L I S G L K X²,
(SEQ ID NO: 31)
G I F S K L X¹ G K K I K N X² L I S G L K G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X². In certain embodiments, X¹ and X² are connected via a dithio crosslink (e.g., (mxy), (pxy), (but), (bbn), (bbf), (bbp), (pfb), (hfb)).

Also provided herein are stapled (i.e., crosslinked) peptides comprising the following amino acid sequence:

(SEQ ID NO: 32)
G I F S K L X¹ G K K X² K N L L I X³ G L K X⁴,

or a pharmaceutically acceptable salt thereof, wherein:
X¹, X², X³, and X⁴ are independently amino acids;
X¹ and X² are connected via a crosslink, and X³ and X⁴ are connected via a crosslink; and optionally wherein the amino acid sequence includes 1 to 6 amino acid substitutions, inclusive, at positions other than X¹, X², X³, and X⁴. In certain embodiments, X¹ and X² are connected via a dithio crosslink (e.g., mxy, pxy, but, bbn, bbf, bbp, pfb, hfb).

In certain embodiments, an amino acid sequence provided herein (e.g., SEQ ID NOs: 1-32, 65-70, 74-78) includes one or more (e.g., 1, 2, 3, 4, 5, 6, 7, 8, 9, 10) amino acid substitutions at positions other than X¹ and X². The amino acids can be independently substituted by any natural or unnatural amino acid, including, but not limited to, those amino acids provided herein. In certain embodiments, the amino acid sequence comprises 1 amino acid substitution. In certain embodiments, the amino acid sequence comprises 1 or 2 amino acid substitutions. In certain embodiments, the amino acid sequence comprises 1 to 3 amino acid substitutions, inclusive. In certain embodiments, the amino acid sequence comprises 1 to 4 amino acid substitutions, inclusive. In certain embodiments, the amino acid sequence comprises 1 to 5 amino acid substitutions, inclusive. In certain embodiments, the amino acid sequence comprises 1 to 6 amino acid substitutions, inclusive. In certain embodiments, the amino acid sequence comprises 1 to 7 amino acid substitutions, inclusive. In certain embodiments, the amino acid sequence comprises 1 to 8 amino acid substitutions, inclusive. In certain embodiments, the amino acid sequence comprises 1 to 9 amino acid substitutions, inclusive. In certain embodiments, the amino acid sequence comprises 1 to 10 amino acid substitutions, inclusive.

In certain embodiments, an amino acid sequence provided herein (e.g., SEQ ID NOs: 1-32, 65-70, 74-78) comprises at least one amino acid substitution at a position selected from G8 and G18.

In certain embodiments, an amino acid sequence provided herein (e.g., SEQ ID NOs: 1-32, 65-70, 74-78) comprises an amino acid substitution at G8. In certain embodiments, the amino acid substation at G8 is G8A.

In certain embodiments, an amino acid sequence provided herein (e.g., SEQ ID NOs: 1-32, 65-70, 74-78) comprises an amino acid substitution at G18. In certain embodiments, the amino acid substitution at G18 is G18A.

In certain embodiments, an amino acid sequence provided herein (e.g., SEQ ID NOs: 1-32, 65-70, 74-78) comprises amino acid substitutions at G8 and G18. In certain embodiments, an amino acid sequence provided herein (e.g., SEQ ID NOs: 1-32, 65-70, 74-78) comprises G8A and G18A amino acid substitutions.

In certain embodiments, an amino acid sequence provided herein (e.g., SEQ ID NOs: 1-32, 65-70, 74-78) comprises amino acid substitutions at G1. In certain embodiments, an amino acid sequence provided herein (e.g., SEQ ID NOs: 1-32, 65-70, 74-78) comprises a G1J amino acid substitution. In certain embodiments a stapled (i.e., crosslinked) peptide may comprises the amino acid sequence: JIFSKX¹AGKX²IKNLLISGLKG (SEQ ID NO: 78).

### Dithio-Stapled Peptides

Another aspect of the present disclosure relates to StAMPs based on the amino acid sequence of Esculentin-1A that include one or more dithio staples (i.e., dithio crosslinks). Provided herein are peptides, and pharmaceutically acceptable salts thereof, comprising 10 or more contiguous amino acids of Esculentin-1A (SEQ ID NO: 73) and at least one internal dithio crosslink, wherein:
at least 2 amino acids of the 10 or more contiguous amino acids of Esculentin-1A are substituted by a pair of dithio-crosslinked amino acids;
optionally wherein the amino acid sequence includes 1 to 3 additional amino acid substitutions, inclusive.

In certain embodiments, the peptide is based on 10 or more, 11 or more, 12 or more, 13 or more, 14 or more, 15 or more, 16 or more, 17 or more, 18 or more, 19 or more, or 20 or more contiguous amino acids of Esculentin-1A (SEQ ID NO: 73). In certain embodiments, the peptide is based on 15 or more contiguous amino acids of Esculentin-1A (SEQ ID NO: 73). In certain embodiments, the peptide is based on 20 or more contiguous amino acids of Esculentin-1A (SEQ ID NO: 73). In certain embodiments, the peptide is based on 10-30 contiguous amino acids, inclusive, of Esculentin-1A (SEQ ID NO: 73). In certain embodiments, the peptide is based on 15-25 contiguous amino acids, inclusive, of Esculentin-1A (SEQ ID NO: 73). In certain embodiments, the peptide is based on 18-23 contiguous amino acids, inclusive, of Esculentin-1A (SEQ ID NO: 73). In certain embodiments, the peptide is based on 21 contiguous amino acids, inclusive, of Esculentin-1A (SEQ ID NO: 73). In certain embodiments, the 21 contiguous amino acids of Esculentin-1A are the following: GIFSKLAGKKIKNLLISGLKN (SEQ ID NO: 75). In certain embodiments, the peptide is based on the following amino acid sequence: GIFSKLAGKKIKNLLISGLKG (SEQ ID NO: 76), which corresponds to SEQ ID NO: 75 with a N21G amino acid substitution.

As described herein, the peptide may comprise one or more (e.g., 1, 2) internal dithio crosslinks. In certain embodiments, 2 amino acids of the 10 or more (e.g., 20 or more) contiguous amino acids of Esculentin-1A (SEQ ID NO: 73) are substituted by a pair of dithio-crosslinked amino acids, thereby forming one dithio crosslink. In certain embodiments, 4 amino acids of the 10 or more (e.g., 20 or more) contiguous amino acids of Esculentin-1A (SEQ ID NO: 73) are independently substituted by two pairs of dithio-crosslinked amino acids, thereby forming two dithio crosslinks.

In certain embodiments, 2 amino acids of SEQ ID NO: 75 are substituted by a pair of dithio-crosslinked amino acids, thereby forming one dithio crosslink. In certain embodiments, 4 amino acids of SEQ ID NO: 75 are independently substituted by two pairs of dithio-crosslinked amino acids, thereby forming two dithio crosslinks.

In certain embodiments, 2 amino acids of SEQ ID NO: 76 are substituted by a pair of dithio-crosslinked amino acids, thereby forming one dithio crosslink. In certain embodiments, 4 amino acids of SEQ ID NO: 76 are independently substituted by two pairs of dithio-crosslinked amino acids, thereby forming two dithio crosslinks.

In certain embodiments, the amino acid sequence includes one or more (e.g., 1, 2, 3, 4, 5, 6, 7, 8, 9, 10) additional amino acid substitutions (i.e., at positions other than the dithio-crosslinked amino acids). The amino acids can be independently substituted by any natural or unnatural amino acid, including, but not limited to, those amino acids provided herein. In certain embodiments, the amino acid sequence comprises 1 additional amino acid substitution. In certain embodiments, the amino acid sequence comprises 1 or 2 additional amino acid substitutions. In certain embodiments, the amino acid sequence comprises 1 to 3 additional amino acid substitutions, inclusive. In certain embodiments, the amino acid sequence comprises 1 to 4 additional amino acid substitutions, inclusive. In certain embodiments, the amino acid sequence comprises 1 to 5 additional amino acid substitutions, inclusive. In certain embodiments, the amino acid sequence comprises 1 to 6 additional amino acid substitutions, inclusive. In certain embodiments, the amino acid sequence comprises 1 to 7 additional amino acid substitutions, inclusive. In certain embodiments, the amino acid sequence comprises 1 to 8 additional amino acid substitutions, inclusive. In certain embodiments, the amino acid sequence comprises 1 to 9 additional amino acid substitutions, inclusive. In certain embodiments, the amino acid sequence comprises 1 to 10 additional amino acid substitutions, inclusive.

In certain embodiments, the peptide comprises 20 or more contiguous amino acids of Esculentin-1A; optionally wherein the amino acid sequence includes 1 to 9 additional amino acid substitutions, inclusive (i.e., at positions other than the dithio-crosslinked amino acids). In certain embodiments, the peptide comprises 20 or more contiguous amino acids of Esculentin-1A; optionally wherein the amino acid sequence includes 1 to 6 additional amino acid substitutions, inclusive (i.e., at positions other than the dithio-crosslinked amino acids). In certain embodiments, the peptide comprises 20 or more contiguous amino acids of Esculentin-1A; optionally wherein the amino acid sequence includes 1 to 3 additional amino acid substitutions, inclusive (i.e., at positions other than the dithio-crosslinked amino acids).

In certain embodiments, the peptide comprises SEQ ID NO: 75, optionally wherein the amino acid sequence includes 1 to 9 additional amino acid substitutions, inclusive (i.e., at positions other than the dithio-crosslinked amino acids). In certain embodiments, the peptide comprises SEQ ID NO: 75, optionally wherein the amino acid sequence includes 1 to 6 additional amino acid substitutions, inclusive (i.e., at positions other than the dithio-crosslinked amino acids). In certain embodiments, the peptide comprises SEQ ID NO: 75, optionally wherein the amino acid sequence includes 1 to 3 additional amino acid substitutions, inclusive (i.e., at positions other than the dithio-crosslinked amino acids).

In certain embodiments, the peptide comprises SEQ ID NO: 76, optionally wherein the amino acid sequence includes 1 to 9 additional amino acid substitutions, inclusive (i.e., at positions other than the dithio-crosslinked amino acids). In certain embodiments, the peptide comprises SEQ ID NO: 76, optionally wherein the amino acid sequence includes 1 to 6 additional amino acid substitutions, inclusive (i.e., at positions other than the dithio-crosslinked amino acids). In certain embodiments, the peptide comprises SEQ ID NO: 76, optionally wherein the amino acid sequence includes 1 to 3 additional amino acid substitutions, inclusive (i.e., at positions other than the dithio-crosslinked amino acids).

In certain embodiments, an amino acid sequence provided herein (e.g., SEQ ID NOs: 1-32, 65-70, 74-78) comprises at least one amino acid substitution at a position selected from G8 and G18.

In certain embodiments, an amino acid sequence provided herein (e.g., SEQ ID NOs: 1-32, 65-70, 74-78) comprises an amino acid substitution at G8. In certain embodiments, the amino acid substation at G8 is G8A.

In certain embodiments, an amino acid sequence provided herein (e.g., SEQ ID NOs: 1-32, 65-70, 74-78) comprises an amino acid substitution at G18. In certain embodiments, the amino acid substation at G18 is G18A.

In certain embodiments, an amino acid sequence provided herein (e.g., SEQ ID NOs: 1-32, 65-70, 74-78) comprises amino acid substitutions at G8 and G18. In certain embodiments, an amino acid sequence provided herein (e.g., SEQ ID NOs: 1-32, 65-70, 74-78) comprises G8A and G18A amino acid substitutions.

In certain embodiments, an amino acid sequence provided herein (e.g., SEQ ID NOs: 1-32, 65-70, 74-78) comprises amino acid substitutions at G1. In certain embodiments, an amino acid sequence provided herein (e.g., SEQ ID NOs: 1-32, 65-70, 74-78) comprises a G1J amino acid substitution. In certain embodiments a stapled (i.e., crosslinked) peptide may comprises the amino acid sequence: JIFSKXIAGKX²IKNLLISGLKG (SEQ ID NO: 78).

In certain embodiments of any of the foregoing embodiments, at least one pair of dithio-crosslinked amino acids are separated by 3 amino acids, forming an “i+4 dithio crosslink.” In certain embodiments, at least one pair of dithio-crosslinked amino acids are separated by 4 amino acids, forming an “i+5 dithio crosslink.” In certain embodiments, at least one pair of dithio-crosslinked amino acids are separated by 5 amino acids, forming an “i+6 dithio crosslink.” In certain embodiments, at least one pair of dithio-crosslinked amino acids are separated by 6 amino acids, forming an “i+7 dithio crosslink.”

In certain embodiments, a dithio stapled (i.e., dithio crosslinked) peptide provided herein comprises the following amino acid sequence:

(SEQ ID NO: 7)
G I F S K L X¹ G K K X² K N L L I S G L K G,

or a pharmaceutically acceptable salt thereof, wherein:
X¹ and X² are independently amino acids connected via a dithio crosslink;
optionally wherein the amino acid sequence includes 1 to 6 amino acid substitutions, inclusive, at positions other than X¹ and X².

In certain embodiments, the dithio stapled (i.e., dithio crosslinked) peptide comprising SEQ ID NO: 7 comprises the following amino acid sequence:

(SEQ ID NO: 74)
G I F S K L X¹ G K K X² K N L L I S G L K G G G,

or a pharmaceutically acceptable salt thereof, wherein:
X¹ and X² are independently amino acids connected via a dithio crosslink;
optionally wherein the amino acid sequence includes 1 to 6 amino acid substitutions, inclusive, at positions other than X¹ and X².

In certain embodiments, X¹ and X² in SEQ ID NO: 7 are connected via a dithio crosslink selected from (mxy), (pxy), (but), (bbn), (bbf), (bbp), (pfb), and (hfb). In certain embodiments, X¹ and X² in SEQ ID NO: 7 are connected via a dithio crosslink selected from (mxy), (pxy), and (but). In certain embodiments, X¹ and X² in SEQ ID NO: 7 are connected via (mxy). In certain embodiments, X¹ and X² in SEQ ID NO: 7 are connected via (pxy). In certain embodiments, X¹ and X² in SEQ ID NO: 7 are connected via (but).

In certain embodiments, a dithio-stapled (i.e., dithio-crosslinked) peptide or pharmaceutically acceptable salt thereof provided herein is of SEQ ID NO: 7, wherein X¹ and X² are connected via (mxy). In certain embodiments, the peptide or pharmaceutically acceptable salt thereof is of SEQ ID NO: 7; X¹ and X² are connected via (mxy); and the C-terminus of the peptide is amidated with —NH₂. In certain embodiments, the peptide or pharmaceutically acceptable salt thereof is of SEQ ID NO: 7; X¹ and X² are connected via (mxy); and the C-terminus is —CO₂H.

In certain embodiments, a dithio-stapled (i.e., dithio-crosslinked) peptide or pharmaceutically acceptable salt thereof provided herein is of SEQ ID NO: 7, wherein X¹ and X² are connected via (pxy). In certain embodiments, the peptide or pharmaceutically acceptable salt thereof is of SEQ ID NO: 7; X¹ and X² are connected via (pxy); and the C-terminus of the peptide is amidated with —NH₂.

In certain embodiments, a dithio-stapled (i.e., dithio-crosslinked) peptide or pharmaceutically acceptable salt thereof provided herein is of SEQ ID NO: 7, wherein X¹ and X² are connected via (but). In certain embodiments, the peptide or pharmaceutically acceptable salt thereof is of SEQ ID NO: 7; X¹ and X² are connected via (but); and the C-terminus of the peptide is amidated with —NH₂.

In certain embodiments, X¹ and X² in SEQ ID NO: 74 are connected via a dithio crosslink selected from (mxy), (pxy), (but), (bbn), (bbf), (bbp), (pfb), and (hfb). In certain embodiments, X¹ and X² in SEQ ID NO: 74 are connected via a dithio crosslink selected from (mxy), (pxy), and (but). In certain embodiments, X¹ and X² in SEQ ID NO: 74 are connected via (mxy). In certain embodiments, X¹ and X² in SEQ ID NO: 74 are connected via (pxy). In certain embodiments, X¹ and X² in SEQ ID NO: 74 are connected via (but).

In certain embodiments, a dithio-stapled (i.e., dithio-crosslinked) peptide or pharmaceutically acceptable salt thereof provided herein is of SEQ ID NO: 74, wherein X¹ and X² are connected via (mxy). In certain embodiments, the peptide or pharmaceutically acceptable salt thereof is of SEQ ID NO: 74; X¹ and X² are connected via (mxy); and the C-terminus is —CO₂H.

In certain embodiments, X¹ and X² in any one of SEQ ID NOs: 65-70 and 77 are connected via a dithio crosslink selected from (mxy), (pxy), (but), (bbn), (bbf), (bbp), (pfb), and (hfb). In certain embodiments, X¹ and X² in any one of SEQ ID NOs: 65-70 and 77 are connected via a dithio crosslink selected from (mxy), (pxy), and (but). In certain embodiments, X¹ and X² in any one of SEQ ID NOs: 65-70 and 77 are connected via (mxy). In certain embodiments, X¹ and X² in any one of SEQ ID NOs: 65-70 and 77 are connected via (pxy). In certain embodiments, X¹ and X² in any one of SEQ ID NOs: 65-70 and 77 are connected via (but).

In certain embodiments, a dithio-stapled (i.e., dithio-crosslinked) peptide or pharmaceutically acceptable salt thereof provided herein is of any one of SEQ ID NOs: 65-70 and 77, wherein X¹ and X² are connected via (mxy). In certain embodiments, a dithio-stapled (i.e., dithio-crosslinked) peptide or pharmaceutically acceptable salt thereof provided herein is of any one of SEQ ID NOs: 65-70 and 77, wherein X¹ and X² are connected via (but). In certain embodiments, the peptide or pharmaceutically acceptable salt thereof is of any one of SEQ ID NOs: 65-70 and 77; X¹ and X² are connected via (mxy); and the C-terminus is —CO₂H. In certain embodiments, the peptide or pharmaceutically acceptable salt thereof is of any one of SEQ ID NOs: 65-70 and 77; X¹ and X² are connected via (but); and the C-terminus is —CO₂H.

In certain embodiments, a dithio-stapled (i.e., dithio-crosslinked) peptide or pharmaceutically acceptable salt thereof is one of the following peptides:

| | | | | | | | | | | | | | | | | | | | | | | | | | | X1—X2 | |
|---|---|---|---|---|---|---|---|---|---|---|---|---|---|---|---|---|---|---|---|---|---|---|---|---|---|---|---|
| StAMP # | Sequence | | | | | | | | | | | | | | | | | | | | | | | | SEQ ID NO: | Crosslink | C-terminus |
| 2 | G | I | F | S | K | L | X¹ | G | K | K | X² | K | N | L | L | I | S | G | L | K | G | | | | 7 | mxy | —NH₂ |
| 3 | G | I | F | S | K | L | X¹ | G | K | K | X² | K | N | L | L | I | S | G | L | K | G | | | | 7 | pxy | NH₂ |
| 4 | G | I | F | S | K | L | X¹ | G | K | K | X² | K | N | L | L | I | S | G | L | K | G | | | | 7 | but | —NH₂ |
| 91 | G | I | F | S | K | L | X¹ | G | K | K | X² | K | N | L | L | I | S | G | L | K | G | | | | 7 | mxy | —CO₂H |
| 92 | G | I | F | S | K | L | X¹ | G | K | K | X² | K | N | L | L | I | S | G | L | K | G | G | G | | 74 | mxy | —CO₂H |
| 93 | G | I | F | S | K | L | X¹ | G | K | K | X² | K | N | L | L | I | S | G | L | K | G | G | G | G | 65 | mxy | CO₂H |
| 94 | G | I | F | S | K | L | X¹ | G | K | K | X² | K | N | L | L | I | S | G | L | K | G | G | G | K | 66 | mxy | CO₂H |
| 95 | G | I | F | S | K | L | X¹ | G | K | K | X² | K | N | L | L | I | S | G | L | K | G | A | G | | 67 | mxy | CO₂H |
| 96 | G | I | F | S | K | L | X¹ | G | K | K | X² | K | N | L | L | I | S | G | L | K | G | G | K | | 68 | mxy | CO₂H |
| 97 | G | I | F | S | K | L | X¹ | G | K | K | X² | K | N | L | L | I | S | G | L | K | G | | | | 69 | mxy | CO₂H |
| 98 | G | I | F | S | K | L | X¹ | G | K | K | X² | K | N | L | L | I | S | G | L | K | G | | | | 70 | mxy | CO₂H |
| 99 | G | I | F | S | K | L | X¹ | G | K | K | X² | K | N | L | L | I | S | G | L | K | G | | | | 77 | mxy | CO₂H |
| 100 | G | I | F | S | K | L | X¹ | G | K | K | X² | K | N | L | L | I | S | G | L | K | G | A | K | | 68 | but | CO₂H |

In certain embodiments, a dithio stapled (i.e., dithio crosslinked) peptide or pharmaceutically acceptable salt thereof provided herein comprises any one of SEQ ID NOs: 1-17, wherein:
X¹ and X² are independently amino acids connected via a dithio crosslink;
optionally wherein the amino acid sequence includes 1 to 6 amino acid substitutions, inclusive, at positions other than X¹ and X².

In certain embodiments, X¹ and X² in any one of SEQ ID NOs: 1-17 are connected via a dithio crosslink selected from (mxy), (pxy), (but), (bbn), (bbf), (bbp), (pfb), and (hfb). In certain embodiments, X¹ and X² in any one of SEQ ID NOs: 1-17 are connected via a dithio crosslink selected from (mxy), (pxy), and (but). In certain embodiments, X¹ and X² in any one of SEQ ID NOs: 1-17 are connected via (mxy). In certain embodiments, X¹ and X² in any one of SEQ ID NOs: 1-17 are connected via (pxy). In certain embodiments, X¹ and X² in any one of SEQ ID NOs: 1-17 are connected via (but).

In certain embodiments, a dithio-stapled (i.e., dithio-crosslinked) peptide or pharmaceutically acceptable salt thereof provided herein is of any one of SEQ ID NOs: 1-17, wherein X¹ and X² are connected via (mxy). In certain embodiments, the peptide or pharmaceutically acceptable salt thereof is of any one of SEQ ID NOs: 1-17; X¹ and X² are connected via (mxy); and the C-terminus of the peptide is amidated with —NH₂.

In certain embodiments, a dithio-stapled (i.e., dithio-crosslinked) peptide or pharmaceutically acceptable salt thereof provided herein is of any one of SEQ ID NOs: 1-17, wherein X¹ and X² are connected via (pxy). In certain embodiments, the peptide or pharmaceutically acceptable salt thereof is of any one of SEQ ID NOs: 1-17; X¹ and X² are connected via (pxy); and the C-terminus of the peptide is amidated with —NH₂.

In certain embodiments, a dithio-stapled (i.e., dithio-crosslinked) peptide or pharmaceutically acceptable salt thereof provided herein is of any one of SEQ ID NOs: 1-17, wherein X¹ and X² are connected via (but). In certain embodiments, the peptide or pharmaceutically acceptable salt thereof is of any one of SEQ ID NOs: 1-17; X¹ and X² are connected via (but); and the C-terminus of the peptide is amidated with —NH₂.

In certain embodiments, X¹ and X² in SEQ ID NO: 78 are connected via a dithio crosslink selected from (mxy), (pxy), (but), (bbn), (bbf), (bbp), (pfb), and (hfb). In certain embodiments, X¹ and X² in SEQ ID NO: 78 are connected via a dithio crosslink selected from (mxy), (pxy), and (but). In certain embodiments, X¹ and X² in SEQ ID NO: 78 are connected via (but). In certain embodiments, a dithio-stapled (i.e., dithio-crosslinked) peptide or pharmaceutically acceptable salt thereof provided herein is of SEQ ID NO: 78, wherein X¹ and X² are connected via (but). In certain embodiments, the peptide or pharmaceutically acceptable salt thereof is of SEQ ID NO: 78; X¹ and X² are connected via (but); and the C-terminus of the peptide is —COOH or amidated with —NH₂ or —NHOH.

In certain embodiments, a dithio-stapled (i.e., dithio-crosslinked) peptide or pharmaceutically acceptable salt thereof is one of the following peptides:

| StAMP | | | | | | | | | | | | | | | | | | | | | | | X1.X2 | C- |
|---|---|---|---|---|---|---|---|---|---|---|---|---|---|---|---|---|---|---|---|---|---|---|---|---|
| # | Sequence | | | | | | | | | | | | | | | | | | | | | SEQ ID NO: | Crosslink | terminus |
| 1 | G | I | X¹ | S | K | L | X² | G | K | K | I | K | N | L | L | I | S | G | L | K | G | 3 | but | —NH₂ |
| 2 | G | I | F | S | K | L | X¹ | G | K | K | X² | K | N | L | L | I | S | G | L | K | G | 7 | mxy | NH₂ |
| 3 | G | I | F | S | K | L | X¹ | G | K | K | X² | K | N | L | L | I | S | G | L | K | G | 7 | pxy | NH₂ |
| 4 | G | I | F | S | K | L | X¹ | G | K | K | X² | K | N | L | L | I | S | G | L | K | G | 7 | but | NH₂ |
| 5 | G | I | F | X¹ | K | L | A | X² | K | K | I | K | N | L | L | I | S | G | L | K | G | 4 | mxy | NH₂ |
| 6 | G | I | F | X¹ | K | L | A | X² | K | K | I | K | N | L | L | I | S | G | L | K | G | 4 | pxy | NH₂ |
| 7 | G | I | F | X¹ | K | L | A | X² | K | K | I | K | N | L | L | I | S | G | L | K | G | 4 | but | NH₂ |
| 8 | G | I | F | S | K | X¹ | A | G | K | X² | I | K | N | L | L | I | S | G | L | K | G | 6 | pxy | NH₂ |
| 9 | G | I | F | S | K | X¹ | A | G | K | X² | I | K | N | L | L | I | S | G | L | K | G | 6 | but | NH₂ |
| 10 | G | I | F | S | K | L | A | X₁ | K | K | I | X² | N | L | L | I | S | G | L | K | G | 8 | mxy | NH₂ |
| 11 | G | I | F | S | K | L | A | X₁ | K | K | I | X² | N | L | L | I | S | G | L | K | G | 8 | pxy | NH₂ |
| 12 | G | I | F | S | K | L | A | X₁ | K | K | I | X² | N | L | L | I | S | G | L | K | G | 8 | but | NH₂ |
| 13 | G | I | F | S | K | L | A | G | X¹ | K | I | K | X² | L | L | I | S | G | L | K | G | 9 | pxy | NH₂ |
| 14 | G | I | F | S | K | L | A | G | K | X¹ | I | K | N | X² | L | I | S | G | L | K | G | 10 | mxy | NH₂ |
| 15 | G | I | F | S | K | L | A | G | K | X¹ | I | K | N | X² | L | I | S | G | L | K | G | 10 | pxy | NH₂ |
| 16 | G | I | F | S | K | L | A | G | K | X¹ | I | K | N | X² | L | I | S | G | L | K | G | 10 | but | NH₂ |
| 17 | G | I | F | S | K | L | A | G | K | K | X¹ | K | N | L | X² | I | S | G | L | K | G | 11 | mxy | NH₂ |
| 18 | G | I | F | S | K | L | A | G | K | K | X¹ | K | N | L | X² | I | S | G | L | K | G | 11 | pxy | NH₂ |
| 19 | G | I | F | S | K | L | A | G | K | K | X¹ | K | N | L | X² | I | S | G | L | K | G | 11 | but | NH₂ |
| 20 | G | I | F | S | K | L | A | G | K | K | I | X¹ | N | L | L | X² | S | G | L | K | G | 12 | mxy | NH₂ |
| 21 | G | I | F | S | K | L | A | G | K | K | I | X¹ | N | L | L | X² | S | G | L | K | G | 12 | pxy | NH₂ |
| 22 | G | I | F | S | K | L | A | G | K | K | I | X¹ | N | L | L | X² | S | G | L | K | G | 12 | but | NH₂ |
| 23 | G | I | F | S | K | L | A | G | K | K | I | K | X¹ | L | L | I | X² | G | L | K | G | 13 | mxy | NH₂ |
| 24 | G | I | F | S | K | L | A | G | K | K | I | K | X¹ | L | L | I | X² | G | L | K | G | 13 | pxy | NH₂ |
| 25 | G | I | F | S | K | L | A | G | K | K | I | K | X¹ | L | L | I | X² | G | L | K | G | 13 | but | NH₂ |
| 26 | G | I | F | S | K | L | A | G | K | K | I | K | N | X¹ | L | I | S | X² | L | K | G | 14 | mxy | NH₂ |
| 27 | G | I | F | S | K | L | A | G | K | K | I | K | N | X¹ | L | I | S | X² | L | K | G | 14 | pxy | NH₂ |
| 28 | G | I | F | S | K | L | A | G | K | K | I | K | N | X¹ | L | I | S | X² | L | K | G | 14 | but | NH₂ |
| 29 | G | I | F | S | K | L | A | G | K | K | I | K | N | L | X¹ | I | S | G | X² | K | G | 15 | mxy | NH₂ |
| 30 | G | I | F | S | K | L | A | G | K | K | I | K | N | L | X¹ | I | S | G | X² | K | G | 15 | pxy | NH₂ |
| 31 | G | I | F | S | K | L | A | G | K | K | I | K | N | L | X¹ | I | S | G | X² | K | G | 15 | but | NH₂ |
| 32 | G | I | F | S | K | L | A | G | K | K | I | K | N | L | L | X¹ | S | G | L | X² | G | 16 | mxy | NH₂ |
| 33 | G | I | F | S | K | L | A | G | K | K | I | K | N | L | L | X¹ | S | G | L | X² | G | 16 | pxy | NH₂ |
| 34 | G | I | F | S | K | L | A | G | K | K | I | K | N | L | L | X¹ | S | G | L | X² | G | 16 | but | NH₂ |
| 35 | G | I | F | S | K | L | A | G | K | K | I | K | N | L | L | I | X¹ | G | L | K | X² | 17 | but | NH₂ |
| 36 | X¹ | I | F | S | X² | L | A | G | K | K | I | K | N | L | L | I | S | G | L | K | G | 1 | mxy | NH₂ |
| 37 | X¹ | I | F | S | X² | L | A | G | K | K | I | K | N | L | L | I | S | G | L | K | G | 1 | pxy | NH₂ |
| 38 | X¹ | I | F | S | X² | L | A | G | K | K | I | K | N | L | L | I | S | G | L | K | G | 1 | but | NH₂ |
| 39 | G | I | X¹ | S | K | L | X² | G | K | K | I | K | N | L | L | I | S | G | L | K | G | 3 | mxy | NH₂ |
| 40 | G | I | X¹ | S | K | L | X² | G | K | K | I | K | N | L | L | I | S | G | L | K | G | 3 | pxy | NH₂ |
| 41 | G | I | F | S | K | X¹ | A | G | K | X² | I | K | N | L | L | I | S | G | L | K | G | 6 | mxy | NH₂ |
| 42 | G | I | F | S | K | L | A | G | X¹ | K | I | K | X² | L | L | I | S | G | L | K | G | 9 | mxy | NH₂ |
| 43 | G | I | F | S | K | L | A | G | X¹ | K | I | K | X² | L | L | I | S | G | L | K | G | 9 | but | NH₂ |
| 44 | G | I | F | S | K | L | A | G | K | K | I | K | N | L | L | I | X¹ | G | L | K | X² | 17 | mxy | NH₂ |
| 45 | G | I | F | S | K | L | A | G | K | K | I | K | N | L | L | I | X¹ | G | L | K | X² | 17 | pxy | NH₂ |
| 46 | G | X¹ | F | S | K | X² | A | G | K | K | I | K | N | L | L | I | S | G | L | K | G | 2 | mxy | NH₂ |
| 47 | G | X¹ | F | S | K | X² | A | G | K | K | I | K | N | L | L | I | S | G | L | K | G | 2 | pxy | NH₂ |
| 48 | G | X¹ | F | S | K | X² | A | G | K | K | I | K | N | L | L | I | S | G | L | K | G | 2 | but | NH₂ |
| 101 | J | I | F | S | K | X² | A | G | K | X² | I | K | N | L | L | I | S | G | L | K | G | 78 | but | CO₂H |
| 102 | J | I | F | S | K | X² | A | G | K | X² | I | K | N | L | L | I | S | G | L | K | G | 78 | but | NH₂ |
| 103 | J | I | F | S | K | X² | A | G | K | X² | I | K | N | L | L | I | S | G | L | K | G | 78 | but | NHOH |

In certain embodiments, a dithio stapled (i.e., dithio crosslinked) peptide or pharmaceutically acceptable salt thereof provided herein comprises any one of SEQ ID NOs: 18-31, wherein:
X¹ and X² are independently amino acids connected via a dithio crosslink;
optionally wherein the amino acid sequence includes 1 to 6 amino acid substitutions, inclusive, at positions other than X¹ and X².

In certain embodiments, X¹ and X² in any one of SEQ ID NOs: 18-31 are connected via a dithio crosslink selected from (mxy), (pxy), (but), (bbn), (bbf), (bbp), (pfb), and (hfb). In certain embodiments, X¹ and X² in any one of SEQ ID NOs: 18-31 are connected via a dithio crosslink selected from (bbn), (bbf), and (bbp). In certain embodiments, X¹ and X² in any one of SEQ ID NOs: 18-31 are connected via (bbn). In certain embodiments, X¹ and X² in any one of SEQ ID NOs: 18-31 are connected via (bbf). In certain embodiments, X¹ and X² in any one of SEQ ID NOs: 18-31 are connected via (bbp).

In certain embodiments, a dithio-stapled (i.e., dithio-crosslinked) peptide or pharmaceutically acceptable salt thereof provided herein is of any one of SEQ ID NOs: 18-31, wherein X¹ and X² are connected via (bbn). In certain embodiments, the peptide or pharmaceutically acceptable salt thereof is of any one of SEQ ID NOs: 18-31; X¹ and X² are connected via (bbn); and the C-terminus of the peptide is amidated with —NH₂.

In certain embodiments, a dithio-stapled (i.e., dithio-crosslinked) peptide or pharmaceutically acceptable salt thereof provided herein is of any one of SEQ ID NOs: 18-31, wherein X¹ and X² are connected via (bbf). In certain embodiments, the peptide or pharmaceutically acceptable salt thereof is of any one of SEQ ID NOs: 18-31; X¹ and X² are connected via (bbf); and the C-terminus of the peptide is amidated with —NH₂.

In certain embodiments, a dithio-stapled (i.e., dithio-crosslinked) peptide or pharmaceutically acceptable salt thereof provided herein is of any one of SEQ ID NOs: 18-31, wherein X¹ and X² are connected via (bbp). In certain embodiments, the peptide or pharmaceutically acceptable salt thereof is of any one of SEQ ID NOs: 18-31; X¹ and X² are connected via (bbp); and the C-terminus of the peptide is amidated with —NH₂.

In certain embodiments, a dithio-stapled (i.e., dithio-crosslinked) peptide or pharmaceutically acceptable salt thereof is one of the following peptides:

| | | | | | | | | | | | | | | | | | | | | | | SEQ | X1—X2 | C- |
|---|---|---|---|---|---|---|---|---|---|---|---|---|---|---|---|---|---|---|---|---|---|---|---|---|
| StAMP # | Sequence | | | | | | | | | | | | | | | | | | | | | ID NO: | Crosslink | terminus |
| 49 | X¹ | I | F | S | K | L | A | X² | K | K | I | K | N | L | L | I | S | G | L | K | G | 18 | bbp | —NH₂ |
| 50 | X¹ | I | F | S | K | L | A | X² | K | K | I | K | N | L | L | I | S | G | L | K | G | 18 | bbn | —NH₂ |
| 51 | X¹ | I | F | S | K | L | A | X² | K | K | I | K | N | L | L | I | S | G | L | K | G | 18 | bbf | —NH₂ |
| 52 | G | X¹ | F | S | K | L | A | G | X² | K | I | K | N | L | L | I | S | G | L | K | G | 19 | bbn | —NH₂ |
| 53 | G | X¹ | F | S | K | L | A | G | X² | K | I | K | N | L | L | I | S | G | L | K | G | 19 | bbf | —NH₂ |
| 54 | G | I | X¹ | S | K | L | A | G | K | X² | I | K | N | L | L | I | S | G | L | K | G | 20 | bbp | —NH₂ |
| 55 | G | I | X¹ | S | K | L | A | G | K | X² | I | K | N | L | L | I | S | G | L | K | G | 20 | bbn | —NH₂ |
| 56 | G | I | X¹ | S | K | L | A | G | K | X² | I | K | N | L | L | I | S | G | L | K | G | 20 | bbf | —NH₂ |
| 57 | G | I | F | X¹ | K | L | A | G | K | K | X² | K | N | L | L | I | S | G | L | K | G | 21 | bbp | —NH₂ |
| 58 | G | I | F | X¹ | K | L | A | G | K | K | X² | K | N | L | L | I | S | G | L | K | G | 21 | bbn | —NH₂ |
| 59 | G | I | F | X¹ | K | L | A | G | K | K | X² | K | N | L | L | I | S | G | L | K | G | 21 | bbf | —NH₂ |
| 60 | G | I | F | S | X¹ | L | A | G | K | I | I | X² | N | L | L | I | S | G | L | K | G | 22 | bbp | —NH₂ |
| 61 | G | I | F | S | X¹ | L | A | G | K | I | I | X² | N | L | L | I | S | G | L | K | G | 22 | bbf | —NH₂ |
| 62 | G | I | F | S | X¹ | L | A | G | K | I | I | X² | N | L | L | I | S | G | L | K | G | 22 | bbn | —NH₂ |
| 63 | G | I | F | S | K | X¹ | A | G | K | K | I | K | X² | L | L | I | S | G | L | K | G | 23 | bbp | —NH₂ |
| 64 | G | I | F | S | K | X¹ | A | G | K | K | I | K | X² | L | L | I | S | G | L | K | G | 23 | bbn | —NH₂ |
| 65 | G | I | F | S | K | X¹ | A | G | K | K | I | K | X² | L | L | I | S | G | L | K | G | 23 | bbf | —NH₂ |
| 66 | G | I | F | S | K | L | A | X¹ | K | K | I | K | N | L | X² | I | S | G | L | K | G | 24 | bbp | —NH₂ |
| 67 | G | I | F | S | K | L | A | X¹ | K | K | I | K | N | L | X² | I | S | G | L | K | G | 24 | bbn | —NH₂ |
| 68 | G | I | F | S | K | L | A | X¹ | K | K | I | K | N | L | X² | I | S | G | L | K | G | 24 | bbf | —NH₂ |
| 69 | G | I | F | S | K | L | A | G | X¹ | K | I | K | N | L | L | X² | S | G | L | K | G | 25 | bbn | —NH₂ |
| 70 | G | I | F | S | K | L | A | G | X¹ | K | I | K | N | L | L | X² | S | G | L | K | G | 25 | bbf | —NH₂ |
| 71 | G | I | F | S | K | L | A | G | X¹ | K | I | K | N | L | L | X² | S | G | L | K | G | 25 | bbp | —NH₂ |
| 72 | G | I | F | S | K | L | A | G | K | X¹ | I | K | N | L | L | I | X² | G | L | K | G | 26 | bbp | —NH₂ |
| 73 | G | I | F | S | K | L | A | G | K | X¹ | I | K | N | L | L | I | X² | G | L | K | G | 26 | bbn | —NH₂ |
| 74 | G | I | F | S | K | L | A | G | K | X¹ | I | K | N | L | L | I | X² | G | L | K | G | 26 | bbf | —NH₂ |
| 75 | G | I | F | S | K | L | A | G | K | K | X¹ | K | N | L | L | I | S | X² | L | K | G | 27 | bbn | —NH₂ |
| 76 | G | I | F | S | K | L | A | G | K | K | X¹ | K | N | L | L | I | S | X² | L | K | G | 27 | bbf | —NH₂ |
| 77 | G | I | F | S | K | L | A | G | K | K | X¹ | K | N | L | L | I | S | X² | L | K | G | 27 | bbp | —NH₂ |
| 78 | G | I | F | S | K | L | A | G | K | K | I | X¹ | N | L | L | I | S | G | X² | K | G | 28 | bbn | —NH₂ |
| 79 | G | I | F | S | K | L | A | G | K | K | I | X¹ | N | L | L | I | S | G | X² | K | G | 28 | bbf | —NH₂ |
| 80 | G | I | F | S | K | L | A | G | K | K | I | C | N | L | L | I | S | G | C | K | G | 28 | bbp | —NH₂ |
| 81 | G | I | F | S | K | L | A | G | K | K | I | K | X¹ | L | L | I | S | G | L | X² | G | 29 | bbp | —NH₂ |
| 82 | G | I | F | S | K | L | A | G | K | K | I | K | X¹ | L | L | I | S | G | L | X² | G | 29 | bbn | —NH₂ |
| 83 | G | I | F | S | K | L | A | G | K | K | I | K | X¹ | L | L | I | S | G | L | X² | G | 29 | bbf | —NH₂ |
| 84 | G | I | F | S | K | L | A | G | K | K | I | K | N | X¹ | L | I | S | G | L | K | X² | 30 | bbp | —NH₂ |
| 85 | G | I | F | S | K | L | A | G | K | K | I | K | N | X¹ | L | I | S | G | L | K | X² | 30 | bbn | —NH₂ |
| 86 | G | I | F | S | K | L | A | G | K | K | I | K | N | X¹ | L | I | S | G | L | K | X² | 30 | bbf | —NH₂ |
| 87 | G | I | F | S | K | L | X¹ | G | K | K | I | K | N | X² | L | I | S | G | L | K | G | 31 | bbn | —NH₂ |
| 88 | G | I | F | S | K | L | X¹ | G | K | K | I | K | N | X² | L | I | S | G | L | K | G | 31 | bbf | —NH₂ |
| 89 | G | I | F | S | K | L | X¹ | G | K | K | I | K | N | X² | L | I | S | G | L | K | G | 31 | bbp | —NH₂ |

In other embodiments, a dithio stapled (i.e., dithio crosslinked) peptide provided herein comprises the following amino acid sequence:

(SEQ ID NO: 32)
G I F S K L X¹ G K K X² K N L L I X³ G L K X⁴,

or a pharmaceutically acceptable salt thereof, wherein:
X¹, X², X³, and X⁴ are independently amino acids (i.e., dithio crosslinked amino acids);
X¹ and X² are connected via a dithio crosslink, and X³ and X⁴ are connected via a dithio crosslink; and
optionally wherein the amino acid sequence includes 1 to 6 amino acid substitutions, inclusive, at positions other than X¹, X², X³, and X⁴.

In certain embodiments, X¹ and X², and X³ and X⁴, in SEQ ID NO: 32 are connected via dithio crosslinks independently selected from (mxy), (pxy), (but), (bbn), (bbf), (bbp), (pfb), and (hfb). In certain embodiments, X¹ and X², and X³ and X⁴, in SEQ ID NO: 32 are connected via dithio crosslinks independently selected from (mxy), (pxy), and (but). In certain embodiments, X¹ and X², and X³ and X⁴, in SEQ ID NO: 32 are connected via (mxy). In certain embodiments, X¹ and X², and X³ and X⁴, in SEQ ID NO: 32 are connected via (pxy). In certain embodiments, X¹ and X², and X³ and X⁴, in SEQ ID NO: 32 are connected via (but).

In certain embodiments, a dithio-stapled (i.e., dithio-crosslinked) peptide or pharmaceutically acceptable salt thereof provided herein is of SEQ ID NO: 32, wherein X¹ and X² are connected via (pxy). In certain embodiments, the peptide or pharmaceutically acceptable salt thereof is of SEQ ID NO: 32; X¹ and X² are connected via (pxy); and the C-terminus of the peptide is amidated with —NH₂.

In certain embodiments, a dithio-stapled (i.e., dithio-crosslinked) peptide or pharmaceutically acceptable salt thereof is the following peptide (the C-terminus of StAMP 90 is amidated with —NH₂):

| | | | | | | | | | | | | | | | | | | | | | | | X1—X2 and | |
|---|---|---|---|---|---|---|---|---|---|---|---|---|---|---|---|---|---|---|---|---|---|---|---|---|
| | | | | | | | | | | | | | | | | | | | | | | | X3—X4 | |
| StAMP # | Sequence | | | | | | | | | | | | | | | | | | | | | SEQ ID NO: | Crosslink | C-terminus |
| 90 | G | I | F | S | K | L | X¹ | G | K | K | X² | K | N | L | L | I | X³ | G | L | K | X⁴ | 32 | pxy | —NH₂ |

In addition to pharmaceutically acceptable salts of the stapled peptides provided herein, also provided herein are stereoisomers, tautomers, isotopically labeled derivatives, solvates, hydrates, polymorphs, co-crystals, and prodrugs of the stapled peptides provided herein.

As described herein, stapling (e.g., crosslinking) a peptide can stabilize a secondary structure (e.g., α-helical secondary structure) of the peptide. In certain embodiments, one or more crosslinks of a stapled peptide provided herein stabilize an α-helix of the peptide. In certain embodiments, a peptide has increased α-helicity as compared to a corresponding unstapled (e.g., uncrosslinked) peptide.

A stapled peptide provided herein can exhibit α-helical stability by the maintenance of α-helical structure as measured by circular dichroism or NMR. For example, in certain embodiments, the stapled peptide exhibits at least a 1.1, 1.2, 1.25, 1.3, 1.4, 1.5, 1.6, 1.7, 1.75, 1.8, 1.9, or 2-fold increase in α-helicity (e.g., as determined by circular dichroism or NMR) compared to a corresponding unstapled peptide. In certain embodiments, a stapled peptide provided herein can exhibit about 10%, 15%, 20%, 25%, 30%, 35%, 40%, 45%, 50%, 55%, 60%, 65%, 70%, 75%, 80%, 85%, 90%, 95%, or 100% α-helicity (e.g., as determined by circular dichroism or NMR) compared to a corresponding unstapled peptide.

### Stapled Peptide Crosslinks

The stapled peptides provided herein comprise crosslinks (e.g., staples), wherein each crosslink connects two amino acids (i.e., crosslinked amino acids) to form a macrocycle. In certain embodiments, when an amino acid sequence comprises X¹ and X², X¹ and X² are crosslinked amino acids connected via a crosslink. Likewise, in certain embodiments, when an amino acid sequence comprises X³ and X⁴, X³ and X⁴ are crosslinked amino acids connected via a crosslink.

In certain embodiments, the crosslinks are independently attached to the α-positions of the crosslinked amino acids (e.g., α-positions of X¹, X², X³, and/or X⁴). In certain embodiments, the crosslinks are independently attached to the α-positions of the crosslinked amino acids (e.g., X¹, X², X³, and/or X⁴), and the crosslinked amino acids are independently α,α-disubstituted amino acids.

In certain embodiments, each crosslink is independently from about 5 Å to about 35 Å in length, inclusive.

In certain embodiments, each crosslink is independently from about 5 Å to about 25 Å in length, inclusive (e.g., in the case of i+4 crosslinks). In certain embodiments, each crosslink is independently from about 6 Å to about 22 Å in length, inclusive. In certain embodiments, each crosslink is independently from about 7 Å to about 20 Å in length, inclusive. In certain embodiments, each crosslink is independently from about 8 Å to about 18 Å in length, inclusive. In certain embodiments, each crosslink is independently from about 9 Å to about 17 Å in length, inclusive. Each crosslink is independently about 10 Å to about 16 Å in length, inclusive. In certain embodiments, each crosslink is independently from about 11 Å to about 15 Å in length, inclusive. In certain embodiments, each crosslink is independently from about 12 Å to about 14 Å in length, inclusive. In certain embodiments, each crosslink is independently about 5, 6, 7, 8, 9, 10, 11, 12, 13, 14, 15, 16, 17, 18, 19, 20, 21, 22, 23, 24, or 25 Å in length.

In certain embodiments, each crosslink is independently from about 15 Å to about 35 Å in length, inclusive (e.g., in the case of i+7 crosslinks). In certain embodiments, each crosslink is independently from about 17 Å to about 33 Å in length, inclusive. In certain embodiments, each crosslink is independently from about 19 Å to about 31 Å in length, inclusive. In certain embodiments, each crosslink is independently from about 20 Å to about 30 Å in length, inclusive. In certain embodiments, each crosslink is independently from about 22 Å to about 29 Å in length, inclusive. Each crosslink is independently about 24 Å to about 28 Å in length, inclusive. In certain embodiments, each crosslink is independently from about 25 Å to about 27 Å in length, inclusive. In certain embodiments, each crosslink is independently about 15, 16, 17, 18, 19, 20, 21, 22, 23, 24, 25, 26, 27, 28, 29, 30, 31, 32, 33, 34, or 35 in length.

In certain embodiments, the length of each crosslink is approximately equal to the length of 5 to 25 carbon-carbon and/or carbon-sulfur bonds, inclusive. In certain embodiments, the length of each crosslink is approximately equal to the length of 5, 6, 7, 8, 9, 10, 11, 12, 13, 14, 15, 16, 17, 18, 19, 20, 21, 22, 23, 24, or 25 carbon-carbon and/or carbon-sulfur bonds, inclusive.

In certain embodiments, the length of each crosslink is approximately equal to the length of 5 to 20 carbon-carbon and/or carbon-sulfur bonds, inclusive. In certain embodiments, the length of each crosslink is approximately equal to the length of 5 to 15 carbon-carbon and/or carbon-sulfur bonds, inclusive. In certain embodiments, the length of each crosslink is approximately equal to the length of 5 to 13 carbon-carbon and/or carbon-sulfur bonds, inclusive. In certain embodiments, the length of each crosslink is approximately equal to the length of 6 to 12 carbon-carbon and/or carbon-sulfur bonds, inclusive. In certain embodiments, the length of each crosslink is approximately equal to the length of 7 to 11 carbon-carbon and/or carbon-sulfur bonds, inclusive. In certain embodiments, the length of each crosslink is approximately equal to the length of 8 to 10 carbon-carbon and/or carbon-sulfur bonds, inclusive.

In certain embodiments, the length of each crosslink is approximately equal to the length of 10 to 20 carbon-carbon and/or carbon-sulfur bonds, inclusive. In certain embodiments, the length of each crosslink is approximately equal to the length of 11 to 19 carbon-carbon and/or carbon-sulfur bonds, inclusive. In certain embodiments, the length of each crosslink is approximately equal to the length of 12 to 18 carbon-carbon and/or carbon-sulfur bonds, inclusive. In certain embodiments, the length of each crosslink is approximately equal to the length of 13 to 17 carbon-carbon and/or carbon-sulfur bonds, inclusive. In certain embodiments, the length of each crosslink is approximately equal to the length of 14 to 16 carbon-carbon and/or carbon-sulfur bonds, inclusive.

In certain embodiments, at least one crosslink spans at least one turn of an α-helix of the peptide. In certain embodiments, each crosslink spans at least one turn of an α-helix of the peptide. In certain embodiments, at least one crosslink spans one turn of an α-helix of the peptide. In certain embodiments, each crosslink spans one turn of an α-helix of the peptide.

In certain embodiments, each pair of crosslinked amino acids (e.g., X¹ and X², and X³ and X⁴) are independently connected by a crosslink to form the following formula:

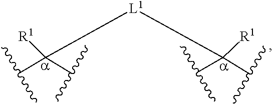

wherein α denotes the α-carbons of the crosslinked amino acids; L¹ is a crosslink; and each instance of R¹ is independently hydrogen or optionally substituted C₁₋₆ alkyl.

In certain embodiments, each crosslink (e.g., L¹) is independently optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

In certain embodiments, L¹ is a dithio crosslink.

### Dithio Crosslinks

In certain embodiments, a crosslink (e.g., L¹) is independently a dithio crosslink. For the purposes of this disclosure, a “dithio crosslink” (i.e., “dithio staple”) is a crosslink comprising two thioethers (i.e., two —S— groups). In certain embodiments, a crosslink is independently a dithio crosslink of the following formula:

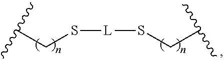

wherein each n is independently an integer from 1-10, inclusive; and
L is optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof. In certain embodiments, each instance of n is 1. In certain embodiments, each instance of n is 2.

In certain embodiments, a crosslink is independently a dithio crosslink of the following formula:

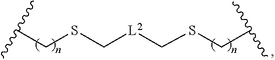

wherein each n is independently an integer from 1-10, inclusive; and
L² is optionally substituted alkylene,

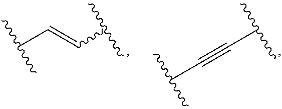

optionally substituted arylene, optionally substituted heteroarylene, or -A¹-A¹-; wherein each instance of A¹ is independently optionally substituted arylene or optionally substituted heteroarylene. In certain embodiments, each instance of n is 1. In certain embodiments, each instance of n is 2.

In certain embodiments, a crosslink is independently a dithio crosslink of one of the following formulae:

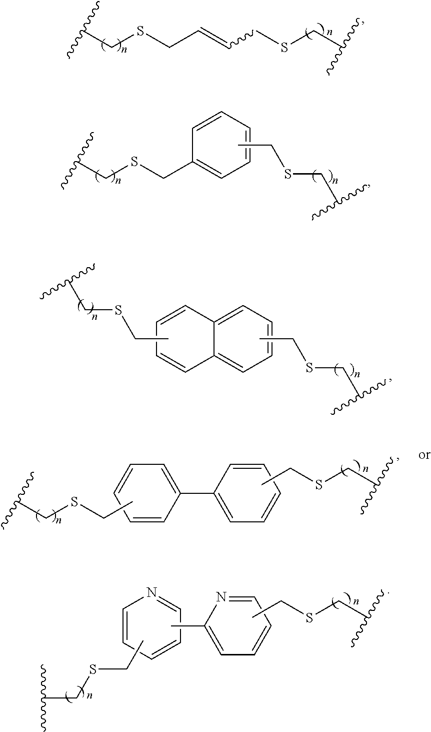

In certain embodiments, each instance of n is 1. In certain embodiments, each instance of n is 2.

For example, in certain embodiments, a crosslink is independently a dithio crosslink of one of the following formulae:

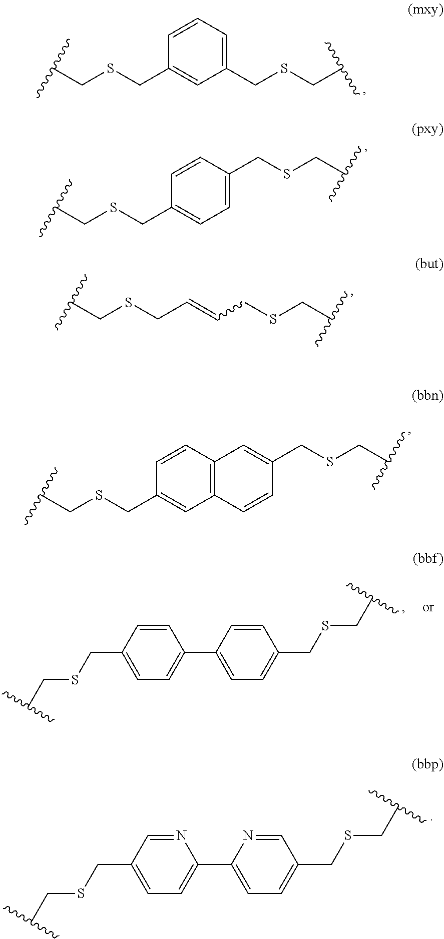 [SMILES: *CSCc1cccc(CSC*)c1 ; *CSCc1ccc(CSC*)cc1 ; *CSCC=CCSC* ; *CSCc1ccc2cc(CSC*)ccc2c1 ; *CSCc1ccc(-c2ccc(CSC*)cc2)cc1 ; *CSCc1ccc(-c2ccc(CSC*)cn2)nc1]

In other embodiments, a crosslink is independently a dithio crosslink of the following formula:

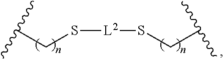

wherein each n is independently an integer from 1-10, inclusive;
L² is an optionally substituted aromatic ring (e.g., a polyhalogenated aryl or heteroaryl ring) or -A¹-A¹-; wherein each instance of A¹ is independently an optionally substituted aromatic ring (e.g., a polyhalogenated aryl or heteroaryl ring). In certain embodiments, each instance of n is 1. In certain embodiments, each instance of n is 2.

In certain embodiments, a crosslink is independently a dithio crosslink of one of the following formulae:

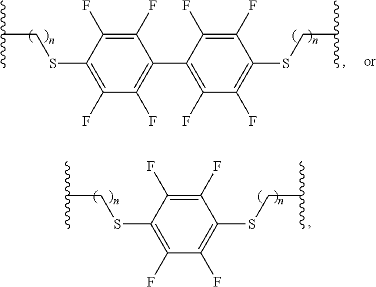

Wherein each n is independently an integer from 1-10, inclusive. In certain embodiments, each instance of n is 1. In certain embodiments, each instance of n is 2.

For example, in certain embodiments, a crosslink is independently of one of the following formulae:

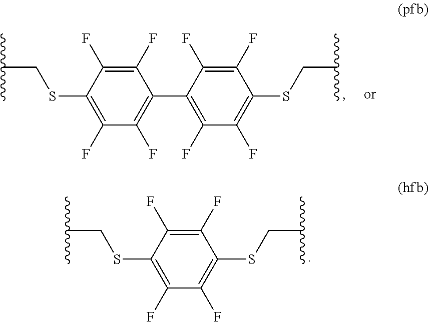 [SMILES: *CSc1c(F)c(F)c(-c2c(F)c(F)c(SC*)c(F)c2F)c(F)c1F ; *CSc1c(F)c(F)c(SC*)c(F)c1F]

For the purposes of this disclosure, the particular crosslinks (mxy), (pxy), (but), (bbn), (bbf), (bbp), (pfb), and (hfb) referenced herein are formed by crosslinking two cysteine (C) residues of a peptide. In other words, a peptide or pharmaceutically acceptable salt thereof provided herein comprising a (mxy), (pxy), (but), (bbn), (bbf), (bbp), (pfb), and/or (hfb) crosslink includes each pair of crosslinked amino acids (e.g., X¹ and X², X³ and X⁴) connected via a dithio crosslink to form the following formula:

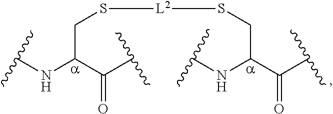

wherein each α represents the alpha-position of a dithio-crosslinked amino acid (e.g., X¹, X², X³, X⁴), and L² is as indicated in Table 2 below.

TABLE 2
Certain Dithio Crosslinks

| Dithio Crosslink | L² |
|---|---|
| mxy | 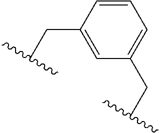 [SMILES: *Cc1cccc(C*)c1] |
| pxy | 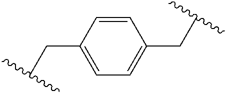 [SMILES: *Cc1ccc(C*)cc1] |
| but | 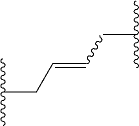 [SMILES: *CC=CC*] |
| bbn | 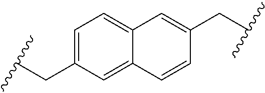 [SMILES: *Cc1ccc2cc(C*)ccc2c1] |
| bbf | 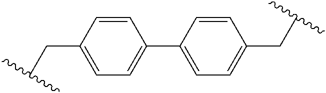 [SMILES: *Cc1ccc(-c2ccc(C*)cc2)cc1] |
| bbp | 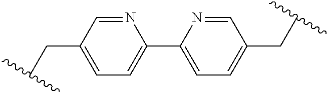 [SMILES: *Cc1ccc(-c2ccc(C*)cn2)nc1] |
| pfb | 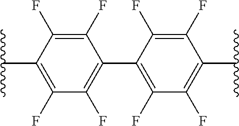 [SMILES: *c1c(F)c(F)c(-c2c(F)c(F)c(*)c(F)c2F)c(F)c1F] |
| hfb | 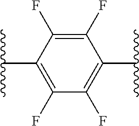 [SMILES: *c1c(F)c(F)c(*)c(F)c1F] |

### Other Crosslinks

In certain embodiments, each crosslink (e.g., L¹) is independently a hydrocarbon crosslink. “Hydrocarbon crosslink” for the purposes of this disclosure is a crosslink consisting of optionally substituted alkylene, optionally substituted alkenylene, optionally substituted alkynylene, and combinations thereof.

In certain embodiments, each crosslink (e.g., L¹) is independently optionally substituted alkenylene (e.g., unsubstituted alkenylene). In certain embodiments, each crosslink is independently of the following formula:

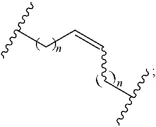

wherein each n is independently an integer from 1-10, inclusive. In certain embodiments, the sum of two n on the same crosslink is 6.

In certain embodiments, the crosslinked amino acids (e.g., X¹, X², X³, and X⁴) are independently α,α-disubstituted amino acids. For instance, in certain embodiments, each pair of crosslinked amino acids (e.g., X¹ and X², and X³ and X⁴) are independently connected by a crosslink to form the following formula:

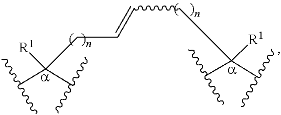

wherein α denotes the α-carbons of the crosslinked amino acids; and wherein each instance of R¹ is independently optionally substituted C₁₋₆ alkyl. In certain embodiments, the sum of two n on the same crosslink is 6.

For example, in certain embodiments, a crosslink (e.g., L¹) is independently of the formula:

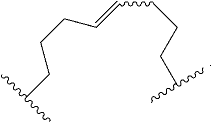 [SMILES: *CCCC=CCCC*]

For example, in certain embodiments, a pair of crosslinked amino acids (e.g., X¹ and X², and X³ and X⁴) are independently connected via a crosslink to form the following formula:

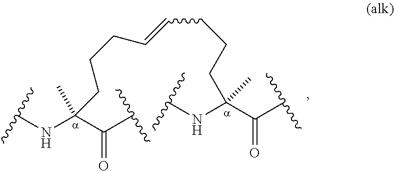 [SMILES: *N[C@@](C)(CCCC=CCCC[C@](C)(N*)C(*)=O)C(*)=O]

wherein α denotes the α-carbons of the crosslinked amino acids.

In certain embodiments, X¹ and X² are connected to form the formula (alk).

In certain embodiments, X³ and X⁴ are connected to form the formula (alk).

In certain embodiments, a crosslink (e.g., L¹) is independently optionally substituted alkylene (e.g., unsubstituted alkylene). In certain embodiments, each crosslink is independently of the following formula:

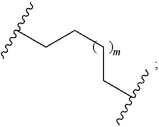

wherein m is an integer from 1-20, inclusive. In certain embodiments, m is 6.

In certain embodiments, a pair of crosslinked amino acids (e.g., X¹ and X², and X³ and X⁴) are independently joined by a crosslink to form the following formula:

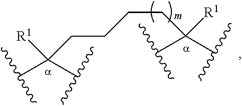

wherein α denotes the α-carbons of the crosslinked amino acids; and wherein each instance of R¹ is independently optionally substituted C₁₋₆ alkyl. In certain embodiments, m is 6.

For example, in certain embodiments, a crosslink (e.g., L¹) is independently of the formula:

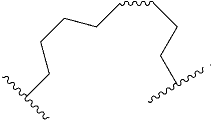 [SMILES: *CCCCCCCC*]

For example, in certain embodiments, a pair of crosslinked amino acids (e.g., X¹ and X², and X³ and X⁴) are connected via a crosslink to form the following formula:

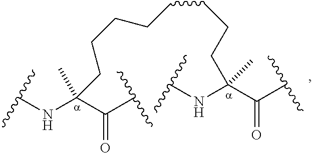 [SMILES: *N[C@@](C)(CCCCCCCC[C@](C)(N*)C(*)=O)C(*)=O]

wherein α denotes the α-carbons of the crosslinked amino acids.

In certain embodiments, a crosslink (e.g., L¹) is independently optionally substituted alkynylene (e.g., unsubstituted alkynylene).

In certain embodiments, a crosslink (e.g., L¹) is independently a triazolylene crosslink. For the purpose of this disclosure, a “triazolylene crosslink” is a crosslink interrupted by at least one triazolylene moiety

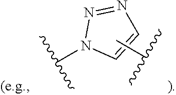

In certain embodiments, a crosslink is independently a triazolylene crosslink of the following formula:

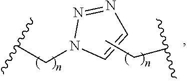

wherein each n is independently an integer from 1-10, inclusive. In certain embodiments, the sum of two n on the same crosslink is 5.

For example, in certain embodiments, a crosslink is independently a triazolylene crosslink of one of the following formulae:

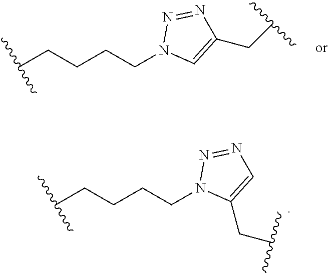 [SMILES: *CCCCn1cc(C*)nn1 ; *CCCCn1nncc1C*]

The following embodiments for n and R¹ apply to all generic formulae and subgenera provided herein, as well as all stapled and unstapled peptides provided herein.

In certain embodiments, the sum of two n on the same crosslink is an integer from 3-9, inclusive. In certain embodiments, the sum of two n on the same crosslink is an integer from 4-8, inclusive. In certain embodiments, the sum of two n on the same crosslink is an integer from 5-7, inclusive. In certain embodiments, the sum of two n on the same crosslink is 5. In certain embodiments, the sum of two n on the same crosslink is 6. In certain embodiments, the sum of two n on the same crosslink is 7.

In certain embodiments, at least one instance of n is 1. In certain embodiments, at least one instance of n is 2. In certain embodiments, at least one instance of n is 3. In certain embodiments, at least one instance of n is 4. In certain embodiments, at least one instance of n is 5. In certain embodiments, at least one instance of n is 6. In certain embodiments, at least one instance of n is 7. In certain embodiments, at least one instance of n is 8. In certain embodiments, at least one instance of n is 9. In certain embodiments, at least one instance of n is 10.

In certain embodiments, m is an integer from 3-9, inclusive. In certain embodiments, m is an integer from 4-8, inclusive. In certain embodiments, m is an integer from 5-7, inclusive. In certain embodiments, m is 5. In certain embodiments, m is 6. In certain embodiments, m is 7.

In certain embodiments, at least one instance of R¹ is hydrogen. In certain embodiments, each instance of R¹ is hydrogen. In certain embodiments, at least one instance of R¹ is unsubstituted C₁₋₆ alkyl. In certain embodiments, at least one instance of R¹ is unsubstituted C₁₋₃ alkyl. In certain embodiments, at least one instance of R¹ is methyl. In certain embodiments, each instance of R¹ is methyl.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Additional Amino Acid Substitutions

Another aspect of the present disclosure relates to the discovery that replacing lysine (K) and/or phenylalanine (F) residues of amino acid sequences recited herein with modified variants can help confer advantageous properties (e.g., improved antimicrobial activity, reduced toxicity, etc.). For example, it has been found that replacing one or more lysine (K) residues with “shorter lysine” residues (e.g., amino acids Orn, Dab, and Dap, shown below in Table 3) can help reduce toxicity (e.g., renal toxicity) of the peptides.

TABLE 3
“Shorter Lysine” Amino Acids

| | | |
|---|---|---|
| 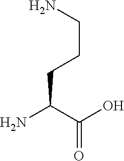 | 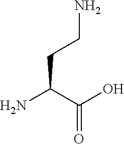 | 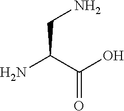 |
| Orn | Dab | Dap |

Therefore, also provided herein are peptides and pharmaceutically acceptable salts thereof comprising any amino acid sequence provided herein (e.g., SEQ ID NOs: 1-32, 65-70, 74-78), optionally wherein one or more instances of K are independently substituted by an amino acid selected from Orn, Dab, Dap, R, and hArg. In certain embodiments, one or more instances of K (e.g., 1, 2, 3, 4, 5, 6, 7, 8, 9 instances of K) are independently substituted by an amino acid selected from Orn, Dab, and Dap. In certain embodiments, one or more instances of K (e.g., 1, 2, 3, 4, 5, 6, 7, 8, 9 instances of K) are substituted by Orn. In certain embodiments, one or more instances of K (e.g., 1, 2, 3, 4, 5, 6, 7, 8, 9 instances of K) are substituted by Dab. In certain embodiments, one or more instances of K (e.g., 1, 2, 3, 4, 5, 6, 7, 8, 9 instances of K) are substituted by Dap. In certain embodiments, one or more instances of K (e.g., 1, 2, 3, 4, 5, 6, 7, 8, 9 instances of K) are substituted by R. In certain embodiments, one or more instances of K (e.g., 1, 2, 3, 4, 5, 6, 7, 8, 9 instances of K) are substituted by and hArg.

In certain embodiments, each instance of K is independently substituted by an amino acid selected from Orn, Dab, Dap, R, and hArg. In certain embodiments, each instance of K is substituted by Orn. In certain embodiments, each instance of K is substituted by Dab. In certain embodiments, each instance of K is substituted by Dap. In certain embodiments, each instance of K is substituted by R. In certain embodiments, each instance of K is substituted by hArg.

Also provided herein are peptides and pharmaceutically acceptable salts thereof comprising any amino acid sequence provided herein (e.g., SEQ ID NOs: 1-32, 65-70, 74-78), optionally wherein one or more instances of F (e.g., each instance of F) are independently substituted by a modified phenylalanine. “Modified phenylalanine” for the purpose of this disclosure means a stereoisomer of phenylalanine (e.g., D-Phe) or any unnatural phenylalanine analog wherein the phenyl ring is ortho-, meta-, and/or para-substituted with one or more non-hydrogen substituents (e.g., optionally substituted alkyl (e.g., Me, Et, n-Pr, i-Pr, n-Bu, t-Bu), halogen (e.g., F, Cl, Br, I), haloalkyl (e.g., —CF₃), optionally substituted hydroxyl (e.g., —OH), optionally substituted amino (e.g., —NH₂), optionally substituted thio (e.g., —SH), optionally substituted acyl (e.g., —C(═O)Me, —C(═O)OH, —C(═O)NH₂), —CN, —SCN, —NO₃, —N₃). In certain embodiments, one or more instances of F are independently substituted by a modified phenylalanine. In certain embodiments, each instance of F is independently substituted by a modified phenylalanine.

In certain embodiments, one or more instances of F (e.g., 1, 2, 3 instances of F) are independently substituted by an amino acid selected from F¹, F², F³, F⁴, and F⁵. In certain embodiments, one or more instances of F (e.g., 1, 2, 3 instances of F) are independently substituted by F¹. In certain embodiments, one or more instances of F (e.g., 1, 2, 3 instances of F) are independently substituted by F². In certain embodiments, one or more instances of F (e.g., 1, 2, 3 instances of F) are independently substituted by F³. In certain embodiments, one or more instances of F (e.g., 1, 2, 3 instances of F) are independently substituted by F⁴. In certain embodiments, one or more instances of F (e.g., 1, 2, 3 instances of F) are independently substituted by F⁵.

In certain embodiments, each instance of F is substituted by an amino acid selected from F¹, F², F³, F⁴, and F⁵. In certain embodiments, each instance of F is substituted by F¹. In certain embodiments, each instance of F is substituted by F². In certain embodiments, each instance of F is substituted by F³. In certain embodiments, each instance of F is substituted by F⁴. In certain embodiments, each instance of F is substituted by F⁵.

Also provided herein are peptides and pharmaceutically acceptable salts thereof comprising any amino acid sequence provided herein (e.g., SEQ ID NOs: 1-32, 65-70, 74-78), optionally wherein one or more instances of K are independently substituted by an amino acid selected from Orn, Dab, Dap, R, and hArg; and further optionally wherein one or more instances of F are independently substituted by a modified phenylalanine (e.g., F¹, F², F³, F⁴, and F⁵).

In certain embodiments, an amino acid sequence provided herein (e.g., SEQ ID NOs: 1-32, 65-70, 74-78) comprises 1 to 6 (e.g., 1, 2, 3, 4, 5, 6) amino acid substitutions, inclusive; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) comprises 1 to 5 (e.g., 1, 2, 3, 4, 5) amino acid substitutions, inclusive; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) comprises 1 to 4 (e.g., 1, 2, 3, 4) amino acid substitutions, inclusive; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) comprises 1 to 3 (e.g., 1, 2, 3) amino acid substitutions, inclusive; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) comprises 1 or 2 amino acid substitutions; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) comprises 1 amino acid substitution; optionally further wherein one or more instances of K (e.g., each instance of K) are independently substituted by an amino acid selected from Orn, Dab, Dap, R, and hArg; and optionally further wherein one or more instances of F (e.g., each instance of F) are independently substituted by a modified phenylalanine (e.g., F¹, F⁵, F⁴, F², or F³).

In certain embodiments, an amino acid sequence provided herein (e.g., SEQ ID NOs: 1-32, 65-70, 74-78) can comprise any combination of the following amino acid substitutions:
(i) 1 to 6 (e.g., 1, 2, 3, 4, 5, 6) amino acid substitutions, inclusive (e.g., G8A and/or G18A);
(ii) one or more instances of K (e.g., each instance of K) are independently substituted by an amino acid selected from Orn, Dab, Dap, R, and hArg; and/or
(iii) one or more instances of F (e.g., each instance of F) are independently substituted by a modified phenylalanine (e.g., F¹, F⁵, F⁴, F², and F³).

### Peptide C-Terminus Modifications

Peptides provided herein can comprise one or more additional modifications anywhere on the peptide (e.g., on an amino acid sidechain, on an α-carbon of an amino acid, on a peptidic nitrogen, at the C-terminus, N-terminus, etc.). Peptides provided herein can comprise modifications to the C-terminus and/or N-terminus of the polypeptide. In certain embodiments, a polypeptide comprises a modified C-terminus. Examples of C-terminus modifications are described herein.

In certain embodiments, a peptide provided herein comprises an amidated C-terminus. Traditionally, peptides comprise a carboxyl group (—C(═O)OH) at the C-terminus. Peptides provided herein may comprise an amide at the C-terminus (e.g., —C(═O)NR₂, wherein the group NR₂ is NH₂, monosubstituted amino, disubstituted amino, or trisubstituted amino), referred to as “amidated C-terminus.” For example, a peptide with a “C-terminus amidated with —NH₂” comprises the group —C(═O)NH₂ at the C-terminus instead of carboxyl (—C(═O)OH). An amidated C-terminus can also be represented by including —NR₂ (e.g., —NH₂) at the end of an amino acid sequence.

In certain embodiments, a peptide provided herein comprises a hydroxamidated C-terminus (i.e., comprises the group —C(═O)NHOH at the C-terminus instead of carboxyl (—C(═O)OH)). A hydroxamidated C-terminus can also be represented by including —NHOH at the end of an amino acid sequence.

Peptides provided herein may also be amidated at the C-terminus with an amino acid, peptide, or protein. The amino acid, peptide, or protein can be natural or unnatural. In certain embodiments, the peptide comprises a peptide conjugated to the C-terminus. In certain embodiments, the peptide is from 2 to 6 amino acids in length, inclusive, and comprises amino acids selected from G, E, S, A, and K.

In certain embodiments, the peptide is from 2 to 6 amino acids in length, inclusive, and comprises amino acids selected from G, E, and S. In certain embodiments, the peptide is from 2 to 6 amino acids in length, inclusive, and comprises amino acids selected from G and E. In certain embodiments, the peptide is 2 amino acids in length and comprises amino acids selected from G and E. In certain embodiments, the peptide is 3 amino acids in length and comprises amino acids selected from G and E. In certain embodiments, the peptide is 4 amino acids in length and comprises amino acids selected from G and E.

Non-limited examples of peptides which can be conjugated to the C-terminus are the following:

GE,
AG,
AA,
AK,
GG,
GGE,
GGS,
GGG,
GGK,
GGQ,
(SEQ ID NO: 88)
GGGE,
(SEQ ID NO: 89)
GGEE,
or
(SEQ ID NO: 90)
GGSGGS.

Peptides provided herein may also comprise a small molecule, lipophilic group, or polymer conjugated to the C-terminus of the peptide.

In certain embodiments, the peptide comprises a lipophilic group conjugated to the C-terminus of the peptide. In certain embodiments, the lipophilic group is a lipid or fatty acid. In certain embodiments, the lipophilic group is a hydrocarbon chain.

In certain embodiments, the peptide comprises a polymer conjugated to the C-terminus of the peptide. In certain embodiments, the polymer is a polyether, e.g., polyethylene glycol (PEG). In certain embodiments, the polymer is PEG.

In certain embodiments, the peptide is amidated at the C-terminus with a group of the following formula: —NH—(PEG)-CONH₂, wherein PEG is polyethylene glycol. In certain embodiments, the peptide is amidated at the C-terminus with a group of the following formula: —NH(CH₂CH₂O)₁₋₂₀CH₂CH₂CONH₂. In certain embodiments, the polymer is amidated at the C-terminus with a group of one of the following formulae: —NHCH₂CH₂OCH₂CH₂CONH₂, —NH(CH₂CH₂O)₂—CH₂CH₂CONH₂, —NH(CH₂CH₂O)₃—CH₂CH₂CONH₂, —NH(CH₂CH₂O)₄—CH₂CH₂CONH₂, or —NH(CH₂CH₂O)₅—CH₂CH₂CONH₂.

In certain embodiments, the peptide comprises a small molecule conjugated to the C-terminus of the peptide. In certain embodiments, the small molecule is an antimicrobial agent such as a small-molecule antibiotic. In certain embodiments, the small molecule is cyclic antimicrobial peptide conjugated to the C-terminus. Polymyxins are a non-limiting example of cyclic antimicrobial peptides.

### Unstapled Peptides

Also provided herein are unstapled (i.e., uncrosslinked) peptides which can serve as synthetic precursors to the stapled (i.e., crosslinked) peptides provided herein. Such unstapled peptides may be referred to an “unstapled precursor peptides,” “unstapled precursors,” and the like. Unstapled precursor peptides comprise pairs of amino acids which comprise reactive moieties capable of reacting to form crosslinks.

Provided herein are unstapled (i.e., uncrosslinked) peptides comprising 10 or more contiguous amino acids of Esculentin-1A (SEQ ID NO: 73); wherein:
exactly two amino acids of the 10 or more contiguous amino acids of Esculentin-1A are independently substituted by X¹ and X²; and
X¹ and X² are each independently amino acids comprising reactive moieties capable of forming a crosslink with the other;
optionally wherein the amino acid sequence includes 1 to 3 amino acid substitutions, inclusive, at positions other than X¹ and X². In certain embodiments, X¹ and X² are independently thiol-containing amino acids. In certain embodiments, X¹ and X² are cysteine (C). In certain embodiments, X¹ and X² are homocysteine.

In certain embodiments, the unstapled peptide or pharmaceutically acceptable salt thereof comprises 20 or more contiguous amino acids of Esculentin-1A (SEQ ID NO: 73); optionally wherein the amino acid sequence includes 1 to 6 amino acid substitutions, inclusive, at positions other than X¹ and X². In certain embodiments, X¹ and X² are independently thiol-containing amino acids. In certain embodiments, X¹ and X² are cysteine (C). In certain embodiments, X¹ and X² are homocysteine.

As described herein, in certain embodiments, X¹ and X² are separated by 3 amino acids. In certain embodiments, X¹ and X² are separated by 4 amino acids. In certain embodiments, X¹ and X² are separated by 5 amino acids. In certain embodiments, X¹ and X² are separated by 6 amino acids.

Provided herein are unstapled (i.e., uncrosslinked) peptides, and pharmaceutically acceptable salt thereof, comprising 10 or more contiguous amino acids of Esculentin-1A (SEQ ID NO: 73), wherein:
2 or 4 amino acids of the 10 or more contiguous amino acids of Esculentin are substituted by cysteine (C);
optionally wherein the amino acid sequence includes 1 to 3 additional amino acid substitutions, inclusive.

In certain embodiments, the unstapled (i.e., uncrosslinked) peptide or pharmaceutically acceptable salt thereof comprises 20 or more contiguous amino acids of Esculentin-1A (SEQ ID NO: 73); wherein:
2 or 4 amino acids of the 20 or more contiguous amino acids of Esculentin are substituted by cysteine (C);
optionally wherein the amino acid sequence includes 1 to 6 additional amino acid substitutions, inclusive.

In certain embodiments, at least two C are separated by 3 amino acids. In certain embodiments, at least two C are separated by 4 amino acids. In certain embodiments, at least two C are separated by 5 amino acids. In certain embodiments, at least two C are separated by 6 amino acids.

In certain embodiments, the 20 or more contiguous amino acids of Esculentin-1A are SEQ ID NO: 75. In certain embodiments, SEQ ID NO: 75 comprises a N21G amino acid substitution (e.g., SEQ ID NO: 76).

In certain embodiments, an unstapled peptide or pharmaceutically acceptable salt thereof provided herein comprises one of SEQ ID NOs: 1-32, 65-70, 74-78. In certain embodiments, an unstapled peptide provided herein is of one of SEQ ID NOs: 1-32, 65-70, 74-78, or a pharmaceutically acceptable salt thereof. In certain embodiments, an unstapled peptide provided herein is of one of SEQ ID NOs: 1-32, 65-70, 74-78, or a pharmaceutically acceptable salt thereof, wherein the C-terminus of the peptide is amidated with —NH₂.

In certain embodiments, an unstapled (i.e., uncrosslinked) peptide provided herein comprises one of the following amino acid sequences:

(SEQ ID NO: 33)
C I F S C L A G K K I K N L L I S G L K G,
(SEQ ID NO: 34)
G C F S K C A G K K I K N L L I S G L K G,
(SEQ ID NO: 35)
G I C S K L C G K K I K N L L I S G L K G,
(SEQ ID NO: 36)
G I F C K L A C K K I K N L L I S G L K G,
(SEQ ID NO: 37)
G I F S C L A G C K I K N L L I S G L K G,
(SEQ ID NO: 38)
G I F S K C A G K C I K N L L I S G L K G,
(SEQ ID NO: 39)
G I F S K L C G K K C K N L L I S G L K G,
(SEQ ID NO: 82)
G I F S K L C G K K C K N L L I S G L K G G G,
(SEQ ID NO: 40)
G I F S K L A C K K I C N L L I S G L K G,
(SEQ ID NO: 41)
G I F S K L A G C K I K C L L I S G L K G,
(SEQ ID NO: 42)
G I F S K L A G K C I K N C L I S G L K G,
(SEQ ID NO: 43)
G I F S K L A G K K C K N L C I S G L K G,
(SEQ ID NO: 44)
G I F S K L A G K K I C N L L C S G L K G,
(SEQ ID NO: 45)
G I F S K L A G K K I K C L L I C G L K G,
(SEQ ID NO: 46)
G I F S K L A G K K I K N C L I S C L K G,
(SEQ ID NO: 47)
G I F S K L A G K K I K N L C I S G C K G,
(SEQ ID NO: 48)
G I F S K L A G K K I K N L L C S G L C G,
(SEQ ID NO: 49)
G I F S K L A G K K I K N L L I C G L K C,

or a pharmaceutically acceptable salt thereof.

In certain embodiments, an unstapled (i.e., uncrosslinked) peptide provided herein comprises one of the following amino acid sequences:

(SEQ ID NO: 79)
G I F S K L C G K K C K N L L I S G L K G G G G,
(SEQ ID NO: 80)
G I F S K L C G K K C K N L L I S G L K G G G K,
(SEQ ID NO: 81)
G I F S K L C G K K C K N L L I S G L K G A G,
(SEQ ID NO: 83)
G I F S K L C G K K C K N L L I S G L K G A K,
(SEQ ID NO: 84)
G I F S K L C A K K C K N L L I S G L K G,
(SEQ ID NO: 85)
G I F S K L C G K K C K N L L I S A L K G,
(SEQ ID NO: 86)
G I F S K L C A K K C K N L L I S A L K G,

or a pharmaceutically acceptable salt thereof.

In certain embodiments, an unstapled peptide or pharmaceutically acceptable salt thereof is of any one of SEQ ID NOs: 33-49 and 82. In certain embodiments, an unstapled peptide or pharmaceutically acceptable salt thereof is of any one of SEQ ID NOs: 33-49; wherein the C-terminus is amidated with —NH₂. In certain embodiments, an unstapled peptide or pharmaceutically acceptable salt thereof is of any one of SEQ ID NO: 82; wherein the wherein the C-terminus is —CO₂H. In certain embodiments, an unstapled peptide or pharmaceutically acceptable salt thereof is of any one of SEQ ID NOs: 79-81 and 83-86. In certain embodiments, an unstapled peptide or pharmaceutically acceptable salt thereof is of any one of SEQ ID NOs: 79-81 and 83-86; wherein the C-terminus is amidated with —NH₂. In certain embodiments, an unstapled peptide or pharmaceutically acceptable salt thereof is of any one of SEQ ID NO: 79-81 and 83-86; wherein the wherein the C-terminus is —CO₂H.

In certain embodiments an unstapled (i.e., uncrosslinked) peptide may comprise the amino acid sequence: JIFSKCAGKCIKNLLISGLKG (SEQ ID NO: 87).

In certain embodiments, an unstapled (i.e., uncrosslinked) peptide provided herein comprises one of the following amino acid sequences:

(SEQ ID NO: 50)
C I F S K L A C K K I K N L L I S G L K G,
(SEQ ID NO: 51)
G C F S K L A G C K I K N L L I S G L K G,
(SEQ ID NO: 52)
G I C S K L A G K C I K N L L I S G L K G,
(SEQ ID NO: 53)
G I F C K L A G K K C K N L L I S G L K G,
(SEQ ID NO: 54)
G I F S C L A G K K I C N L L I S G L K G,
(SEQ ID NO: 55)
G I F S K C A G K K I K C L L I S G L K G,
(SEQ ID NO: 56)
G I F S K L A C K K I K N L C I S G L K G,
(SEQ ID NO: 57)
G I F S K L A G C K I K N L L C S G L K G,
(SEQ ID NO: 58)
G I F S K L A G K C I K N L L I C G L K G,
(SEQ ID NO: 59)
G I F S K L A G K K C K N L L I S C L K G,
(SEQ ID NO: 60)
G I F S K L A G K K I C N L L I S G C K G,
(SEQ ID NO: 61)
G I F S K L A G K K I K C L L I S G L C G,
(SEQ ID NO: 62)
G I F S K L A G K K I K N C L I S G L K C,
(SEQ ID NO: 63)
G I F S K L C G K K I K N C L I S G L K G,

or a pharmaceutically acceptable salt thereof.

In certain embodiments, an unstapled peptide or pharmaceutically acceptable salt thereof is of any one of SEQ ID NOs: 50-63. In certain embodiments, the unstapled peptide or pharmaceutically acceptable salt thereof is of any one of SEQ ID NOs: 50-63; wherein the wherein the C-terminus is amidated with —NH₂.

In certain embodiments, an unstapled (i.e., uncrosslinked) peptide provided herein comprises the following amino acid sequence:

(SEQ ID NO: 64)
G I F S K L C G K K C K N L L I C G L K C,

or a pharmaceutically acceptable salt thereof.

In certain embodiments, an unstapled peptide or pharmaceutically acceptable salt thereof is of SEQ ID NO: 64. In certain embodiments, the unstapled peptide or pharmaceutically acceptable salt thereof is of SEQ ID NO: 64; wherein the wherein the C-terminus is amidated with —NH₂.

As the unstapled peptides provided herein can serve as synthetic precursors to the stapled peptides provided herein, the various numbers, positions, and types of amino acid substitutions described herein with respect to the stapled peptides (e.g., supra) also apply to the unstapled peptides provided herein. Additionally, the possible C-terminus modifications described herein with respect to the stapled peptides (e.g., supra) apply to the unstapled peptides provided herein.

### Reactive Moieties

As described herein, the unstapled (i.e., uncrosslinked) peptides comprise pairs of amino acids (e.g., X¹ and X²; X³ and X⁴) which comprise reactive moieties capable of forming crosslinks. In preferred embodiments, the pairs of amino acids (e.g., X¹ and X²; X³ and X⁴) comprise α-sidechains comprising the reactive moieties.

For the purposes of this disclosure, “reactive moieties” are any chemical moieties capable of reacting with another chemical moiety to form a covalent bond or covalent bonds. Non-limiting examples of reactive moieties include alkenes, alkynes, alcohols, amines, thiols, azides, esters, amides, halogens, and the like. In certain embodiments, two reactive moieties are capable of reacting directly with each other to form a crosslink (e.g., alkenes undergoing ring-closing metathesis (RCM), or an alkyne and an azide undergoing 1,3-dipolar cycloaddition to form a triazole). In other embodiments, two reactive moieties react with an intervening crosslinking reagent to form a crosslink (e.g., two cysteine residues reacting with a dihalide (e.g., dibromo xylene)). In certain embodiments, the reactive moieties are click chemistry moieties. “Click chemistry” moieties are any moieties that can be used in click chemistry reactions (infra).

As described herein, in certain embodiments, the reactive moieties are thiols (e.g., capable of reacting with a crosslinking reagent such as a dihalide (e.g., α,α′-dibromo xylene) to form a dithio crosslink). In certain embodiments, X¹, X², X³, and X⁴ independently comprise α-sidechains of the following formula:

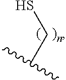

wherein each n is independently an integer from 1-10, inclusive. In certain embodiments, at least one instance of n is 1. In certain embodiments, each instance of n is 1.

In certain embodiments, each of X¹, X², X³, and X⁴ are cysteine (C). In certain embodiments, each of X¹, X², X³, and X⁴ are homocysteine. In certain embodiments, X¹ and X² are C. In certain embodiments, X³ and X⁴ are C. In certain embodiments, X¹ and X² are homocysteine. In certain embodiments, X³ and X⁴ are homocysteine.

In certain embodiments, the reactive moieties are independently selected from alkenes and alkynes (e.g., capable of undergoing RCM reactions to form a hydrocarbon crosslink). In certain embodiments, the reactive moieties are terminal alkenes. In certain embodiments, the reactive moieties are terminal alkynes.

In certain embodiments, X¹, X², X³, and X⁴ independently comprise α-sidechains of the following formula:

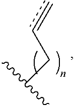

wherein each n is independently an integer from 1-10, inclusive. In certain embodiments, at least one instance of n is 3. In certain embodiments, the sum of two n between crosslinking amino acids is 6.

In certain embodiments, X¹, X², X³, and X⁴ are independently α,α-disubstituted amino acids. In certain embodiments, X¹, X², X³, and X⁴ each independently comprise the formula:

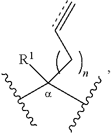

wherein α denotes the α-carbon of the amino acids; and each instance of R¹ is optionally substituted C₁₋₆ alkyl. In certain embodiments, R¹ is methyl.

In certain embodiments, at least one instance of X¹, X², X³, and X⁴ is an amino acid of the following formula:

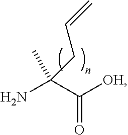

wherein each n is independently an integer from 1-10, inclusive.

In certain embodiments, at least one instance of X¹, X², X³, and X⁴ is the amino acid S⁵:

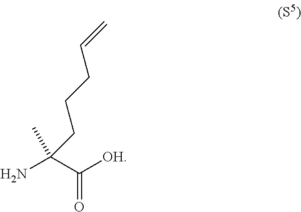

In certain embodiments, X¹ is S⁵. In certain embodiments, X² is S⁵. In certain embodiments, X³ is S⁵. In certain embodiments, X⁴ is S⁵. In certain embodiments, X¹, X², X³, and X⁴ are S⁵.

In certain embodiments, X¹ is R⁸. In certain embodiments, X² is R⁸. In certain embodiments, X³ is R⁸. In certain embodiments, X⁴ is R⁸. In certain embodiments, X¹, X², X³, and X⁴ are R⁸.

In certain embodiments, the reactive moieties are azides and alkynes (e.g., capable of reacting via 1,3-cycloaddition to form a triazolylene crosslink). In certain embodiments, one of X¹ and X² comprises an alkyne reactive moiety, and the other comprises an azide reactive moiety. In certain embodiments, one of X³ and X⁴ comprises an alkyne reactive moiety, and the other comprises an azide reactive moiety.

In certain embodiments, one of X¹ and X² comprises an α-sidechain of the following formula:

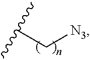

and the other comprises an α-sidechain of the following formula:

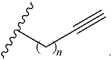

In certain embodiments, one of X³ and X⁴ comprises an α-sidechain of the following formula:

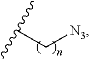

and the other comprises an α-sidechain of the following formula:

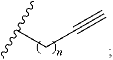

wherein each n is independently an integer from 1-10, inclusive. In certain embodiments, one of X¹ and X² comprises an α-sidechain of the following formula:

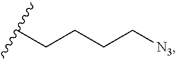 [SMILES: *CCCCN=[N+]=[N-]]

and the other comprises an α-sidechain of the following formula:

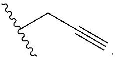 [SMILES: *CC#C]

In certain embodiments, one of X³ and X⁴ comprises an α-sidechain of the following formula:

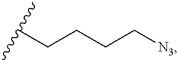 [SMILES: *CCCCN=[N+]=[N-]]

and the other comprises an α-sidechain of the following formula:

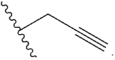 [SMILES: *CC#C]

In certain embodiments, one of X¹ and X² is J, and the other is Azi. In certain embodiments, one of X³ and X⁴ is J, and the other is Azi.

### Methods of Preparing Stapled Peptides

Also provided herein are methods of preparing crosslinked (i.e., stapled) peptides described herein comprising reacting uncrosslinked (i.e., unstapled) peptides under conditions sufficient to form the crosslinks.

For example, in the case of reactions between thiol reactive moieties to form dithio crosslinks, thiol-stapling reactions may be used. “Thiol-stapling” involves reacting two thiol moieties in the presence of a crosslinking reagent (e.g., a dihalide) to form the crosslink. Examples of thiol-stapling reactions can be found in, e.g., US Patent Application Publication No. US 2019/0382443 A1, published Dec. 19, 2019; U.S. Pat. No. 9,670,484 B2, issued Jun. 6, 2017; U.S. Pat. No. 9,644,201 B2, issued May 9, 2017; US Patent Application Publication No. 2017/0067045 A1, published Mar. 9, 2017; Peraro, L. et al. Methods in Enzymology vol. 580 (2016): 303-32; and Kale, S. S. et al. Nature Chem 10, 715-723 (2018), the entire contents of each of which is incorporated herein by reference.

For example, provided below in Table 4 are examples of crosslinking reagents and the dithio crosslinks they are capable of forming via thiol-stapling reactions with cysteine residues:

TABLE 4
Thiol-Stapling Crosslinking Reagents

| Crosslinking Reagent | Dithio Crosslink |
|---|---|
| 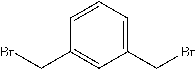 | (mxy) |
| 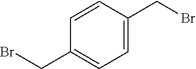 | (pxy) |
| 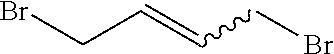 | (but) |
| 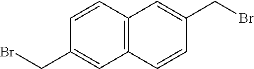 [SMILES: BrCc1ccc2cc(CBr)ccc2c1] | (bbn) |
| 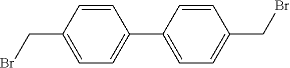 [SMILES: BrCc1ccc(-c2ccc(CBr)cc2)cc1] | (bbf) |
| 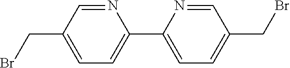 [SMILES: BrCc1ccc(-c2ccc(CBr)cn2)nc1] | (bbp) |
| 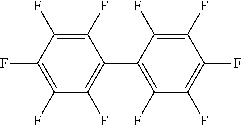 [SMILES: Fc1c(F)c(F)c(-c2c(F)c(F)c(F)c(F)c2F)c(F)c1F] | (pfb) |
| 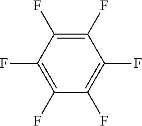 [SMILES: Fc1c(F)c(F)c(F)c(F)c1F] | (hfb) |

A general scheme of a thiol-stapling reaction is shown below in Scheme 1, wherein LG is a leaving group (e.g., halogen, e.g., bromine), and L² is as described herein.

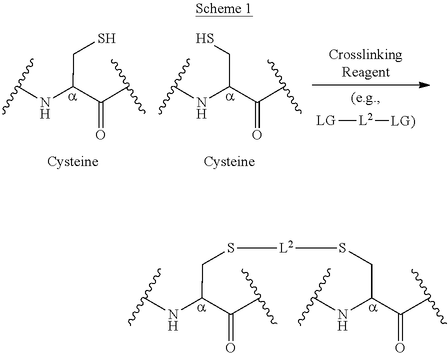

In the case of reactions between alkene or alkyne reactive moieties to form hydrocarbon crosslinks, ring-closing metathesis (RCM) reactions may be used. In certain embodiments, the RCM reaction involves reacting the peptide in the presence of a ruthenium complex. In certain embodiments, the ruthenium complex is, e.g., a Grubbs, Grubbs II, Hoveyda-Grubbs I, or Hoveyda-Grubbs II catalyst.

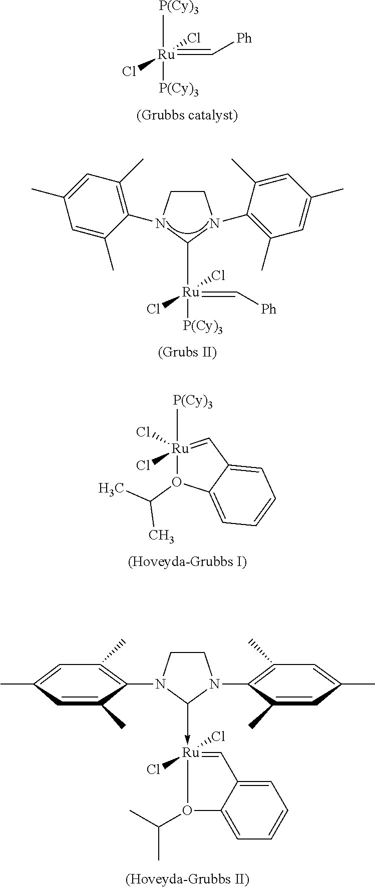

Other examples of metathesis (e.g., RCM) catalysts, reagents, and reaction conditions useful in the present methods can be found in, e.g., Schrodi, Y.; Pederson, R. L. Aldrichimica Acta 2007, 40, 45; Adv. Synth. Catal. 2007, 349, 1-268; Grubbs, R. H. Tetrahedron 2004, 60, 7117; Handbook of Metathesis; Grubbs, R. H., Ed.; Wiley-VCH: Weinheim, 2003; Vols. 1-3; Trnka, T. M.; Grubbs, R. H. Acc. Chem. Res. 2001, 34, 18; Furstner, A. Angew. Chem., Int. Ed. 2000, 39, 3012; Schuster, M.; Blechert, S. Angew. Chem., Int. Ed. 1997, 36, 2036; Ritter, T. et al. Organometallics 2006, 25, 5740; Chatterjee, A. K. et al. J. Am. Chem. Soc. 2000, 122, 3783; Chatterjee, A. K.; Grubbs, R. H. Org. Lett. 1999, 1, 1751; Murelli, R. P.; Snapper, M. L. Org. Lett. 2007, 9, 1749; Stewart, I. C. et al. Org. Lett. 2007, 9, 1589; Ung, T. et al. Organometallics 2004, 23, 5399; Benitez, D.; Goddard, W. A., III. J. Am. Chem. Soc. 2005, 127, 12218; Love, J. A. et al. Angew. Chem., Int. Ed. 2002, 41, 4035; Sanford, M. S. et al. Organometallics 2001, 20, 5314; Choi, T. L.; Grubbs, R. H. Angew. Chem. 2003, 115, 1785; Ritter, T. et al. Organometallics 2006, 25, 5740, the entire contents of each of which is incorporated herein by reference.

As another example, in the case of reactions between an azide and alkyne reactive moieties to form triazolylene crosslinks, alkyne-azide 1,3-cycloadditions may be used (e.g., the Huisgen alkyne-azide cycloaddition). In certain embodiments, the alkyne-azide cycloaddition is copper-catalyzed. In certain embodiments, the alkyne-azide cycloaddition is strain-promoted. Examples of alkyne-azide reactions can be found in, e.g., Kolb, Finn and Sharpless Angewandte Chemie International Edition (2001) 40: 2004-2021; Kolb and Sharpless, Drug Discov Today (2003) 24: 1128-1137; and Evans, Australian Journal of Chemistry (2007) 60: 384-395.

Other click chemistry reactions may be used to form the crosslinks of the peptides described herein. “Click chemistry” is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395. Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition).

### Pharmaceutical Compositions, Kits, and Administration

The present disclosure provides pharmaceutical compositions comprising a peptide disclosed herein, or a pharmaceutically acceptable salt thereof. The pharmaceutical composition may comprise one or more pharmaceutically acceptable carriers/excipients. In certain embodiments, a peptide described herein is provided in an effective amount in the pharmaceutical composition. In certain embodiments, the effective amount is a therapeutically effective amount (e.g., for treating a bacterial infection in a subject). In certain embodiments, the effective amount is a prophylactically effective amount (e.g., for preventing a bacterial infection in a subject). In certain embodiments, the effective amount is an amount effective for killing and/or inhibiting the growth of bacteria in a subject or biological sample.

Pharmaceutical compositions described herein can be prepared by any method known in the art of pharmacology. In general, such preparatory methods include bringing the peptide described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol*), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof. In certain embodiments, the formulation comprises a polymer excipient. In certain embodiments, the formulation comprises a polyether. In certain embodiments, the formulation comprises polyethylene glycol (PEG) (e.g., PEG200, PEG300, PEG400, and the like).

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a peptide described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the peptide in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

A pharmaceutical composition described herein can be prepared, packaged, and/or sold in a formulation suitable for pulmonary administration via the buccal cavity. Such a formulation may comprise dry particles which comprise the active ingredient. Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device.

Formulations described herein as being useful for pulmonary delivery are useful for intranasal delivery of a pharmaceutical composition described herein. Formulations for nasal administration may, for example, comprise from about as little as 0.1% (w/w) to as much as 100% (w/w) of the active ingredient, and may comprise one or more of the additional ingredients described herein.

A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Peptides provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The peptides and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, topical administration, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a peptide required to achieve an effective amount will vary from subject to subject, depending, for example, on species, age, and general condition of a subject, severity of the side effects or disorder, identity of the particular peptide, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a peptide described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a peptide described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A peptide or composition, as described herein, can be administered in combination with one or more additional pharmaceutical agents (e.g., therapeutically and/or prophylactically active agents). The peptides or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a peptide described herein and an additional pharmaceutical agent shows a synergistic effect that is absent in a pharmaceutical composition including one of the peptide and the additional pharmaceutical agent, but not both. In some embodiments, the additional pharmaceutical agent achieves a desired effect for the same disorder. In some embodiments, the additional pharmaceutical agent achieves different effects.

The peptide or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic peptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

The additional pharmaceutical agents include, but are not limited to, anti-proliferative agents, anti-cancer agents, anti-angiogenesis agents, steroidal or non-steroidal anti-inflammatory agents (NSAIDs), immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins.

In certain embodiments, the additional pharmaceutical agent is an antimicrobial agent. In certain embodiments, the additional pharmaceutical agent is an antibiotic. In certain embodiments, the additional pharmaceutical agent is selected from the group consisting of polymyxins, rifampicin, ofloxacin, vancomycin, and meropenem.

Each additional pharmaceutical agent may be administered at a dose and/or on a time schedule determined for that pharmaceutical agent. The additional pharmaceutical agents may also be administered together with each other and/or with the peptide or composition described herein in a single dose or composition or administered separately in different doses or compositions. The particular combination to employ in a regimen will take into account compatibility of the peptide described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). The kits provided may comprise a pharmaceutical composition or peptide described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or peptide described herein. In some embodiments, the pharmaceutical composition or peptide described herein provided in the first container and the second container are combined to form one unit dosage form. Thus, in one aspect, provided are kits including a first container comprising a peptide or pharmaceutical composition described herein. In certain embodiments, the kits are useful for treating a disease (e.g., bacterial infection) in a subject in need thereof. In certain embodiments, the kits are useful for preventing a disease (e.g., bacterial infection) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits provide instructions for treating a disease (e.g., bacterial infection) in a subject in need thereof. In certain embodiments, the kits provide instructions for preventing a disease (e.g., bacterial infection) in a subject in need thereof. A kit described herein may include one or more additional pharmaceutical agents described herein as a separate composition.

### Lipid Formulations

Also provided herein are formulations (i.e., pharmaceutical compositions) comprising one or more stapled antimicrobial peptides (StAMPs) and one or more lipids (“lipid formulations”). The lipid formulation may be a liposomal, micellar, lipid nanoparticle (“LNP”) formulation, or the like. In certain embodiments, the lipid formulation is a micellar formulation. In certain embodiments, the lipid formulation is a liposomal formulation. In certain embodiments, the lipid formulation is an LNP formulation. In certain embodiments, a StAMP is encapsulated in a micelle, liposome, LNP, or the like.

In certain embodiments, the lipid formulation comprises a peptide (e.g., StAMP) provided herein. In certain embodiments, the peptide (e.g., StAMP) is a peptide described in, e.g., International PCT application Publication Nos. WO 2017/004591, published Jan. 5, 2017; and WO 2019/018499, published Jan. 24, 2019, the entire contents of each of which are incorporated herein by reference.

Lipid formulations provided herein comprise one or more lipids. In certain embodiments, the lipid comprises a phospholipid (e.g., 1,2-distearoyl-sn-glycero-3-phosphoethanolamine (DSPE)). In certain embodiments, the lipid comprises polyethylene glycol (PEG). In certain embodiments, the lipid is a PEGylated phospholipid. A “PEGylated phospholipid” is a lipid comprising a phospholipid conjugated to PEG. For example, in certain embodiments, the PEGylated phospholipid comprises DSPE and PEG (i.e., “DSPE-PEG”). In certain embodiments, the PEGylated phospholipid is DSPE-MPEG (e.g., DSPE-MPEG(2000)). Lipids (e.g., phospholipids) include pharmaceutically acceptable salts of the lipids. For example, the structure of DSPE-MPEG is shown below:

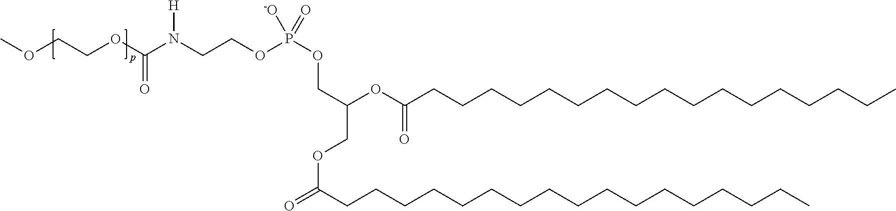

The lipid formulation can comprise any ratio of peptide:lipid. In certain embodiments, the lipid formulation comprises a peptide:lipid ratio from about 1:1 to about 1:25 (w/w), inclusive. In certain embodiments, the lipid formulation comprises a peptide:lipid ratio from about 1:2 to about 1:25 (w/w), inclusive. In certain embodiments, the lipid formulation comprises a peptide:lipid ratio from about 1:2.5 to about 1:20 (w/w), inclusive. In certain embodiments, the lipid formulation comprises a peptide:lipid ratio from about 1:2 to about 1:3 (w/w), inclusive. In certain embodiments, the lipid formulation comprises a peptide:lipid ratio from about 1:4 to about 1:6 (w/w), inclusive. In certain embodiments, the lipid formulation comprises a peptide:lipid ratio from about 1:8 to about 1:12 (w/w), inclusive. In certain embodiments, the lipid formulation comprises a peptide:lipid ratio from about 1:15 to about 1:25 (w/w), inclusive.

In certain embodiments, the lipid formulation comprises a peptide:lipid ratio of about 1:2.5 (w/w). In certain embodiments, the lipid formulation comprises a peptide:lipid ratio of about 1:5 (w/w). In certain embodiments, the lipid formulation comprises a peptide:lipid ratio of about 1:10 (w/w). In certain embodiments, the lipid formulation comprises a peptide:lipid ratio of about 1:20 (w/w).

For example, in certain embodiments, the lipid formulation comprises a peptide:DSPE-MPEG ratio from about 1:1 to about 1:25 (w/w), inclusive. In certain embodiments, the lipid formulation comprises a peptide:DSPE-MPEG ratio from about 1:2 to about 1:25 (w/w), inclusive. In certain embodiments, the lipid formulation comprises a peptide:DSPE-MPEG ratio from about 1:2.5 to about 1:20 (w/w), inclusive. In certain embodiments, the lipid formulation comprises a peptide:DSPE-MPEG ratio from about 1:2 to about 1:3 (w/w), inclusive. In certain embodiments, the lipid formulation comprises a peptide:DSPE-MPEG ratio from about 1:4 to about 1:6 (w/w), inclusive. In certain embodiments, the lipid formulation comprises a peptide:DSPE-MPEG ratio from about 1:8 to about 1:12 (w/w), inclusive. In certain embodiments, the lipid formulation comprises a peptide:DSPE-MPEG ratio from about 1:15 to about 1:25 (w/w), inclusive.

In certain embodiments, the lipid formulation comprises a peptide:DSPE-MPEG ratio of about 1:2.5 (w/w). In certain embodiments, the lipid formulation comprises a peptide:DSPE-MPEG ratio of about 1:5 (w/w). In certain embodiments, the lipid formulation comprises a peptide:DSPE-MPEG ratio of about 1:10 (w/w). In certain embodiments, the lipid formulation comprises a peptide:DSPE-MPEG ratio of about 1:20 (w/w).

### Methods of Treatment and Uses

Peptides (e.g., stapled peptides, e.g., StAMPs) of the present disclosure have antimicrobial activity and are therefore useful in various methods such as treating infectious diseases (e.g., bacterial infections) in a subject, and killing and/or inhibiting the growth of microbes (e.g., bacteria).

Provided herein are methods of treating and/or preventing an infectious disease in a subject comprising administering to the subject a peptide (e.g., StAMP) described herein, or a pharmaceutically acceptable salt thereof, or a pharmaceutical composition thereof. In certain embodiments, the infectious disease is a bacterial infection. In certain embodiments, the infectious disease is a viral infection. In certain embodiments, the infectious disease is a protozoal infection. In certain embodiments, the infectious disease is a fungal infection. In certain embodiments, the infectious disease is a parasitic infection.

In certain embodiments, the method is for treating an infectious disease. In certain embodiments, the method is for preventing an infectious disease.

Provided herein are methods of treating and/or preventing a bacterial infection in a subject comprising administering to the subject a peptide (e.g., StAMP) described herein, or a pharmaceutically acceptable salt thereof, or a pharmaceutical composition thereof. In certain embodiments, the bacterial infection is a Gram-positive bacterial infection, i.e., is caused by Gram-positive bacteria. In certain embodiments, the bacterial infection is a Gram-negative bacterial infection, i.e., is caused by Gram-negative bacteria.

In certain embodiments, the method is for treating a bacterial infection. In certain embodiments, the method is for preventing a bacterial infection.

The Gram-negative bacterial infection may be caused by any Gram-negative bacteria described herein. In certain embodiments, the bacterial infectious is caused by Acinetobacter, Escherichia, Pseudomonas, Neisseria, Chlamydia, Yersinia, Proteus, Enterobacter, Serratia, Helicobacter, Salmonella, Shigella, Moraxella, Stenotrophomonas, Bdellovibrio, Klebsiella, Legionella, or acetic acid bacteria. In certain embodiments, the bacterial infection is caused by Acinetobacter baumannii, Escherichia coli, Pseudomonas aeruginosa, Neisseria gonorrhoeae, Chlamydia trachomatis, Yersinia pestis, Proteus mirabilis, Enterobacter cloacae, Serratia marcescens, Helicobacter pylori, Salmonella enteritidis, Salmonella typhi, Shigella, Klebsiella pneumoniae, or Legionella pneumophila bacteria, or a Gram-negative bacteria that contains a MCR-1 gene.

In certain embodiments, the bacterial infection is caused by an Escherichia, Acinetobacter, Pseudomonas, or Klebsiella species. In certain embodiments, the bacterial infection is caused by Escherichia coli (E. coli), Acinetobacter baumannii (A. baumannii), Pseudomonas aeruginosa (P. aeruginosa), or Klebsiella pneumoniae (K. pneumoniae). In certain embodiments, the bacterial infection is caused by a Stenotrophomonas, Burkholderia, or Klebsiella species. In certain embodiments, the bacterial infection is caused by Stenotrophomonas maltophilia (S. maltophilia), Burkholderia cepacia (B. cepacia), or Klebsiella oxytoca (K. oxytoca). In certain embodiments, the bacterial infection is caused by E. coli. In certain embodiments, the bacterial infection is caused by A. baumannii. In certain embodiments, the bacterial infection is caused by P. aeruginosa. In certain embodiments, the bacterial infection is caused by K. pneumoniae. In certain embodiments, the bacterial infection is caused by S. maltophilia. In certain embodiments, the bacterial infection is caused by B. cepacia. In certain embodiments, the bacterial infection is caused by K. oxytoca.

In certain embodiments, the bacterial infection is caused by an Enterobacter, Pasteurella, Proteus, or Citrobacter species. Examples of species are provided herein.

In certain embodiments, the bacterial infection is an antibiotic-resistant bacterial infection, i.e., a bacterial infection caused by antibiotic-resistant bacteria. In certain embodiments, the bacterial infection is a Gram-negative, antibiotic-resistant bacterial infection. Therefore, also provided herein are methods for treating an antibiotic-resistant, Gram-negative bacterial infection in a subject comprising administering to the subject a peptide (e.g., StAMP) described herein, or a pharmaceutically acceptable salt thereof, or a pharmaceutical composition thereof. In certain embodiments, the antibiotic-resistant bacterial infection is caused by a bacteria resistant to one or more antibiotics selected from the group consisting of polymyxins, aminoglycosides, cephalosporins, penicillins, fluoroquinolones, tetracyclines, and β-lactams. In certain embodiments, the antibiotic-resistant bacterial infection is caused by a bacteria resistant to one or more antibiotics selected from the group consisting of carbapenem, vancomycin, methicillin, clarithromycin, ampicillin, ceftazidime, ceftriaxone, ciprofloxacin, gentamicin, and tobramycin.

As discussed herein, the present disclosure relates in part to antimicrobial peptides that have reduced toxicity as compared to known or existing antimicrobial agents (e.g., AMPs, e.g., StAMPs). The reduced toxicity can be, but is not limited to, reduced renal toxicity, reduced hepatic toxicity, and/or reduced hemolytic activity.

In certain embodiments, a peptide (e.g., StAMP) provided herein has reduced renal (i.e., kidney) toxicity as compared to a reference. Renal toxicity is reduced if the peptide kills and/or damages fewer kidney cells as compared to the reference. In certain embodiments, the peptide has reduced renal toxicity as compared to pexiganan. In certain embodiments, the peptide has reduced renal toxicity as compared to Esculentin-1A or a fragment thereof.

In some embodiments, a peptide provided herein has renal toxicity that is at least 10% (e.g., at least 15%, at least 20%, at least 25%, at least 30%, at least 35%, at least 40%, at least 45%, at least 50%, at least 55%, at least 60%, at least 65%, at least 65%, at least 70%, at least 75%, at least 80%, at least 85%, at least 90%, at least 95%, at least 99%) lower than the renal toxicity of the reference. The renal toxicity of a peptide can be measured in vitro using a renal proximal tubule epithelial cell (RPTEC) renal toxicity assay.

In certain embodiments a peptide (e.g., StAMP) provided herein has reduced hepatic (i.e., liver) toxicity as compared to a reference. Liver toxicity is reduced if the peptide kills and/or damages fewer liver cells as compared to the reference. In certain embodiments, the peptide has reduced hepatic toxicity as compared to pexiganan. In certain embodiments, the peptide has reduced hepatic toxicity as compared to Esculentin-1A or a fragment thereof.

In some embodiments, a peptide provided herein has hepatic toxicity that is at least 10% (e.g., at least 15%, at least 20%, at least 25%, at least 30%, at least 35%, at least 40%, at least 45%, at least 50%, at least 55%, at least 60%, at least 65%, at least 65%, at least 70%, at least 75%, at least 80%, at least 85%, at least 90%, at least 95%, at least 99%) lower than the hepatic toxicity of the reference. The hepatic toxicity of a peptide can be measured in vitro using a human liver cells (e.g., HepG2) toxicity assay.

In certain embodiments a peptide (e.g., StAMP) provided herein has reduced hemolytic activity as compared to a reference. “Hemolytic activity” for the purposes of this disclosure refers to the breakdown or lysis of red blood cells. In certain embodiments, the peptide has reduced hemolytic activity as compared to melittin. In certain embodiments, the peptide has reduced hemolytic activity as compared to Esculentin-1A or a fragment thereof.

In some embodiments, a peptide provided herein has hemolytic activity that is at least 10% (e.g., at least 15%, at least 20%, at least 25%, at least 30%, at least 35%, at least 40%, at least 45%, at least 50%, at least 55%, at least 60%, at least 65%, at least 65%, at least 70%, at least 75%, at least 80%, at least 85%, at least 90%, at least 95%, at least 99%) lower than the hemolytic activity of the reference.

Also provided herein are method of killing and/or inhibiting the growth of bacteria (e.g., Gram-negative and/or Gram-positive bacteria) comprising contacting the bacteria (e.g., in vitro or in vivo) with a peptide (e.g., StAMP) provided herein, or a pharmaceutically acceptable salt thereof, or a pharmaceutical composition thereof.

In certain embodiments, the bacteria are Gram-positive bacteria. In certain embodiments, the bacteria are Gram-negative bacteria. In certain embodiments, the Gram-negative bacteria are Acinetobacter, Escherichia, Pseudomonas, Neisseria, Chlamydia, Yersinia, Proteus, Enterobacter, Serratia, Helicobacter, Salmonella, Shigella, Moraxella, Stenotrophomonas, Bdellovibrio, Klebsiella, Legionella, or acetic acid bacteria. In certain embodiments, the bacteria are Acinetobacter baumannii, Escherichia coli, Pseudomonas aeruginosa, Neisseria gonorrhoeae, Chlamydia trachomatis, Yersinia pestis, Proteus mirabilis, Enterobacter cloacae, Serratia marcescens, Helicobacter pylori, Salmonella enteritidis, Salmonella typhi, Shigella, Klebsiella pneumoniae, or Legionella pneumophila bacteria, or a Gram-negative bacterium that contains a MCR-1 plasmid.

In certain embodiments, the bacteria are an Escherichia, Acinetobacter, Pseudomonas aeruginosa, or Klebsiella species. In certain embodiments, the bacteria are Escherichia coli (E. coli), Acinetobacter baumannii (A. baumannii), Pseudomonas aeruginosa (P. aeruginosa), or Klebsiella pneumoniae (K. pneumoniae). In certain embodiments, the bacteria are a Stenotrophomonas, Burkholderia, or Klebsiella species. In certain embodiments, the bacteria are Stenotrophomonas maltophilia (S. maltophilia), Burkholderia cepacia (B. cepacia), or Klebsiella oxytoca (K. oxytoca). In certain embodiments, the bacteria are E. coli. In certain embodiments, the bacteria are A. baumannii. In certain embodiments, the bacteria are P. aeruginosa. In certain embodiments, the bacteria are K. pneumoniae. In certain embodiments, the bacteria are S. maltophilia. In certain embodiments, the bacteria are B. cepacia. In certain embodiments, the bacteria are K. oxytoca.

In certain embodiments, the bacteria are an Enterobacter, Pasteurella, Proteus, or Citrobacter species. Examples of species are provided herein.

In certain embodiments, the bacteria are antibiotic-resistant bacteria. In certain embodiments, the bacteria are Gram-negative, antibiotic-resistant bacteria. Therefore, also provided herein are methods of killing and/or inhibiting the growth (e.g., in vitro or in vivo) of antibiotic-resistant, Gram-negative bacteria with a peptide (e.g., StAMP) described herein, or a pharmaceutically acceptable salt thereof, or a pharmaceutical composition thereof. In certain embodiments, the antibiotic-resistant bacteria are resistant to one or more antibiotics selected from the group consisting of polymyxins, aminoglycosides, cephalosporins, penicillins, fluoroquinolones, tetracyclines, and β-lactams. In certain embodiments, the antibiotic-resistant bacteria are resistant to one or more antibiotics selected from the group consisting of carbapenem, vancomycin, methicillin, clarithromycin, ampicillin, ceftazidime, ceftriaxone, ciprofloxacin, gentamicin, and tobramycin.

In certain embodiments, a peptide (e.g., StAMP) has a minimum inhibitory concentration (MIC) less than about 64 μg/mL. In certain embodiments, a peptide (e.g., StAMP) has a minimum inhibitory concentration (MIC) less than or equal to about 32 μg/mL, less than or equal to about 16 μg/mL, less than or equal to about 14 μg/mL, less than or equal to about 12 μg/mL, or less than or equal to about 10 μg/mL. In certain embodiments, a peptide (e.g., StAMP) has a minimum inhibitory concentration (MIC) less than or equal to about 10 μg/mL. In certain embodiments, a peptide (e.g., StAMP) has a MIC less than or equal to about 10 μg/mL, less than or equal to about 9 μg/mL, less than or equal to about 8 μg/mL, less than or equal to about 7 μg/mL, less than or equal to about 6 μg/mL, less than or equal to about 5 μg/mL, less than or equal to about 4 μg/mL, less than or equal to about 3 μg/mL, less than or equal to about 2 μg/mL, less than or equal to about 1 μg/mL. In certain embodiments, a peptide (e.g., StAMP) has a MIC of about 2 μg/mL. In certain embodiments, the MIC is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, or 1.9 μg/mL.

In certain embodiments, a peptide (e.g., StAMP) has a MIC of from about 0.1 to about 64 μg/mL, from about 0.1 to about 32 μg/mL, from about 0.1 to about 16 μg/mL, from about 0.1 to about 14 μg/mL, from about 0.1 to about 12 μg/mL, or from about 0.1 to about 10 μg/mL. In certain embodiments, a peptide (e.g., StAMP) has a MIC of from about 0.1 to about 10 μg/mL.

In certain embodiments, a peptide (e.g., StAMP) has a MIC of from about 0.1 to about 9 μg/mL, from about 0.1 to about 8 μg/mL, from about 0.1 to about 7 μg/mL, from about 0.1 to about 6 μg/mL, from about 0.1 to about 5 μg/mL, from about 0.1 to about 4 μg/mL, from about 0.1 to about 3 μg/mL, from about 0.1 to about 2 μg/mL. In certain embodiments, a peptide (e.g., StAMP) has a MIC of from about 0.1 to about 4 μg/mL. In certain embodiments, the MIC is at least about 0.2, 0.3, 0.4, 0.5, 0.6, 0.7, 0.8, 0.9, 1, 1.1, 1.2, 1.3, 1.4, 1.5, 1.6, 1.7, 1.8, or 1.9 μg/mL.

In certain embodiments, a peptide (e.g., StAMP) described herein selectively kills and/or inhibits the growth of Gram-negative bacteria over Gram-positive bacteria. In certain embodiments, the peptide selectively kills and/or inhibits the growth of Escherichia coli (E. coli), Acinetobacter baumannii (A. baumannii), Pseudomonas aeruginosa (P. aeruginosa), and/or Klebsiella pneumoniae (K. pneumoniae), over Staphylococcus aureus (S. aureus).

In certain embodiments, peptides (e.g., StAMPs) provided herein have the ability to kill and/or inhibit the growth of microbial cells but not mammalian cells. In certain embodiments, peptides (e.g., StAMPs) provided herein have the ability to selectively lyse microbial cells (e.g., bacterial cells) over mammalian cells. In certain embodiments, the peptides (e.g., StAMPs) are selectively cytotoxic to microbial cells (e.g., bacterial cells) over mammalian cells. This selectively can lead to reduced toxicity (e.g., reduced renal toxicity, hepatic toxicity, hemolytic activity) when the peptide is administered to a subject.

Provided herein are methods of selectively killing and/or inhibiting the growth of microbial cells (e.g., bacterial cells) over mammalian cells comprising contacting the microbial (e.g., bacterial) and mammalian cells with a peptide (e.g., StAMP) provided herein, or a pharmaceutically acceptable salt thereof, or a pharmaceutical composition thereof. In certain embodiments, peptides (e.g., StAMPs) provided herein lyse microbial cells (e.g., bacterial cells) to a greater extent than mammalian cells. Therefore, provided herein are methods of selectively lysing microbial cells (e.g., bacterial cells) over mammalian cells comprising contacting the microbial (e.g., bacterial) and mammalian cells with a peptide (e.g., StAMP) provided herein, or a pharmaceutically acceptable salt thereof, or a pharmaceutical composition thereof.

In certain embodiments, the microbial cells are bacterial cells. In certain embodiments, the microbial cells are Gram-negative bacterial cells. The cells can be of any bacteria (e.g., Gram-negative bacteria) provided herein. In certain embodiments, the bacterial cells are Escherichia, Acinetobacter, Pseudomonas aeruginosa, or Klebsiella cells. In certain embodiments, the bacterial cells are Escherichia coli (E. coli), Acinetobacter baumannii (A. baumannii), Pseudomonas aeruginosa (P. aeruginosa), or Klebsiella pneumoniae (K. pneumoniae) cells. In certain embodiments, the bacterial cells are Stenotrophomonas, Burkholderia, or Klebsiella cells. In certain embodiments, the bacterial cells are Stenotrophomonas maltophilia (S. maltophilia), Burkholderia cepacia (B. cepacia), or Klebsiella oxytoca (K. oxytoca) cells. In certain embodiments, the bacterial cells are E. coli cells. In certain embodiments, the bacterial cells are A. baumannii cells. In certain embodiments, the bacterial cells are P. aeruginosa cells. In certain embodiments, the bacterial cells are K. pneumoniae cells. In certain embodiments, the bacterial cells are S. maltophilia cells. In certain embodiments, the bacterial cells are B. cepacia cells. In certain embodiments, the bacterial cells are K. oxytoca cells.

In certain embodiments, the bacterial cells are Enterobacter, Pasteurella, Proteus, or Citrobacter cells. Examples of species are provided herein.

Also provided here are peptides (e.g., StAMPs) described herein, and pharmaceutically acceptable salts thereof, and pharmaceutical compositions thereof, for use in any of the methods described herein (e.g., for treating an infectious disease in a subject, treating a bacterial infection in a subject, killing and/or inhibiting the growth of bacteria in a subject, selectively killing and/or inhibiting the growth of bacterial cells over mammalian cells in a subject, etc.)

In another aspect, also provided herein are uses of peptides (e.g., StAMPs) described herein, and pharmaceutically acceptable salts thereof, and pharmaceutical compositions thereof, for the manufacture of medicament (e.g., for treating an infectious disease in a subject, treating a bacterial infection in a subject, killing and/or inhibiting the growth of bacteria in a subject, selectively killing and/or inhibiting the growth of bacterial cells over mammalian cells in a subject, etc.)

In certain embodiments, in vivo (i.e., in a subject) methods and uses provided herein comprise administering to a subject an effective amount of a peptide (e.g., StAMP) provided herein, or a pharmaceutically acceptable salt thereof, or a pharmaceutical composition thereof. In certain embodiments, the effective amount is a therapeutically effective amount. In certain embodiments, the effective amount is a prophylactically effective amount.

In certain embodiments, in vitro methods and uses provided herein can be carried out, e.g., in a cellular assay or biological sample. In certain embodiments, in vitro methods and uses comprise contacting a microbial cell (e.g., bacterial cell) with a peptide provided herein, or a pharmaceutically acceptable salt thereof, or a pharmaceutical composition thereof.

### EXAMPLES

### General Methods

Solid phase peptide synthesis: Fmoc-based solid-phase peptide synthesis was used to synthesize the antimicrobial peptides and their stapled derivatives. To achieve the i+4 staple lengths, cysteines were used flanking three residues. To achieve the i+7 staple lengths, cysteines were used flanking six residues. The peptides were cleaved off the resin using trifluoroacetic acid, then precipitated using a hexane:ether (1:1) mixture, and afterwards purified using a prep HPLC. Final peptide characterization for purity was assessed using a UHPLC/MS system. Eluate solutions containing peptides with a purity of greater than about 80% were lyophilized.

Thiol-halide stapling: For i+4 and i+7 thiol-halide staples, lyophilized peptides were dissolved in an aqueous buffered solution. A solution of the appropriate crosslinking reagent (e.g., from Table 4, to form mxy, pxy, but, bbf, bbp, or bbn) was added in ⅓-2× the volume of the peptide solution. The solution was stirred at room temperature before lyophilizing. Stapled peptides were purified by preparative HPLC and their final purity was assessed by UHPLC/MS.

Thiol-perfluoroarene staple: For thiol-perfluoroarene staples, lyophilized peptide was dissolved in of N,N-dimethylformamide containing 40 mM tris base. To this solution was added tris-2-carboxyethylphosphine hydrochloride (TCEP), guanidine hydrochloride, and perfluoroarene linker, all dissolved in N,N-dimethylformamide with 40 mM tris base. The reaction was stirred overnight, then purified by preparative HPLC. Final peptide purity was assessed by UHPLC-MS.

Antimicrobial Activity Assay: The following microbroth dilution protocol was adapted from CLSI to determine the minimum inhibitory concentration (MIC) of StAMPs. Briefly, Mueller-Hinton broth (MHB) or cation-adjusted Mueller-Hinton broth (MHB II) was prepared and autoclaved. Then, a colony of bacterial cells was picked and grown overnight in broth at 37° C. and then diluted and allowed to grow again for 3-4 hours. Serial dilutions of peptide stocks in water (10 μl) were prepared using clear round-bottom polypropylene 96-well plates. Then 90 μl of bacteria in broth was added to give a final inoculum of 5×10⁵ CFU/ml. The plates were then covered with porous tape to reduce evaporation and incubated for 20-24 hours at 37° C. The MIC was the minimum peptide concentration at which no visible bacterial growth was observed.

Cell culture: RPTEC were maintained in MEM media supplemented with growth factors. HEPG2 cell line was maintained in Eagle's Minimum Essential Medium (EMEM) supplemented with fetal bovine serum to a final concentration of 10%.

90 minute cytotoxicity assay: Cells were plated in a 96-well format, and after overnight incubation, media was replaced with fresh media. Serial dilutions of StAMPs from a 5 mg/mL water stock, or vehicle, were added to the cells in a final volume of 100 μl. After incubating at 37° C. for 90 min, 50 μl of cell culture media was transferred to a clear 96-well plate, incubated with 50 μl of lactate dehydrogenase (LDH) assay reagent for 10 min, and absorbance measured at 490 nm on a microplate reader.

48 hours cytotoxicity assay: Cells were plated in a 96-well format, and after 4 hour incubation to allow attachment, serial dilutions of StAMPs from a 5 mg/mL water stock, or vehicle, were then added to the cells in a final volume of 100 μl. After incubating at 37° C. for 48 hours, 100 μl of CellTiter-Glo® reagent was added to the cells, and the plates were incubated 10 minutes at room temperature. Luminescence was then measured on a microplate reader.

### Antimicrobial Activity and Toxicity

As shown in Tables A-C, antimicrobial peptides (e.g., StAMPs) provided herein exhibit broad-spectrum Gram-negative antimicrobial activity and are selective for bacterial cells over renal and hepatic cells. Renal toxicity was measured after 90 minutes of incubation, while liver toxicity was measured after 48 hours of incubation with StAMPs. N/A=not tested.

The following apply to all peptides (i.e., StAMPs) listed in Tables A-C: N-terminus: —NH₂; C-terminus: amidated with —NH₂ (unless otherwise indicated); counterion: Cl⁻.

TABLE A
Antimicrobial Activity and Renal/Hepatic Toxicity

| | | | | | | | | Inhibitory Concentration | |
|---|---|---|---|---|---|---|---|---|---|
| | | | | | | | | at 50% Cytotoxicity | |
| | | | | | | | | (IC50) [μg/mL] | |
| | | | Minimum Inhibitory Concentration | | | | | Human Renal | Human |
| | SEQ | | (MIC) [μg/mL] | | | | | Proximal Tubule | Liver |
| StAMP | ID | X¹-X² | E. | A. | P. | K. | S. | Epithelial Cells | Cells |
| # | NO: | Crosslink | coli | baumannii | aeruginosa | pneumoniae | aureus | (RPTEC) | (HepG2) |
| 1 | 3 | but | 8 | 8 | 32 | 64 | >64 | >200 | >200 |
| 2 | 7 | mxy | 2 | 4 | 8 | 16 | >64 | 101 | 97 |
| 3 | 7 | pxy | 2 | 2 | 8 | 16 | >64 | 79 | 86 |
| 4 | 7 | but | 4 | 8 | 16 | 64 | >64 | ~200 | 98 |
| 5 | 4 | mxy | 4 | 4 | 16 | 6 | 32 | 14 | 23 |
| 6 | 4 | pxy | 4 | 4 | 16 | 8 | 32 | 15 | 25 |
| 7 | 4 | but | 4 | 4 | 32 | 32 | >64 | 62 | 51 |
| 8 | 6 | pxy | 4 | 4 | 16 | 32 | >64 | 119 | 15 |
| 9 | 6 | but | 32 | 32 | 64 | >64 | >64 | >200 | 39 |
| 10 | 8 | mxy | 4 | 4 | 16 | 8 | 32 | 17 | 18 |
| 11 | 8 | pxy | 4 | 4 | 8 | 8 | 32 | 16 | 23 |
| 12 | 8 | but | 4 | 4 | 16 | 16 | >64 | 70 | 50 |
| 13 | 9 | pxy | 32 | 32 | 64 | 64 | >64 | >200 | >200 |
| 14 | 10 | mxy | 4 | 8 | 16 | 16 | 32 | 41 | 49 |
| 15 | 10 | pxy | 8 | 16 | 32 | 32 | >64 | 82 | 88 |
| 16 | 10 | but | 32 | 64 | >64 | >64 | >64 | >200 | >200 |
| 17 | 11 | mxy | 64 | >64 | >64 | >64 | >64 | >200 | >200 |
| 18 | 11 | pxy | >64 | >64 | >64 | >64 | >64 | >200 | >200 |
| 19 | 11 | but | >64 | >64 | >64 | >64 | >64 | >200 | >200 |
| 20 | 12 | mxy | 8 | 8 | 16 | 16 | 64 | 25 | 40 |
| 21 | 12 | pxy | 8 | 16 | 32 | 32 | >64 | 96 | 200 |
| 22 | 12 | but | 32 | 64 | 64 | >64 | >64 | >200 | >200 |
| 23 | 13 | mxy | 4 | 4 | 4 | 4 | 32 | 52 | 97 |
| 24 | 13 | pxy | 4 | 4 | 8 | 8 | 32 | 54 | 103 |
| 25 | 13 | but | 4 | 2 | 8 | 32 | >64 | 200 | 141 |
| 26 | 14 | mxy | 2 | 2 | 8 | 8 | >64 | 64 | 44 |
| 27 | 14 | pxy | 64 | 32 | 64 | >64 | >64 | >200 | >200 |
| 28 | 14 | but | 8 | 8 | 32 | >64 | >64 | >200 | >200 |
| 29 | 15 | mxy | 64 | 16 | 64 | >64 | >64 | >200 | ~200 |
| 30 | 15 | pxy | 64 | 16 | 64 | >64 | >64 | >200 | >200 |
| 31 | 15 | but | >64 | >64 | >64 | >64 | >64 | >200 | >200 |
| 32 | 16 | mxy | 2 | 2 | 16 | 8 | >64 | 30 | 41 |
| 33 | 16 | pxy | 4 | 4 | 16 | 8 | 64 | 59 | 57 |
| 34 | 16 | but | 8 | 3 | 32 | 64 | >64 | ~200 | 119 |
| 35 | 17 | but | 4 | 4 | 16 | 32 | >64 | ~200 | ~200 |
| 36 | 1 | mxy | >64 | 64 | >64 | >64 | >64 | ~200 | 75 |
| 37 | 1 | pxy | 64 | 32 | >64 | >64 | >64 | 140 | 33 |
| 38 | 1 | but | >64 | >64 | >64 | >64 | >64 | >200 | >200 |
| 39 | 3 | mxy | 8 | 8 | 32 | 32 | >64 | 113 | 103 |
| 40 | 3 | pxy | 8 | 8 | 32 | 32 | >64 | 117 | 124 |
| 41 | 6 | mxy | 8 | 8 | 32 | 32 | >64 | 58 | 21 |
| 42 | 9 | mxy | 16 | 8 | 32 | 32 | >64 | >200 | >200 |
| 43 | 9 | but | 32 | 32 | >64 | >64 | >64 | >200 | >200 |
| 44 | 17 | mxy | 4 | 4 | 16 | 32 | 32 | 86 | 48 |
| 45 | 17 | pxy | 4 | 4 | 16 | 16 | 64 | 105 | 83 |
| 46 | 2 | mxy | 8 | 8 | 64 | 64 | >64 | N/A | 201 |
| 47 | 2 | pxy | 8 | 8 | 64 | 64 | >64 | N/A | 402 |
| 48 | 2 | but | 64 | 64 | >64 | >64 | >64 | N/A | >800 |
| 91* | 7 | mxy | 64 | 32 | >64 | >64 | >64 | >200 | N/A |
| 92* | 74 | mxy | 8 | 8 | 64 | >64 | >64 | N/A | N/A |
| 93* | 65 | mxy | N/A | N/A | N/A | N/A | N/A | N/A | N/A |
| 94* | 66 | mxy | 12 | 8 | 64 | >64 | >64 | 168 | >200 |
| 95* | 67 | mxy | 12 | 8 | 64 | 64 | >64 | 167 | >200 |
| 96* | 68 | mxy | 8 | 4 | 64 | 64 | >64 | 153 | 193 |
| 97* | 69 | mxy | 16 | 8 | 64 | >64 | >64 | >200 | >200 |
| 98* | 70 | mxy | 1 | 1 | 32 | 8 | >64 | 28 | 50 |
| 99* | 77 | mxy | 2 | 2 | 64 | 16 | >64 | 45 | 59 |
| 100* | 68 | but | 32 | 16 | >64 | >64 | >64 | N/A | >200 |
| 101* | 78 | but | >64 | >64 | >64 | >64 | >64 | N/A | 106 |
| 102 | 78 | but | >64 | >64 | >64 | >64 | >64 | N/A | 108 |
| 103** | 78 | but | >64 | >64 | >64 | >64 | >64 | N/A | N/A |

*C-terminus: —COOH
**C-terminus: —NHOH

TABLE B
Antimicrobial Activity and Renal/Hepatic Toxicity

| | | | | | | | | Inhibitory Concentration | |
|---|---|---|---|---|---|---|---|---|---|
| | | | | | | | | at 50% Cytotoxicity | |
| | | | | | | | | (IC50) [μg/mL] | |
| | | | Minimum Inhibitory Concentration | | | | | Human Renal | Human |
| | SEQ | | (MIC) [μg/mL] | | | | | Proximal Tubule | Liver |
| StAMP | ID | X¹-X² | E. | A. | P. | K. | S. | Epithelial Cells | Cells |
| # | NO: | Crosslink | coli | baumannii | aeruginosa | pneumoniae | aureus | (RPTEC) | (HepG2) |
| 49 | 18 | bbp | 32 | 32 | >64 | >64 | >64 | 60 | 22 |
| 50 | 18 | bbn | 16 | 8 | >64 | 32 | 64 | 17 | 103 |
| 51 | 18 | bbf | 16 | 8 | >64 | >64 | >64 | N/A | 102 |
| 52 | 19 | bbn | 32 | 32 | >64 | >64 | >64 | >200 | 10 |
| 53 | 19 | bbf | 16 | 4 | >64 | >64 | >64 | 40 | 78 |
| 54 | 20 | bbp | 8 | 8 | 64 | 16 | 64 | 81 | 43 |
| 55 | 20 | bbn | 8 | 8 | 32 | 16 | 32 | 23 | 53 |
| 56 | 20 | bbf | 8 | 4 | 32 | 16 | 8 | 17 | 34 |
| 57 | 21 | bbp | 16 | 16 | >64 | >64 | >64 | >200 | 103 |
| 58 | 21 | bbn | 16 | 4 | 64 | 32 | >64 | 131 | 51 |
| 59 | 21 | bbf | 8 | 4 | 32 | 8 | 32 | N/A | 23 |
| 60 | 22 | bbp | >64 | >64 | >64 | >64 | >64 | N/A | 29 |
| 61 | 22 | bbf | 16 | 8 | >64 | 64 | 16 | N/A | >200 |
| 62 | 22 | bbn | 16 | 4 | >64 | >64 | >64 | N/A | 81 |
| 63 | 23 | bbp | 8 | 8 | >64 | 12 | >64 | N/A | 44 |
| 64 | 23 | bbn | 8 | 8 | >64 | 16 | >64 | N/A | 53 |
| 65 | 23 | bbf | 4 | 4 | 16 | 4 | 32 | 15 | 13 |
| 66 | 24 | bbp | 8 | 4 | 32 | 16 | >64 | N/A | 75 |
| 67 | 24 | bbn | 4 | 4 | 32 | 8 | >64 | N/A | 59 |
| 68 | 24 | bbf | 4 | 4 | 8 | 4 | 32 | N/A | 8 |
| 69 | 25 | bbn | 16 | 16 | >64 | 32 | >64 | N/A | 52 |
| 70 | 25 | bbf | >64 | 64 | >64 | >64 | >64 | N/A | 23 |
| 71 | 25 | bbp | 64 | 32 | >64 | >64 | >64 | N/A | 360 |
| 72 | 26 | bbp | 4 | 4 | 32 | 8 | 4 | 19 | N/A |
| 73 | 26 | bbn | 8 | 4 | 64 | 8 | 4 | 9 | N/A |
| 74 | 26 | bbf | 32 | 8 | 64 | 32 | 8 | 4 | N/A |
| 75 | 27 | bbn | 4 | 4 | 16 | 16 | 64 | N/A | 98 |
| 76 | 27 | bbf | 4 | 4 | 16 | 4 | 32 | N/A | 23 |
| 77 | 27 | bbp | 4 | 4 | 32 | 32 | >64 | N/A | 159 |
| 78 | 28 | bbn | 16 | 16 | 64 | 64 | >64 | N/A | 108 |
| 79 | 28 | bbf | 8 | 4 | 32 | 8 | 32 | N/A | 50 |
| 80 | 28 | bbp | 32 | 32 | >64 | >64 | >64 | N/A | 49 |
| 81 | 29 | bbp | 4 | 4 | 32 | 8 | >64 | N/A | 57 |
| 82 | 29 | bbn | 4 | 4 | 32 | 8 | >64 | N/A | 23 |
| 83 | 29 | bbf | 8 | 4 | 32 | 8 | 16 | N/A | N/A |
| 84 | 30 | bbp | 4 | 8 | 32 | 16 | >64 | N/A | 105 |
| 85 | 30 | bbn | 4 | 4 | 16 | 32 | 64 | N/A | 87 |
| 86 | 30 | bbf | 4 | 4 | 16 | 4 | 32 | N/A | 9 |
| 87 | 31 | bbn | 4 | 4 | 8 | 8 | 64 | 44 | 54 |
| 88 | 31 | bbf | 4 | 4 | 8 | 4 | 32 | 13 | 15 |
| 89 | 31 | bbp | 4 | 4 | 32 | 16 | >64 | 80 | 62 |

TABLE C
Antimicrobial Activity and Renal/Hepatic Toxicity

| | | | | | | | | Inhibitory Concentration | |
|---|---|---|---|---|---|---|---|---|---|
| | | | | | | | | at 50% Cytotoxicity | |
| | | | | | | | | (IC50) [μg/mL] | |
| | | X¹-X² | Minimum Inhibitory Concentration | | | | | Human Renal | Human |
| | SEQ | and | (MIC) [μg/mL] | | | | | Proximal Tubule | Liver |
| StAMP | ID | X³-X⁴ | E. | A. | P. | K. | S. | Epithelial Cells | Cells |
| # | NO: | Crosslink | coli | baumannii | aeruginosa | pneumoniae | aureus | (RPTEC) | (HepG2) |
| 90 | 32 | pxy | 8 | 4 | 32 | 64 | >64 | >200 | N/A |

### Additional Embodiments

Additional embodiments of the disclosure are indicated by the following numbered paragraphs:

1. A peptide, or a pharmaceutically acceptable salt thereof, comprising 10 or more contiguous amino acids of Esculentin-1A and comprising exactly one internal crosslink connecting two crosslinked amino acids, wherein:
two amino acids of the 10 or more contiguous amino acids of Esculentin-1A are independently substituted by X¹ and X²; and
X¹ and X² are independently the crosslinked amino acids;
optionally wherein the amino acid sequence includes 1 to 3 amino acid substitutions, inclusive, at positions other than X¹ and X².
   2. A peptide, or a pharmaceutically acceptable salt thereof, comprising 10 or more contiguous amino acids of Esculentin-1A; wherein:
exactly two amino acids of the 10 or more contiguous amino acids of Esculentin-1A are independently substituted by X¹ and X²; and
X¹ and X² are each independently amino acids comprising reactive moieties capable of forming a crosslink with the other;
optionally wherein the amino acid sequence includes 1 to 3 amino acid substitutions, inclusive, at positions other than X¹ and X².
   3. The peptide of paragraph 1 or 2, or a pharmaceutically acceptable salt thereof, comprising 20 or more contiguous amino acids of Esculentin-1A; optionally wherein the amino acid sequence includes 1 to 6 amino acid substitutions, inclusive, at positions other than X¹ and X².
   4. The peptide of any one of paragraphs 1-3, wherein X¹ and X² are separated by 3 amino acids, forming an i+4 crosslink.
   5. The peptide of any one of paragraphs 1-3, wherein X¹ and X² are separated by 6 amino acids, forming an i+7 crosslink.
   6. The peptide of paragraph 1, wherein the peptide comprises the following amino acid sequence:

(SEQ ID NO: 7)
G I F S K L X¹ G K K X² K N L L I S G L K G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².
   7. The peptide of paragraph 6, wherein the peptide comprises the following amino acid sequence:

(SEQ ID NO: 74)
G I F S K L X¹ G K K X² K N L L I S G L K G G G,

or a pharmaceutically acceptable salt thereof.
8. The peptide of paragraph 6, wherein the peptide comprises one of the following amino acid sequences:

(SEQ ID NO: 65)
G I F S K L X¹ G K K X² K N L L I S G L K G G G G,
(SEQ ID NO: 66)
G I F S K L X¹ G K K X² K N L L I S G L K G G G K,
(SEQ ID NO: 67)
G I F S K L X¹ G K K X² K N L L I S G L K G A G,
(SEQ ID NO: 68)
G I F S K L X¹ G K K X² K N L L I S G L K G A K,
(SEQ ID NO: 69)
G I F S K L X¹ A K K X² K N L L I S G L K G,
(SEQ ID NO: 70)
G I F S K L X¹ G K K X² K N L L I S A L K G,
(SEQ ID NO: 77)
G I F S K L X¹ A K K X² K N L L I S A L K G,

or a pharmaceutically acceptable salt thereof.
9. The peptide of paragraph 1, wherein the peptide comprises one of the following amino acid sequences:

(SEQ ID NO: 1)
X¹ I F S X² L A G K K I K N L L I S G L K G,
(SEQ ID NO: 2)
G X¹ F S K X² A G K K I K N L L I S G L K G,
(SEQ ID NO: 3)
G I X¹ S K L X² G K K I K N L L I S G L K G,
(SEQ ID NO: 4)
G I F X¹ K L A X² K K I K N L L I S G L K G,
(SEQ ID NO: 5)
G I F S X¹ L A G X² K I K N L L I S G L K G,
(SEQ ID NO: 6)
G I F S K X¹ A G K X² I K N L L I S G L K G,
(SEQ ID NO: 7)
G I F S K L X¹ G K K X² K N L L I S G L K G,
(SEQ ID NO: 8)
G I F S K L A X¹ K K I X² N L L I S G L K G,
(SEQ ID NO: 9)
G I F S K L A G X¹ K I K X² L L I S G L K G,
(SEQ ID NO: 10)
G I F S K L A G K X¹ I K N X² L I S G L K G,
(SEQ ID NO: 11)
G I F S K L A G K K X¹ K N L X² I S G L K G,
(SEQ ID NO: 12)
G I F S K L A G K K I X¹ N L L X² S G L K G,
(SEQ ID NO: 13)
G I F S K L A G K K I K X¹ L L I X² G L K G,
(SEQ ID NO: 14)
G I F S K L A G K K I K N X¹ L I S X² L K G,
(SEQ ID NO: 15)
G I F S K L A G K K I K N L X¹ I S G X² K G,
(SEQ ID NO: 16)
G I F S K L A G K K I K N L L X¹ S G L X² G,
(SEQ ID NO: 17)
G I F S K L A G K K I K N L L I X¹ G L K X²,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².
   10. The peptide of paragraph 9, wherein the peptide comprises the amino acid sequence: JIFSKXIAGKX²IKNLLISGLKG (SEQ ID NO: 78), or a pharmaceutically acceptable salt thereof.
   11. The peptide of paragraph 1, wherein the peptide comprises one of the following amino acid sequences:

(SEQ ID NO: 18)
X¹ I F S K L A X² K K I K N L L I S G L K G,
(SEQ ID NO: 19)
G X¹ F S K L A G X² K I K N L L I S G L K G,
(SEQ ID NO: 20)
G I X¹ S K L A G K X² I K N L L I S G L K G,
(SEQ ID NO: 21)
G I F X¹ K L A G K K X² K N L L I S G L K G,
(SEQ ID NO: 22)
G I F S X¹ L A G K K I X² N L L I S G L K G,
(SEQ ID NO: 23)
G I F S K X¹ A G K K I K X² L L I S G L K G,
(SEQ ID NO: 24)
G I F S K L A X¹ K K I K N L X² I S G L K G,
(SEQ ID NO: 25)
G I F S K L A G X¹ K I K N L L X² S G L K G,
(SEQ ID NO: 26)
G I F S K L A G K X¹ I K N L L I X² G L K G,
(SEQ ID NO: 27)
G I F S K L A G K K X¹ K N L L I S X² L K G,
(SEQ ID NO: 28)
G I F S K L A G K K I X¹ N L L I S G X² K G,
(SEQ ID NO: 29)
G I F S K L A G K K I K X¹ L L I S G L X² G,
(SEQ ID NO: 30)
G I F S K L A G K K I K N X¹ L I S G L K X²,
(SEQ ID NO: 31)
G I F S K L X¹ G K K I K N X² L I S G L K G,

or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².
   12. The peptide of paragraph 2, wherein the peptide comprises any one of SEQ ID NOs: 1-31, 65-70, 74, and 77;
optionally wherein the amino acid sequence includes 1 to 6 amino acid substitutions, inclusive, at positions other than X¹ and X².
   13. The peptide of any one of paragraphs 1-12, or a pharmaceutically acceptable salt thereof, wherein the amino acid sequence includes 1 to 3 amino acid substitutions, inclusive, at positions other than X¹ and X².
   14. The peptide of any one of paragraphs 1-12, or a pharmaceutically acceptable salt thereof, wherein the amino acid sequence includes 1 amino acid substitutions at a position other than X¹ or X².
   15. The peptide of any one of paragraphs 1-12, or a pharmaceutically acceptable salt thereof, wherein the amino acid sequence does not include an amino acid substitution.
   16. A peptide comprising the amino acid sequence:

(SEQ ID NO: 32)
G I F S K L X¹ G K K X² K N L L I X³ G L K X⁴,

or a pharmaceutically acceptable salt thereof, wherein:
X¹, X², X³, and X⁴ are independently amino acids;
X¹ and X² are connected via a crosslink, and X³ and X⁴ are connected via a crosslink; and
optionally wherein the amino acid sequence includes 1 to 6 amino acid substitutions, inclusive, at positions other than X¹, X², X³, and X⁴.
   17. A peptide comprising the amino acid sequence SEQ ID NO: 32, or a pharmaceutically acceptable salt thereof, wherein:
X¹, X², X³, and X⁴ are independently amino acids;
X¹ and X² each independently comprise a reactive moiety capable of forming a crosslink with the other, and X³ and X⁴ each independently comprise a reactive moiety capable of forming a crosslink with the other; and
the amino acid sequence includes 1 to 6 amino acid substitutions, inclusive, at positions other than X¹, X², X³, and X⁴.
   18. The peptide of any one of paragraphs 1-17, or a pharmaceutically acceptable salt thereof, wherein one or more instances of K are independently substituted by an amino acid selected from Orn, Dab, Dap, R, and hArg.
   19. The peptide of paragraph 18, or a pharmaceutically acceptable salt thereof, wherein each instance of K is independently substituted by an amino acid selected from Orn, Dab, Dap, R, and hArg.
   20. The peptide of any one of paragraphs 1-19, wherein one or more instances of F are independently substituted by an amino acid selected from F¹, F⁵, F⁴, F², and F³.
   21. The peptide of paragraph 20, or a pharmaceutically acceptable salt thereof, wherein each instance of F is independently substituted by an amino acid selected from F¹, F⁵, F⁴, F², and F³.
   22. The peptide of any one of paragraphs 1-21, or a pharmaceutically acceptable salt thereof, further comprising a small molecule, lipophilic group, or polymer conjugated to the C-terminus of the peptide.
   23. The peptide of paragraph 22, wherein the lipophilic group is a lipid or fatty acid.
   24. The peptide of paragraph 22, wherein the peptide comprises PEG conjugated to C-terminus, or wherein the peptide is amidated at the C-terminus with a group of the formula: —NH(CH₂CH₂O)₁₋₂₀CH₂CH₂CONH₂.
   25. The peptide of any one of paragraphs 1-22, or a pharmaceutically acceptable salt thereof, comprising an amino acid or peptide conjugated to the C-terminus of the peptide.
   26. The peptide of paragraph 25, or a pharmaceutically acceptable salt thereof, comprising one of the following amino acid sequences conjugated to the C-terminus of the peptide:

GE,
AG,
AA,
AK,
GG,
GGE,
GGS,
GGG,
GGK,
GGQ,
(SEQ ID NO: 88)
GGGE,
(SEQ ID NO: 89)
GGEE,
or
(SEQ ID NO: 90)
GGSGGS.

27. The peptide of paragraph 22, or a pharmaceutically acceptable salt thereof, comprising a polymyxin conjugated to the C-terminus of the peptide.
28. The peptide of any one of paragraphs 1-21, wherein the C-terminus is amidated.
29. The peptide of any one of paragraphs 1-28, wherein the C-terminus is amidated with —NH₂.
30. The peptide of any one of paragraphs 1-28, wherein the C-terminus is amidated with —NHOH.
31. The peptide of any one of paragraphs 1-30, or a pharmaceutically acceptable salt thereof, wherein the peptide is 100 amino acids or fewer in length.
32. The peptide of any one of paragraphs 1-31, or a pharmaceutically acceptable salt thereof, wherein the peptide is 30 amino acids or fewer in length.
33. The peptide of any one of the preceding paragraphs, or a pharmaceutically acceptable salt thereof, wherein the crosslinks are attached to the α-positions of the amino acids X¹, X², X³, and X⁴.
34. The peptide of any one of the preceding paragraphs, or a pharmaceutically acceptable salt thereof, wherein each crosslink is independently optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.
35. The peptide of any one of the preceding paragraphs, or a pharmaceutically acceptable salt thereof, wherein each crosslink is independently a dithio crosslink.
36. A peptide, or a pharmaceutically acceptable salt thereof, comprising 10 or more contiguous amino acids of Esculentin-1A and at least one internal dithio crosslink, wherein:
at least 2 amino acids of the 10 or more contiguous amino acids of Esculentin-1A are substituted by a pair of dithio-crosslinked amino acids;
optionally wherein the amino acid sequence includes 1 to 3 additional amino acid substitutions, inclusive.
   37. The peptide of paragraph 29, or a pharmaceutically acceptable salt thereof, comprising 20 or more contiguous amino acids of Esculentin-1A; wherein:
at least 2 amino acids of the 20 or more contiguous amino acids of Esculentin-1A are substituted by a pair dithio-crosslinked amino acids;
optionally wherein the amino acid sequence includes 1 to 6 additional amino acid substitutions, inclusive.
   38. The peptide of paragraph 36 or 37, or a pharmaceutically acceptable salt thereof, wherein the pair of dithio-crosslinked amino acids are separated by 3 amino acids, forming an i+4 dithio crosslink.
   39. The peptide of paragraph 36 or 37, or a pharmaceutically acceptable salt thereof, wherein the pair of dithio-crosslinked amino acids are separated by 6 amino acids, forming an i+7 dithio crosslink.
   40. The peptide of any of paragraphs 35-39, or a pharmaceutically acceptable salt thereof, wherein each dithio crosslink is independently of the following formula:

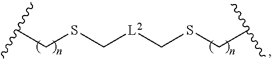

wherein each n is independently an integer from 1-10, inclusive;
L² is optionally substituted alkylene,

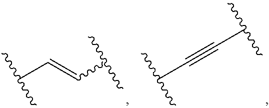

optionally substituted arylene, optionally substituted heteroarylene, or -A¹-A¹-; wherein each instance of A¹ is independently optionally substituted arylene or optionally substituted heteroarylene.
41. The peptide of paragraph 40, or a pharmaceutically acceptable salt thereof, wherein each dithio crosslink is independently of one of the following formulae:

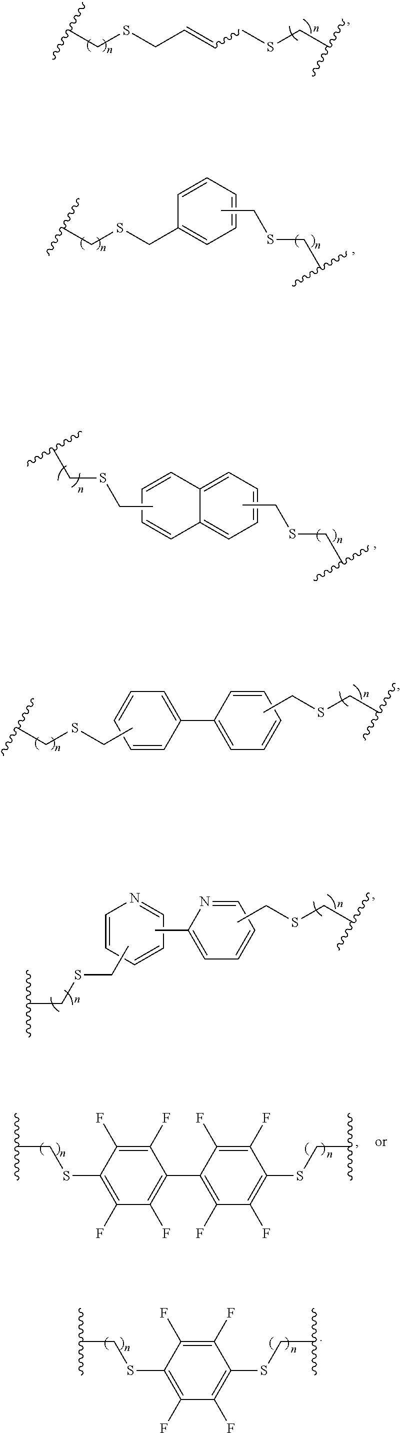

42. The peptide of paragraph 40 or 41, or a pharmaceutically acceptable salt thereof, wherein each instance of n is 1.
43. The peptide of any one of the preceding paragraphs, or a pharmaceutically acceptable salt thereof, wherein each crosslink is independently of one of the following formulae:

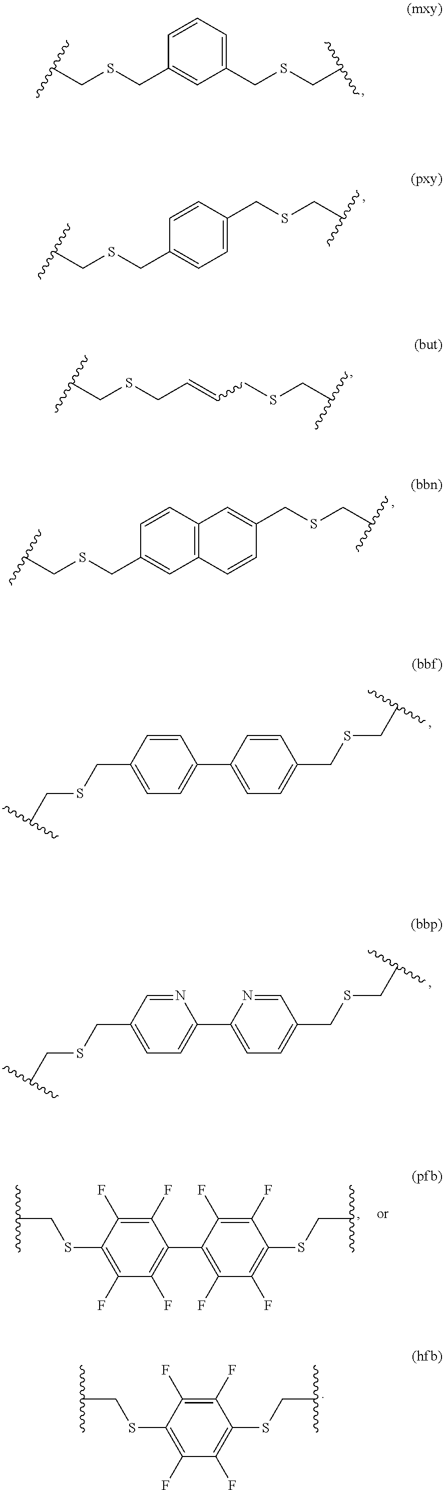

44. The peptide of any one of the preceding paragraphs, or a pharmaceutically acceptable salt thereof, wherein each crosslink is independently about 5 Å to about 35 Å in length, inclusive.
45. The peptide of any one of the preceding paragraphs, or a pharmaceutically acceptable salt thereof, wherein the length of each crosslink is approximately equal to the length of 5 to 25 carbon-carbon and/or carbon-sulfur bonds, inclusive.
46. The peptide of any one of the preceding paragraphs, or a pharmaceutically acceptable salt thereof, wherein at least one crosslink spans an α-helix of the peptide.
47. The peptide of any one of the preceding paragraphs, or a pharmaceutically acceptable salt thereof, wherein at least one crosslink stabilizes an α-helix of the peptide.
48. The peptide of any one of the preceding paragraphs, or a pharmaceutically acceptable salt thereof, wherein the peptide has increased α-helicity as compared to a corresponding uncrosslinked peptide.
49. The peptide of any one of the preceding paragraphs, or a pharmaceutically acceptable salt thereof, wherein the peptide comprises one of the following peptides:

| | | | | | | | | | | | | | | | | | | | | | | | | | SEQ | X¹-X² | |
|---|---|---|---|---|---|---|---|---|---|---|---|---|---|---|---|---|---|---|---|---|---|---|---|---|---|---|---|
| StAMP | | | | | | | | | | | | | | | | | | | | | | | | | ID | Cross- | C- |
| # | Sequence | | | | | | | | | | | | | | | | | | | | | | | | NO: | link | terminus |
| 1 | G | I | X¹ | S | K | L | X² | G | K | K | I | K | N | L | L | I | S | G | L | K | G | | | | 3 | but | —NH₂ |
| 2 | G | I | F | S | K | L | X¹ | G | K | K | X² | K | N | L | L | I | S | G | L | K | G | | | | 7 | mxy | —NH₂ |
| 3 | G | I | F | S | K | L | X¹ | G | K | K | X² | K | N | L | L | I | S | G | L | K | G | | | | 7 | pxy | —NH₂ |
| 4 | G | I | F | S | K | L | X¹ | G | K | K | X² | K | N | L | L | I | S | G | L | K | G | | | | 7 | but | —NH₂ |
| 5 | G | I | F | X¹ | K | L | A | X² | K | K | I | K | N | L | L | I | S | G | L | K | G | | | | 4 | mxy | —NH₂ |
| 6 | G | I | F | X¹ | K | L | A | X² | K | K | I | K | N | L | L | I | S | G | L | K | G | | | | 4 | pxy | —NH₂ |
| 7 | G | I | F | X¹ | K | L | A | X² | K | K | I | K | N | L | L | I | S | G | L | K | G | | | | 4 | but | —NH₂ |
| 8 | G | I | F | S | K | X¹ | A | G | K | X² | I | K | N | L | L | I | S | G | L | K | G | | | | 6 | pxy | —NH₂ |
| 9 | G | I | F | S | K | X¹ | A | G | K | X² | I | K | N | L | L | I | S | G | L | K | G | | | | 6 | but | —NH₂ |
| 10 | G | I | F | S | K | L | A | X¹ | K | K | I | X² | N | L | L | I | S | G | L | K | G | | | | 8 | mxy | —NH₂ |
| 11 | G | I | F | S | K | L | A | X¹ | K | K | I | X² | N | L | L | I | S | G | L | K | G | | | | 8 | pxy | —NH₂ |
| 12 | G | I | F | S | K | L | A | X¹ | K | K | I | X² | N | L | L | I | S | G | L | K | G | | | | 8 | but | —NH₂ |
| 13 | G | I | F | S | K | L | A | G | X¹ | K | I | K | X² | L | L | I | S | G | L | K | G | | | | 9 | pxy | —NH₂ |
| 14 | G | I | F | S | K | L | A | G | K | X¹ | I | K | N | X² | L | I | S | G | L | K | G | | | | 10 | mxy | —NH₂ |
| 15 | G | I | F | S | K | L | A | G | K | X¹ | I | K | N | X² | L | I | S | G | L | K | G | | | | 10 | pxy | —NH₂ |
| 16 | G | I | F | S | K | L | A | G | K | X¹ | I | K | N | X² | L | I | S | G | L | K | G | | | | 10 | but | —NH₂ |
| 17 | G | I | F | S | K | L | A | G | K | K | X¹ | K | N | L | X² | I | S | G | L | K | G | | | | 11 | mxy | —NH₂ |
| 18 | G | I | F | S | K | L | A | G | K | K | X¹ | K | N | L | X² | I | S | G | L | K | G | | | | 11 | pxy | —NH₂ |
| 19 | G | I | F | S | K | L | A | G | K | K | X¹ | K | N | L | X² | I | S | G | L | K | G | | | | 11 | but | —NH₂ |
| 20 | G | I | F | S | K | L | A | G | K | K | I | X¹ | N | L | L | X² | S | G | L | K | G | | | | 12 | mxy | —NH₂ |
| 21 | G | I | F | S | K | L | A | G | K | K | I | X¹ | N | L | L | X² | S | G | L | K | G | | | | 12 | pxy | —NH₂ |
| 22 | G | I | F | S | K | L | A | G | K | K | I | X¹ | N | L | L | X² | S | G | L | K | G | | | | 12 | but | —NH₂ |
| 23 | G | I | F | S | K | L | A | G | K | K | I | K | X¹ | L | L | I | X² | G | L | K | G | | | | 13 | mxy | —NH₂ |
| 24 | G | I | F | S | K | L | A | G | K | K | I | K | X¹ | L | L | I | X² | G | L | K | G | | | | 13 | pxy | —NH₂ |
| 25 | G | I | F | S | K | L | A | G | K | K | I | K | X¹ | L | L | I | X² | G | L | K | G | | | | 13 | but | —NH₂ |
| 26 | G | I | F | S | K | L | A | G | K | K | I | K | N | X¹ | L | I | S | X² | L | K | G | | | | 14 | mxy | —NH₂ |
| 27 | G | I | F | S | K | L | A | G | K | K | I | K | N | X¹ | L | I | S | X² | L | K | G | | | | 14 | pxy | —NH₂ |
| 28 | G | I | F | S | K | L | A | G | K | K | I | K | N | X¹ | L | I | S | X² | L | K | G | | | | 14 | but | —NH₂ |
| 29 | G | I | F | S | K | L | A | G | K | K | I | K | N | L | X¹ | I | S | G | X² | K | G | | | | 15 | mxy | —NH₂ |
| 30 | G | I | F | S | K | L | A | G | K | K | I | K | N | L | X¹ | I | S | G | X² | K | G | | | | 15 | pxy | —NH₂ |
| 31 | G | I | F | S | K | L | A | G | K | K | I | K | N | L | X¹ | I | S | G | X² | K | G | | | | 15 | but | —NH₂ |
| 32 | G | I | F | S | K | L | A | G | K | K | I | K | N | L | L | X¹ | S | G | L | X² | G | | | | 16 | mxy | —NH₂ |
| 33 | G | I | F | S | K | L | A | G | K | K | I | K | N | L | L | X¹ | S | G | L | X² | G | | | | 16 | pxy | —NH₂ |
| 34 | G | I | F | S | K | L | A | G | K | K | I | K | N | L | L | X¹ | S | G | L | X² | G | | | | 16 | but | —NH₂ |
| 35 | G | I | F | S | K | L | A | G | K | K | I | K | N | L | L | I | X¹ | G | L | K | X² | | | | 17 | but | —NH₂ |
| 36 | X¹ | I | F | S | X² | L | A | G | K | K | I | K | N | L | L | I | S | G | L | K | G | | | | 1 | mxy | —NH₂ |
| 37 | X¹ | I | F | S | X² | L | A | G | K | K | I | K | N | L | L | I | S | G | L | K | G | | | | 1 | pxy | —NH₂ |
| 38 | X¹ | I | F | S | X² | L | A | G | K | K | I | K | N | L | L | I | S | G | L | K | G | | | | 1 | but | —NH₂ |
| 39 | G | I | X¹ | S | K | L | X² | G | K | K | I | K | N | L | L | I | S | G | L | K | G | | | | 3 | mxy | —NH₂ |
| 40 | G | I | X¹ | S | K | L | X² | G | K | K | I | K | N | L | L | I | S | G | L | K | G | | | | 3 | pxy | —NH₂ |
| 41 | G | I | F | S | K | X¹ | A | G | K | X² | I | K | N | L | L | I | S | G | L | K | G | | | | 6 | mxy | —NH₂ |
| 42 | G | I | F | S | K | L | A | G | X¹ | K | I | K | X² | L | L | I | S | G | L | K | G | | | | 9 | mxy | —NH₂ |
| 43 | G | I | F | S | K | L | A | G | X¹ | K | I | K | X² | L | L | I | S | G | L | K | G | | | | 9 | but | —NH₂ |
| 44 | G | I | F | S | K | L | A | G | K | K | I | K | N | L | L | I | X¹ | G | L | K | X² | | | | 17 | mxy | —NH₂ |
| 45 | G | I | F | S | K | L | A | G | K | K | I | K | N | L | L | I | X¹ | G | L | K | X² | | | | 17 | pxy | —NH₂ |
| 46 | G | X¹ | F | S | K | X² | A | G | K | K | I | K | N | L | L | I | S | G | L | K | G | | | | 2 | mxy | —NH₂ |
| 47 | G | X¹ | F | S | K | X² | A | G | K | K | I | K | N | L | L | I | S | G | L | K | G | | | | 2 | pxy | —NH₂ |
| 48 | G | X¹ | F | S | K | X² | A | G | K | K | I | K | N | L | L | I | S | G | L | K | G | | | | 2 | but | —NH₂ |
| 91 | G | I | F | S | K | L | X¹ | G | K | K | X² | K | N | L | L | I | S | G | L | K | G | | | | 7 | mxy | —CO₂H |
| 92 | G | I | F | S | K | L | X¹ | G | K | K | X² | K | N | L | L | I | S | G | L | K | G | G | G | | 74 | mxy | —CO₂H |
| 93 | G | I | F | S | K | L | X¹ | G | K | K | X² | K | N | L | L | I | S | G | L | K | G | G | G | G | 65 | mxy | —CO₂H |
| 94 | G | I | F | S | K | L | X¹ | G | K | K | X² | K | N | L | L | I | S | G | L | K | G | G | G | K | 66 | mxy | —CO₂H |
| 95 | G | I | F | S | K | L | X¹ | G | K | K | X² | K | N | L | L | I | S | G | L | K | G | A | G | | 67 | mxy | —CO₂H |
| 96 | G | I | F | S | K | L | X¹ | G | K | K | X² | K | N | L | L | I | S | G | L | K | G | A | K | | 68 | mxy | —CO₂H |
| 97 | G | I | F | S | K | L | X¹ | A | K | K | X² | K | N | L | L | I | S | G | L | K | G | | | | 69 | mxy | —CO₂H |
| 98 | G | I | F | S | K | L | X¹ | G | K | K | X² | K | N | L | L | I | S | A | L | K | G | | | | 70 | mxy | —CO₂H |
| 99 | G | I | F | S | K | L | X¹ | A | K | K | X² | K | N | L | L | I | S | A | L | K | G | | | | 77 | mxy | —CO₂H |
| 100 | G | I | F | S | K | L | X¹ | G | K | K | X² | K | N | L | L | I | S | G | L | K | G | A | K | | 68 | but | —CO₂H |
| 101 | J | I | F | S | K | X² | A | G | K | X² | I | K | N | L | L | I | S | G | L | K | G | | | | 78 | but | —CO₂H |
| 102 | J | I | F | S | K | X² | A | G | K | X² | I | K | N | L | L | I | S | G | L | K | G | | | | 78 | but | —NH₂ |
| 103 | J | I | F | S | K | X² | A | G | K | X² | I | K | N | L | L | I | S | G | L | K | G | | | | 78 | but | —NHOH |

50. The peptide of paragraph 49, wherein the peptide is one of StAMPs 1-48 or 91-103, or a pharmaceutically acceptable salt thereof.
51. The peptide of any one of the preceding paragraphs, or a pharmaceutically acceptable salt thereof, wherein the peptide comprises one of the following peptides:

| | | | | | | | | | | | | | | | | | | | | | | SEQ | X¹-X² | |
|---|---|---|---|---|---|---|---|---|---|---|---|---|---|---|---|---|---|---|---|---|---|---|---|---|
| StAMP | | | | | | | | | | | | | | | | | | | | | | ID | Cross- | C- |
| # | Sequence | | | | | | | | | | | | | | | | | | | | | NO: | link | terminus |
| 49 | X¹ | I | F | S | K | L | A | X² | K | K | I | K | N | L | L | I | S | G | L | K | G | 18 | bbp | —NH₂ |
| 50 | X¹ | I | F | S | K | L | A | X² | K | K | I | K | N | L | L | I | S | G | L | K | G | 18 | bbn | —NH₂ |
| 51 | X¹ | I | F | S | K | L | A | X² | K | K | I | K | N | L | L | I | S | G | L | K | G | 18 | bbf | —NH₂ |
| 52 | G | X¹ | F | S | K | L | A | G | X² | K | I | K | N | L | L | I | S | G | L | K | G | 19 | bbn | —NH₂ |
| 53 | G | X¹ | F | S | K | L | A | G | X² | K | I | K | N | L | L | I | S | G | L | K | G | 19 | bbf | —NH₂ |
| 54 | G | I | X¹ | S | K | L | A | G | K | X² | I | K | N | L | L | I | S | G | L | K | G | 20 | bbp | —NH₂ |
| 55 | G | I | X¹ | S | K | L | A | G | K | X² | I | K | N | L | L | I | S | G | L | K | G | 20 | bbn | —NH₂ |
| 56 | G | I | X¹ | S | K | L | A | G | K | X² | I | K | N | L | L | I | S | G | L | K | G | 20 | bbf | —NH₂ |
| 57 | G | I | F | X¹ | K | L | A | G | K | K | X² | K | N | L | L | I | S | G | L | K | G | 21 | bbp | —NH₂ |
| 58 | G | I | F | X¹ | K | L | A | G | K | K | X² | K | N | L | L | I | S | G | L | K | G | 21 | bbn | —NH₂ |
| 59 | G | I | F | X¹ | K | L | A | G | K | K | X² | K | N | L | L | I | S | G | L | K | G | 21 | bbf | —NH₂ |
| 60 | G | I | F | S | X¹ | L | A | G | K | K | I | X² | N | L | L | I | S | G | L | K | G | 22 | bbp | —NH₂ |
| 61 | G | I | F | S | X¹ | L | A | G | K | K | I | X² | N | L | L | I | S | G | L | K | G | 22 | bbf | —NH₂ |
| 62 | G | I | F | S | X¹ | L | A | G | K | K | I | X² | N | L | L | I | S | G | L | K | G | 22 | bbn | —NH₂ |
| 63 | G | I | F | S | K | X¹ | A | G | K | K | I | K | X² | L | L | I | S | G | L | K | G | 23 | bbf | —NH₂ |
| 64 | G | I | F | S | K | X¹ | A | G | K | K | I | K | X² | L | L | I | S | G | L | K | G | 23 | bbn | —NH₂ |
| 65 | G | I | F | S | K | X¹ | A | G | K | K | I | K | X² | L | L | I | S | G | L | K | G | 23 | bbf | —NH₂ |
| 66 | G | I | F | S | K | L | A | X¹ | K | K | I | K | N | L | X² | I | S | G | L | K | G | 24 | bbp | —NH₂ |
| 67 | G | I | F | S | K | L | A | X¹ | K | K | I | K | N | L | X² | I | S | G | L | K | G | 24 | bbn | —NH₂ |
| 68 | G | I | F | S | K | L | A | X¹ | K | K | I | K | N | L | X² | I | S | G | L | K | G | 24 | bbf | —NH₂ |
| 69 | G | I | F | S | K | L | A | G | X¹ | K | I | K | N | L | L | X² | S | G | L | K | G | 25 | bbn | —NH₂ |
| 70 | G | I | F | S | K | L | A | G | X¹ | K | I | K | N | L | L | X² | S | G | L | K | G | 25 | bbf | —NH₂ |
| 71 | G | I | F | S | K | L | A | G | X¹ | K | I | K | N | L | L | X² | S | G | L | K | G | 25 | bbp | —NH₂ |
| 72 | G | I | F | S | K | L | A | G | K | X¹ | I | K | N | L | L | I | X² | G | L | K | G | 26 | bbp | —NH₂ |
| 73 | G | I | F | S | K | L | A | G | K | X¹ | I | K | N | L | L | I | X² | G | L | K | G | 26 | bbn | —NH₂ |
| 74 | G | I | F | S | K | L | A | G | K | X¹ | I | K | N | L | L | I | X² | G | L | K | G | 26 | bbf | —NH₂ |
| 75 | G | I | F | S | K | L | A | G | K | K | X¹ | K | N | L | L | I | S | X² | L | K | G | 27 | bbn | —NH₂ |
| 76 | G | I | F | S | K | L | A | G | K | K | X¹ | K | N | L | L | I | S | X² | L | K | G | 27 | bbf | —NH₂ |
| 77 | G | I | F | S | K | L | A | G | K | K | X¹ | K | N | L | L | I | S | X² | L | K | G | 27 | bbp | —NH₂ |
| 78 | G | I | F | S | K | L | A | G | K | K | I | X¹ | N | L | L | I | S | G | X² | K | G | 28 | bbn | —NH₂ |
| 79 | G | I | F | S | K | L | A | G | K | K | I | X¹ | N | L | L | I | S | G | X² | K | G | 28 | bbf | —NH₂ |
| 80 | G | I | F | S | K | L | A | G | K | K | I | C | N | L | L | I | S | G | C | K | G | 28 | bbp | —NH₂ |
| 81 | G | I | F | S | K | L | A | G | K | K | I | K | X¹ | L | L | I | S | G | L | X² | G | 29 | bbp | —NH₂ |
| 82 | G | I | F | S | K | L | A | G | K | K | I | K | X¹ | L | L | I | S | G | L | X² | G | 29 | bbn | —NH₂ |
| 83 | G | I | F | S | K | L | A | G | K | K | I | K | X¹ | L | L | I | S | G | L | X² | G | 29 | bbf | —NH₂ |
| 84 | G | I | F | S | K | L | A | G | K | K | I | K | N | X¹ | L | I | S | G | L | K | X² | 30 | bbp | —NH₂ |
| 85 | G | I | F | S | K | L | A | G | K | K | I | K | N | X¹ | L | I | S | G | L | K | X² | 30 | bbn | —NH₂ |
| 86 | G | I | F | S | K | L | A | G | K | K | I | K | N | X¹ | L | I | S | G | L | K | X² | 30 | bbf | —NH₂ |
| 87 | G | I | F | S | K | L | X¹ | G | K | K | I | K | N | X² | L | I | S | G | L | K | G | 31 | bbn | —NH₂ |
| 88 | G | I | F | S | K | L | X¹ | G | K | K | I | K | N | X² | L | I | S | G | L | K | G | 31 | bbf | —NH₂ |
| 89 | G | I | F | S | K | L | X¹ | G | K | K | I | K | N | X² | L | I | S | G | L | K | G | 31 | bbp | —NH₂ |

52. The peptide of paragraph 51, wherein the peptide is one of StAMPs 49-89, or a pharmaceutically acceptable salt thereof.
53. The peptide of any one of the preceding paragraphs, or a pharmaceutically acceptable salt thereof, wherein the peptide comprises on of the following peptides:

| | | | | | | | | | | | | | | | | | | | | | | SEQ | X¹-X² and | |
|---|---|---|---|---|---|---|---|---|---|---|---|---|---|---|---|---|---|---|---|---|---|---|---|---|
| StAMP | | | | | | | | | | | | | | | | | | | | | | ID | X³-X⁴ | C- |
| # | Sequence | | | | | | | | | | | | | | | | | | | | | NO: | Crosslink | terminus |
| 90 | G | I | F | S | K | L | X¹ | G | K | K | X² | K | N | L | L | I | X³ | G | L | K | X⁴ | 32 | pxy | —NH₂ |

54. The peptide of paragraph 53, wherein the peptide is StAMP 90, or a pharmaceutically acceptable salt thereof.
55. The peptide of any one of the preceding paragraphs, or a pharmaceutically acceptable salt thereof, wherein the reactive moieties are thiols.
56. The peptide of paragraph 55, or a pharmaceutically acceptable salt thereof, wherein X¹, X², X³, and X⁴ independently comprise α-sidechains of the following formula:

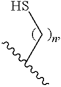

wherein each n is independently an integer from 1-10, inclusive.
57. The peptide of paragraph 55, or a pharmaceutically acceptable salt thereof, wherein each of X¹, X², X³, and X⁴ are cysteine (C).
58. A peptide, or a pharmaceutically acceptable salt thereof, comprising 10 or more contiguous amino acids of Esculentin-1A, wherein:
2 or 4 amino acids of the 10 or more contiguous amino acids of Esculentin are substituted by C;
optionally wherein the amino acid sequence includes 1 to 6 additional amino acid substitutions, inclusive.
   59. The peptide of paragraph 58, or a pharmaceutically acceptable salt thereof, comprising 20 or more contiguous amino acids of Esculentin-1A; wherein:
2 or 4 amino acids of the 20 or more contiguous amino acids of Esculentin are substituted by C;
optionally wherein the amino acid sequence includes 1 to 6 additional amino acid substitutions, inclusive.
   60. The peptide of paragraph 58 or 59, or a pharmaceutically acceptable salt thereof, wherein at least two C are separated by 3 amino acids.
   61. The peptide of paragraph 58 or 59, or a pharmaceutically acceptable salt thereof, wherein at least two C are separated by 6 amino acids.
   62. A peptide of any one of the preceding paragraphs, wherein the peptide comprises one of the following amino acid sequences:

(SEQ ID NO: 37)
C I F S C L A G K K I K N L L I S G L K G,
(SEQ ID NO: 38)
G C F S K C A G K K I K N L L I S G L K G,
(SEQ ID NO: 39)
G I C S K L C G K K I K N L L I S G L K G,
(SEQ ID NO: 40)
G I F C K L A C K K I K N L L I S G L K G,
(SEQ ID NO: 41)
G I F S C L A G C K I K N L L I S G L K G,
(SEQ ID NO: 42)
G I F S K C A G K C I K N L L I S G L K G,
(SEQ ID NO: 43)
G I F S K L C G K K C K N L L I S G L K G,
(SEQ ID NO: 82)
G I F S K L C G K K C K N L L I S G L K G G G,
(SEQ ID NO: 44)
G I F S K L A C K K I C N L L I S G L K G,
(SEQ ID NO: 45)
G I F S K L A G C K I K C L L I S G L K G,
(SEQ ID NO: 46)
G I F S K L A G K C I K N C L I S G L K G,
(SEQ ID NO: 47)
G I F S K L A G K K C K N L C I S G L K G,
(SEQ ID NO: 48)
G I F S K L A G K K I C N L L C S G L K G,
(SEQ ID NO: 49)
G I F S K L A G K K I K C L L I C G L K G,
(SEQ ID NO: 50)
G I F S K L A G K K I K N C L I S C L K G,
(SEQ ID NO: 51)
G I F S K L A G K K I K N L C I S G C K G,
(SEQ ID NO: 52)
G I F S K L A G K K I K N L L C S G L C G,
(SEQ ID NO: 53)
G I F S K L A G K K I K N L L I C G L K C,
(SEQ ID NO: 79)
G I F S K L C G K K C K N L L I S G L K G G G G,
(SEQ ID NO: 80)
G I F S K L C G K K C K N L L I S G L K G G G K,
(SEQ ID NO: 81)
G I F S K L C G K K C K N L L I S G L K G A G,
(SEQ ID NO: 83)
G I F S K L C G K K C K N L L I S G L K G A K,
(SEQ ID NO: 84)
G I F S K L C A K K C K N L L I S G L K G,
(SEQ ID NO: 85)
G I F S K L C G K K C K N L L I S A L K G,
(SEQ ID NO: 86)
G I F S K L C A K K C K N L L I S A L K G,
(SEQ ID NO: 87)
JIFSKCAGKCIKNLLISGLKG,

or a pharmaceutically acceptable salt thereof.
63. The peptide of paragraph 63, wherein the peptide is of:
(i) one of SEQ ID NOs: 37-53, or a pharmaceutically acceptable salt thereof, wherein the C-terminus is amidated with —NH₂;
(ii) SEQ ID NO: 82, or a pharmaceutically acceptable salt thereof, wherein the C-terminus is —CO₂H;
(iii) of any one of SEQ ID NO: 79-81 and 83-86, wherein the wherein the C-terminus is —CO₂H; or
(iv) SEQ ID NO: 87, or a pharmaceutically acceptable salt thereof, wherein the C-terminus is —CO₂H or amidated with —NH₂ or —NHOH.
   64. The peptide of any one of the preceding paragraphs, wherein the peptide comprises one of the following amino acid sequences:

(SEQ ID NO: 54)
C I F S K L A C K K I K N L L I S G L K G,
(SEQ ID NO: 55)
G C F S K L A G C K I K N L L I S G L K G,
(SEQ ID NO: 56)
G I C S K L A G K C I K N L L I S G L K G,
(SEQ ID NO: 57)
G I F C K L A G K K C K N L L I S G L K G,
(SEQ ID NO: 58)
G I F S C L A G K K I C N L L I S G L K G,
(SEQ ID NO: 59)
G I F S K C A G K K I K C L L I S G L K G,
(SEQ ID NO: 60)
G I F S K L A C K K I K N L C I S G L K G,
(SEQ ID NO: 61)
G I F S K L A G C K I K N L L C S G L K G,
(SEQ ID NO: 62)
G I F S K L A G K C I K N L L I C G L K G,
(SEQ ID NO: 63)
G I F S K L A G K K C K N L L I S C L K G,
(SEQ ID NO: 64)
G I F S K L A G K K I C N L L I S G C K G,
(SEQ ID NO: 65)
G I F S K L A G K K I K C L L I S G L C G,
(SEQ ID NO: 66)
G I F S K L A G K K I K N C L I S G L K C,
(SEQ ID NO: 67)
G I F S K L C G K K I K N C L I S G L K G,

or a pharmaceutically acceptable salt thereof.
65. The peptide of paragraph 64, wherein the peptide is one of SEQ ID NOs: 54-67, or a pharmaceutically acceptable salt thereof; wherein the C-terminus is amidated with —NH₂.
66. The peptide of any one of the preceding paragraphs, wherein the peptide comprises the following amino acid sequence:

(SEQ ID NO: 68)
G I F S K L C G K K C K N L L I C G L K C,

or a pharmaceutically acceptable salt thereof.
67. The peptide of paragraph 66, wherein the peptide is SEQ ID NO: 68, or a pharmaceutically acceptable salt thereof; wherein the C-terminus is amidated with —NH₂.
68. A pharmaceutical composition comprising a peptide of any one of paragraphs 1-54, or a pharmaceutically acceptable salt thereof, and a pharmaceutically acceptable excipient.
69. A method of treating an infectious disease in a subject comprising administering to the subject a peptide of any one of paragraphs 1-54, or a pharmaceutically acceptable salt thereof, or a pharmaceutical composition thereof.
70. The method of paragraph 69, wherein the infectious disease is a bacterial infection, viral infection, protozoal infection, or fungal infection.
71. A method of treating a bacterial infection in a subject comprising administering to the subject a peptide of any one of paragraphs 1-54, or a pharmaceutically acceptable salt thereof, or a pharmaceutical composition thereof.
72. The method of paragraph 71, wherein the bacterial infection is a Gram-negative bacterial infection.
73. The method of paragraph 71 or 72, wherein the bacterial infection is an antibiotic-resistant bacterial infection.
74. The method of any one of paragraphs 71-73, wherein the bacterial infection is a Gram-negative, antibiotic-resistant bacterial infection.
75. The method of paragraph 73 or 74, wherein the antibiotic-resistant bacterial infection is caused by a bacteria resistant to one or more antibiotics selected from the group consisting of polymyxins, aminoglycosides, cephalosporins, penicillins, fluoroquinolones, tetracyclines, and β-lactams, and/or one or more antibiotics selected from the group consisting of carbapenem, vancomycin, methicillin, clarithromycin, ampicillin, ceftazidime, ceftriaxone, ciprofloxacin, gentamicin, and tobramycin.
76. The method of any one of paragraphs 71-75, wherein the bacterial infection is caused by E. coli, A. baumannii, P. aeruginosa, K. pneumoniae, S. maltophilia, or B. cepacia.
77. The method of any one of paragraphs 69-76, wherein the peptide has reduced renal toxicity as compared to Esculentin-1A or a fragment thereof.
78. The method of any one of paragraphs 69-76, wherein the peptide has reduced renal toxicity as compared to pexiganan.
79. The method of any one of paragraphs 69-76, wherein the peptide has reduced hepatic toxicity as compared to Esculentin-1A or a fragment thereof.
80. The method of any one of paragraphs 69-76, wherein the peptide has reduced hepatic toxicity as compared to pexiganan.
81. The method of any one of paragraphs 69-76, wherein the peptide has reduced hemolytic activity as compared to a corresponding peptide comprising SEQ ID NO: 1 or 2.
82. The method of any one of paragraphs 69-76, wherein the peptide has reduced hemolytic activity as compared to melittin.
83. The method of any one of paragraphs 69-82, wherein the peptide, pharmaceutically acceptable salt thereof, or pharmaceutical composition thereof is administered intravenously.
84. The method of any one of paragraphs 69-82, wherein the peptide, pharmaceutically acceptable salt thereof, or pharmaceutical composition thereof is administered via inhalation.
85. The method of any one of paragraphs 69-84, wherein the subject is a human.
86. A method of killing and/or inhibiting the growth of bacteria comprising contacting the bacteria with a peptide of any one of paragraphs 1-54, or a pharmaceutically acceptable salt thereof, or a pharmaceutical composition thereof.
87. The method of paragraph 86, wherein the bacteria is E. coli, A. baumannii, P. aeruginosa, K. pneumoniae, S. maltophilia, or B. cepacia.
88. A method of selectively killing and/or inhibiting the growth of microbial cells over mammalian cells comprising contacting the microbial and mammalian cells with a peptide of any one of paragraphs 1-54, or a pharmaceutically acceptable salt thereof, or a pharmaceutical composition thereof.
89. A method of selectively lysing microbial cells over mammalian cells comprising contacting the microbial and mammalian cells with a peptide of any one of paragraphs 1-54, or a pharmaceutically acceptable salt thereof, or a pharmaceutical composition thereof.
90. The method of paragraph 88 or 89, wherein the microbial cells are bacterial cells.
91. The method of paragraph 90, wherein the bacterial cells are E. coli cells, A. baumannii cells, P. aeruginosa cells, K. pneumoniae, S. maltophilia, or B. cepacia cells.
92. A peptide of any one of paragraphs 1-54, or a pharmaceutically acceptable salt thereof, or a pharmaceutical composition thereof, for use in a method of any one of paragraphs 69-91.
93. Use of a peptide of any one of paragraphs 1-54, or a pharmaceutically acceptable salt thereof, or a pharmaceutical composition thereof, for the preparation of a medicament.
94. A kit comprising a peptide of any one of paragraphs 1-54, or a pharmaceutically acceptable salt thereof, or a pharmaceutical composition thereof; and optionally instructions for use.
95. A method of preparing a crosslinked peptide of any one of the preceding paragraphs comprising a step of reacting an uncrosslinked peptide of any one of the preceding paragraphs under conditions sufficient to form the crosslinks connecting X¹ and X², and X³ and X⁴.
96. The method of paragraph 95, wherein the step of reacting involves a contacting the uncrosslinked polypeptide with a dihalide crosslinking reagent.

### EQUIVALENTS AND SCOPE

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein.

It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A peptide, or a pharmaceutically acceptable salt thereof, comprising 10 or more contiguous amino acids of Esculentin-1A and comprising exactly one internal crosslink connecting two crosslinked amino acids, wherein:
two amino acids of the 10 or more contiguous amino acids of Esculentin-1A are independently substituted by X¹ and X²; and
X¹ and X² are independently the crosslinked amino acids;
optionally wherein the amino acid sequence includes 1 to 3 amino acid substitutions, inclusive, at positions other than X¹ and X².

2. A peptide, or a pharmaceutically acceptable salt thereof, comprising 10 or more contiguous amino acids of Esculentin-1A; wherein:
exactly two amino acids of the 10 or more contiguous amino acids of Esculentin-1A are independently substituted by X¹ and X²; and
X¹ and X² are each independently amino acids comprising reactive moieties capable of forming a crosslink with the other;
optionally wherein the amino acid sequence includes 1 to 3 amino acid substitutions, inclusive, at positions other than X¹ and X².

3. The peptide of claim 1 or 2, or a pharmaceutically acceptable salt thereof, comprising 20 or more contiguous amino acids of Esculentin-1A; optionally wherein the amino acid sequence includes 1 to 6 amino acid substitutions, inclusive, at positions other than X¹ and X².

4. The peptide of any one of claims 1-3, wherein X¹ and X² are separated by 3 amino acids, forming an i+4 crosslink.

5. The peptide of any one of claims 1-3, wherein X¹ and X² are separated by 6 amino acids, forming an i+7 crosslink.

6. The peptide of claim 1, wherein the peptide comprises the following amino acid sequence:
(SEQ ID NO: 7)
G I F S K L X¹ G K K X² K N L L I S G L K G,
or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².

7. The peptide of claim 6, wherein the peptide comprises the following amino acid sequence:
(SEQ ID NO: 74)
G I F S K L X¹ G K K X² K N L L I S G L K G G G,
or a pharmaceutically acceptable salt thereof.

8. The peptide of claim 6, wherein the peptide comprises one of the following amino acid sequences:
(SEQ ID NO: 65)
G I F S K L X¹ G K K X² K N L L I S G L K G G G G,
(SEQ ID NO: 66)
G I F S K L X¹ G K K X² K N L L I S G L K G G G K,
(SEQ ID NO: 67)
G I F S K L X¹ G K K X² K N L L I S G L K G A G,
(SEQ ID NO: 68)
G I F S K L X¹ G K K X² K N L L I S G L K G A K,
(SEQ ID NO: 69)
G I F S K L X¹ A K K X² K N L L I S G L K G,
(SEQ ID NO: 70)
G I F S K L X¹ G K K X² K N L L I S A L K G,
(SEQ ID NO: 77)
G I F S K L X¹ A K K X² K N L L I S A L K G,
or a pharmaceutically acceptable salt thereof.

9. The peptide of claim 1, wherein the peptide comprises one of the following amino acid sequences:
(SEQ ID NO: 1)
X¹ I F S X² L A G K K I K N L L I S G L K G,
(SEQ ID NO: 2)
G X¹ F S K X² A G K K I K N L L I S G L K G,
(SEQ ID NO: 3)
G I X¹ S K L X² G K K I K N L L I S G L K G,
(SEQ ID NO: 4)
G I F X¹ K L A X² K K I K N L L I S G L K G,
(SEQ ID NO: 5)
G I F S X¹ L A G X² K I K N L L I S G L K G,
(SEQ ID NO: 6)
G I F S K X¹ A G K X² I K N L L I S G L K G,
(SEQ ID NO: 7)
G I F S K L X¹ G K K X² K N L L I S G L K G,
(SEQ ID NO: 8)
G I F S K L A X¹ K K I X² N L L I S G L K G,
(SEQ ID NO: 9)
G I F S K L A G X¹ K I K X² L L I S G L K G,
(SEQ ID NO: 10)
G I F S K L A G K X¹ I K N X² L I S G L K G,
(SEQ ID NO: 11)
G I F S K L A G K K X¹ K N L X² I S G L K G,
(SEQ ID NO: 12)
G I F S K L A G K K I X¹ N L L X² S G L K G,
(SEQ ID NO: 13)
G I F S K L A G K K I K X¹ L L I X² G L K G,
(SEQ ID NO: 14)
G I F S K L A G K K I K N X¹ L I S X² L K G,
(SEQ ID NO: 15)
G I F S K L A G K K I K N L X¹ I S G X² K G,
(SEQ ID NO: 16)
G I F S K L A G K K I K N L L X¹ S G L X² G,
(SEQ ID NO: 17)
G I F S K L A G K K I K N L L I X¹ G L K X²,
or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².

10. The peptide of claim 9, wherein the peptide comprises the amino acid sequence:
JIFSKXIAGKX²IKNLLISGLKG (SEQ ID NO: 78), or a pharmaceutically acceptable salt thereof.

11. The peptide of claim 1, wherein the peptide comprises one of the following amino acid sequences:
(SEQ ID NO: 18)
X¹ I F S K L A X² K K I K N L L I S G L K G,
(SEQ ID NO: 19)
G X¹ F S K L A G X² K I K N L L I S G L K G,
(SEQ ID NO: 20)
G I X¹ S K L A G K X² I K N L L I S G L K G,
(SEQ ID NO: 21)
G I F X¹ K L A G K K X² K N L L I S G L K G,
(SEQ ID NO: 22)
G I F S X¹ L A G K K I X² N L L I S G L K G,
(SEQ ID NO: 23)
G I F S K X¹ A G K K I K X² L L I S G L K G,
(SEQ ID NO: 24)
G I F S K L A X¹ K K I K N L X² I S G L K G,
(SEQ ID NO: 25)
G I F S K L A G X¹ K I K N L L X² S G L K G,
(SEQ ID NO: 26)
G I F S K L A G K X¹ I K N L L I X² G L K G,
(SEQ ID NO: 27)
G I F S K L A G K K X¹ K N L L I S X2 L K G,
(SEQ ID NO: 28)
G I F S K L A G K K I X¹ N L L I S G X² K G,
(SEQ ID NO: 29)
G I F S K L A G K K I K X¹ L L I S G L X² G,
(SEQ ID NO: 30)
G I F S K L A G K K I K N X¹ L I S G L K X²,
(SEQ ID NO: 31)
G I F S K L X¹ G K K I K N X² L I S G L K G,
or a pharmaceutically acceptable salt thereof, wherein:
X¹ and X² are independently amino acids connected via a crosslink;
optionally wherein the amino acid sequence includes 1 to 6 amino acid substitutions, inclusive, at positions other than X¹ and X².

12. The peptide of claim 2, wherein the peptide comprises any one of SEQ ID NOs: 1-31, 65-70, 74, and 77;
optionally wherein the amino acid sequence includes 1 to 6 amino acid substitutions, inclusive, at positions other than X¹ and X².

13. The peptide of any one of claims 1-12, or a pharmaceutically acceptable salt thereof, wherein the amino acid sequence includes 1 to 3 amino acid substitutions, inclusive, at positions other than X¹ and X².

14. The peptide of any one of claims 1-12, or a pharmaceutically acceptable salt thereof, wherein the amino acid sequence includes 1 amino acid substitutions at a position other than X¹ or X².

15. The peptide of any one of claims 1-12, or a pharmaceutically acceptable salt thereof, wherein the amino acid sequence does not include an amino acid substitution.

16. A peptide comprising the amino acid sequence:
(SEQ ID NO: 32)
G I F S KL X¹ G K K X² K N L L I X³ G L K X⁴,
or a pharmaceutically acceptable salt thereof, wherein:
X¹, X², X³, and X⁴ are independently amino acids;
X¹ and X² are connected via a crosslink, and X³ and X⁴ are connected via a crosslink; and
optionally wherein the amino acid sequence includes 1 to 6 amino acid substitutions, inclusive, at positions other than X¹, X², X³, and X⁴.

17. A peptide comprising the amino acid sequence SEQ ID NO: 32, or a pharmaceutically acceptable salt thereof, wherein:
X¹, X², X³, and X⁴ are independently amino acids;
X¹ and X² each independently comprise a reactive moiety capable of forming a crosslink with the other, and X³ and X⁴ each independently comprise a reactive moiety capable of forming a crosslink with the other; and
the amino acid sequence includes 1 to 6 amino acid substitutions, inclusive, at positions other than X¹, X², X³, and X⁴.

18. The peptide of any one of claims 1-17, or a pharmaceutically acceptable salt thereof, wherein one or more instances of K are independently substituted by an amino acid selected from Orn, Dab, Dap, R, and hArg.

19. The peptide of claim 18, or a pharmaceutically acceptable salt thereof, wherein each instance of K is independently substituted by an amino acid selected from Orn, Dab, Dap, R, and hArg.

20. The peptide of any one of claims 1-19, wherein one or more instances of F are independently substituted by an amino acid selected from F¹, F⁵, F⁴, F², and F³.

21. The peptide of claim 20, or a pharmaceutically acceptable salt thereof, wherein each instance of F is independently substituted by an amino acid selected from F¹, F⁵, F⁴, F², and F³.

22. The peptide of any one of claims 1-21, or a pharmaceutically acceptable salt thereof, further comprising a small molecule, lipophilic group, or polymer conjugated to the C-terminus of the peptide.

23. The peptide of claim 22, wherein the lipophilic group is a lipid or fatty acid.

24. The peptide of claim 22, wherein the peptide comprises PEG conjugated to C-terminus, or wherein the peptide is amidated at the C-terminus with a group of the formula:
—NH(CH₂CH₂O)₁₋₂₀CH₂CH₂CONH₂.

25. The peptide of any one of claims 1-22, or a pharmaceutically acceptable salt thereof, comprising an amino acid or peptide conjugated to the C-terminus of the peptide.

26. The peptide of claim 25, or a pharmaceutically acceptable salt thereof, comprising one of the following amino acid sequences conjugated to the C-terminus of the peptide:
GE,
AG,
AA,
AK,
GG,
GGE,
GGS,
GGG,
GGK,
GGQ,
(SEQ ID NO: 88)
GGGE,
(SEQ ID NO: 89)
GGEE,
or
(SEQ ID NO: 90)
GGSGGS.

27. The peptide of claim 22, or a pharmaceutically acceptable salt thereof, comprising a polymyxin conjugated to the C-terminus of the peptide.

28. The peptide of any one of claims 1-21, wherein the C-terminus is amidated.

29. The peptide of any one of claims 1-28, wherein the C-terminus is amidated with —NH₂.

30. The peptide of any one of claims 1-28, wherein the C-terminus is amidated with —NHOH.

31. The peptide of any one of claims 1-30, or a pharmaceutically acceptable salt thereof, wherein the peptide is 100 amino acids or fewer in length.

32. The peptide of any one of claims 1-31, or a pharmaceutically acceptable salt thereof, wherein the peptide is 30 amino acids or fewer in length.

33. The peptide of any one of the preceding claims, or a pharmaceutically acceptable salt thereof, wherein the crosslinks are attached to the α-positions of the amino acids X¹, X², X³, and X⁴.

34. The peptide of any one of the preceding claims, or a pharmaceutically acceptable salt thereof, wherein each crosslink is independently optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, optionally substituted acylene, or any combination thereof.

35. The peptide of any one of the preceding claims, or a pharmaceutically acceptable salt thereof, wherein each crosslink is independently a dithio crosslink.

36. A peptide, or a pharmaceutically acceptable salt thereof, comprising 10 or more contiguous amino acids of Esculentin-1A and at least one internal dithio crosslink, wherein:
at least 2 amino acids of the 10 or more contiguous amino acids of Esculentin-1A are substituted by a pair of dithio-crosslinked amino acids;
optionally wherein the amino acid sequence includes 1 to 3 additional amino acid substitutions, inclusive.

37. The peptide of claim 29, or a pharmaceutically acceptable salt thereof, comprising 20 or more contiguous amino acids of Esculentin-1A; wherein:
at least 2 amino acids of the 20 or more contiguous amino acids of Esculentin-1A are substituted by a pair dithio-crosslinked amino acids;
optionally wherein the amino acid sequence includes 1 to 6 additional amino acid substitutions, inclusive.

38. The peptide of claim 36 or 37, or a pharmaceutically acceptable salt thereof, wherein the pair of dithio-crosslinked amino acids are separated by 3 amino acids, forming an i+4 dithio crosslink.

39. The peptide of claim 36 or 37, or a pharmaceutically acceptable salt thereof, wherein the pair of dithio-crosslinked amino acids are separated by 6 amino acids, forming an i+7 dithio crosslink.

40. The peptide of any of claims 35-39, or a pharmaceutically acceptable salt thereof, wherein each dithio crosslink is independently of the following formula: 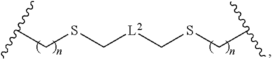
wherein each n is independently an integer from 1-10, inclusive;
L² is optionally substituted alkylene, 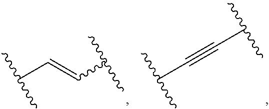
optionally substituted arylene, optionally substituted heteroarylene, or -A¹-A¹-; wherein each instance of A¹ is independently optionally substituted arylene or optionally substituted heteroarylene.

41. The peptide of claim 40, or a pharmaceutically acceptable salt thereof, wherein each dithio crosslink is independently of one of the following formulae:

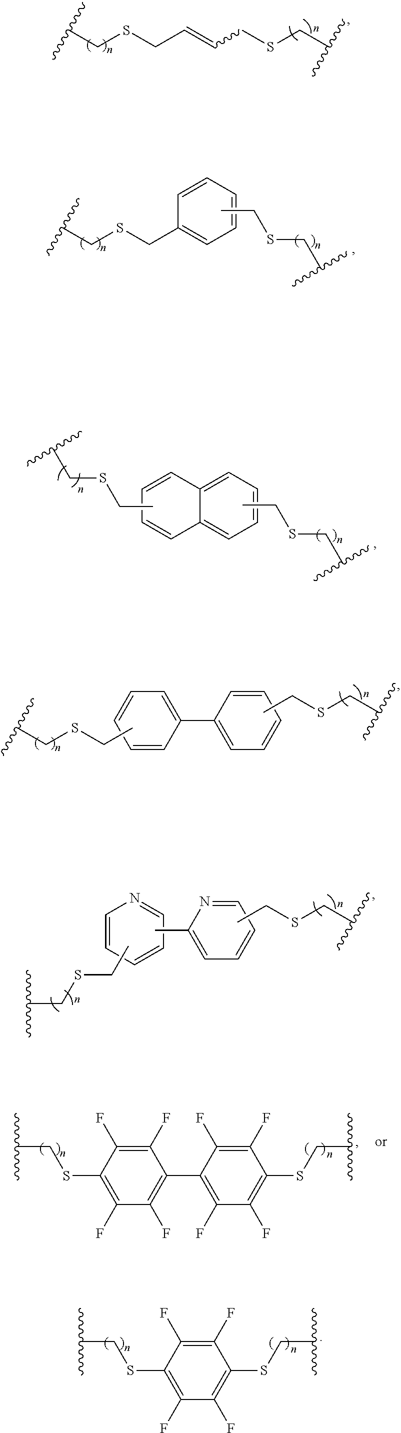

42. The peptide of claim 40 or 41, or a pharmaceutically acceptable salt thereof, wherein each instance of n is 1.

43. The peptide of any one of the preceding claims, or a pharmaceutically acceptable salt thereof, wherein each crosslink is independently of one of the following formulae:

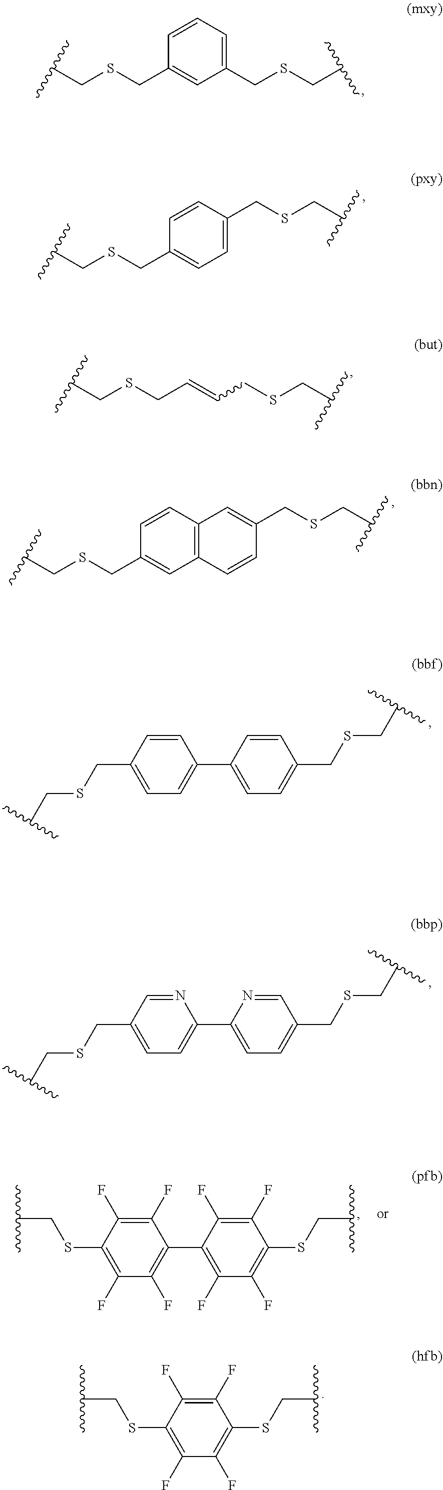

44. The peptide of any one of the preceding claims, or a pharmaceutically acceptable salt thereof, wherein each crosslink is independently about 5 Å to about 35 Å in length, inclusive.

45. The peptide of any one of the preceding claims, or a pharmaceutically acceptable salt thereof, wherein the length of each crosslink is approximately equal to the length of 5 to 25 carbon-carbon and/or carbon-sulfur bonds, inclusive.

46. The peptide of any one of the preceding claims, or a pharmaceutically acceptable salt thereof, wherein at least one crosslink spans an α-helix of the peptide.

47. The peptide of any one of the preceding claims, or a pharmaceutically acceptable salt thereof, wherein at least one crosslink stabilizes an α-helix of the peptide.

48. The peptide of any one of the preceding claims, or a pharmaceutically acceptable salt thereof, wherein the peptide has increased α-helicity as compared to a corresponding uncrosslinked peptide.

49. The peptide of any one of the preceding claims, or a pharmaceutically acceptable salt thereof, wherein the peptide comprises one of the following peptides:

| StAMP | | | | | | | | | | | | | | | | | | | | | | | | | SEQ ID | X¹-X² | C- |
|---|---|---|---|---|---|---|---|---|---|---|---|---|---|---|---|---|---|---|---|---|---|---|---|---|---|---|---|
| # | Sequence | | | | | | | | | | | | | | | | | | | | | | | | NO: | Crosslink | terminus |
| 1 | G | I | X¹ | S | K | L | X² | G | K | K | I | K | N | L | L | I | S | G | L | K | G | | | | 3 | but | —NH₂ |
| 2 | G | I | F | S | K | L | X¹ | G | K | K | X² | K | N | L | L | I | S | G | L | K | G | | | | 7 | mxy | —NH₂ |
| 3 | G | I | F | S | K | L | X¹ | G | K | K | X² | K | N | L | L | I | S | G | L | K | G | | | | 7 | pxy | —NH₂ |
| 4 | G | I | F | S | K | L | X¹ | G | K | K | X² | K | N | L | L | I | S | G | L | K | G | | | | 7 | but | —NH₂ |
| 5 | G | I | F | X¹ | K | L | A | X² | K | K | I | K | N | L | L | I | S | G | L | K | G | | | | 4 | mxy | —NH₂ |
| 6 | G | I | F | X¹ | K | L | A | X² | K | K | I | K | N | L | L | I | S | G | L | K | G | | | | 4 | pxy | —NH₂ |
| 7 | G | I | F | X¹ | K | L | A | X² | K | K | I | K | N | L | L | I | S | G | L | K | G | | | | 4 | but | —NH₂ |
| 8 | G | I | F | S | K | X¹ | A | G | K | X² | I | K | N | L | L | I | S | G | L | K | G | | | | 6 | pxy | —NH₂ |
| 9 | G | I | F | S | K | X¹ | A | G | K | X² | I | K | N | L | L | I | S | G | L | K | G | | | | 6 | but | —NH₂ |
| 10 | G | I | F | S | K | L | A | X¹ | K | K | I | X² | N | L | L | I | S | G | L | K | G | | | | 8 | mxy | —NH₂ |
| 11 | G | I | F | S | K | L | A | X¹ | K | K | I | X² | N | L | L | I | S | G | L | K | G | | | | 8 | pxy | —NH₂ |
| 12 | G | I | F | S | K | L | A | X¹ | K | K | I | X² | N | L | L | I | S | G | L | K | G | | | | 8 | but | —NH₂ |
| 13 | G | I | F | S | K | L | A | G | X¹ | K | I | K | X² | L | L | I | S | G | L | K | G | | | | 9 | pxy | —NH₂ |
| 14 | G | I | F | S | K | L | A | G | K | X¹ | I | K | N | X² | L | I | S | G | L | K | G | | | | 10 | mxy | —NH₂ |
| 15 | G | I | F | S | K | L | A | G | K | X¹ | I | K | N | X² | L | I | S | G | L | K | G | | | | 10 | pxy | —NH₂ |
| 16 | G | I | F | S | K | L | A | G | K | X¹ | I | K | N | X² | L | I | S | G | L | K | G | | | | 10 | but | —NH₂ |
| 17 | G | I | F | S | K | L | A | G | K | K | X¹ | K | N | L | X² | I | S | G | L | K | G | | | | 11 | mxy | —NH₂ |
| 18 | G | I | F | S | K | L | A | G | K | K | X¹ | K | N | L | X² | I | S | G | L | K | G | | | | 11 | pxy | —NH₂ |
| 19 | G | I | F | S | K | L | A | G | K | K | X¹ | K | N | L | X² | I | S | G | L | K | G | | | | 11 | but | —NH₂ |
| 20 | G | I | F | S | K | L | A | G | K | K | I | X¹ | N | L | L | X² | S | G | L | K | G | | | | 12 | mxy | —NH₂ |
| 21 | G | I | F | S | K | L | A | G | K | K | I | X¹ | N | L | L | X² | S | G | L | K | G | | | | 12 | pxy | —NH₂ |
| 22 | G | I | F | S | K | L | A | G | K | K | I | X¹ | N | L | L | X² | S | G | L | K | G | | | | 12 | but | —NH₂ |
| 23 | G | I | F | S | K | L | A | G | K | K | I | K | X¹ | L | L | I | X² | G | L | K | G | | | | 13 | mxy | —NH₂ |
| 24 | G | I | F | S | K | L | A | G | K | K | I | K | X¹ | L | L | I | X² | G | L | K | G | | | | 13 | pxy | —NH₂ |
| 25 | G | I | F | S | K | L | A | G | K | K | I | K | X¹ | L | L | I | X² | G | L | K | G | | | | 13 | but | —NH₂ |
| 26 | G | I | F | S | K | L | A | G | K | K | I | K | N | X¹ | L | I | S | X² | L | K | G | | | | 14 | mxy | —NH₂ |
| 27 | G | I | F | S | K | L | A | G | K | K | I | K | N | X¹ | L | I | S | X² | L | K | G | | | | 14 | pxy | —NH₂ |
| 28 | G | I | F | S | K | L | A | G | K | K | I | K | N | X¹ | L | I | S | X² | L | K | G | | | | 14 | but | —NH₂ |
| 29 | G | I | F | S | K | L | A | G | K | K | I | K | N | L | X¹ | I | S | G | X² | K | G | | | | 15 | mxy | —NH₂ |
| 30 | G | I | F | S | K | L | A | G | K | K | I | K | N | L | X¹ | I | S | G | X² | K | G | | | | 15 | pxy | —NH₂ |
| 31 | G | I | F | S | K | L | A | G | K | K | I | K | N | L | X¹ | I | S | G | X² | K | G | | | | 15 | but | —NH₂ |
| 32 | G | I | F | S | K | L | A | G | K | K | I | K | N | L | L | X¹ | S | G | L | X² | G | | | | 16 | mxy | —NH₂ |
| 33 | G | I | F | S | K | L | A | G | K | K | I | K | N | L | L | X¹ | S | G | L | X² | G | | | | 16 | pxy | —NH₂ |
| 34 | G | I | F | S | K | L | A | G | K | K | I | K | N | L | L | X¹ | S | G | L | X² | G | | | | 16 | but | —NH₂ |
| 35 | G | I | F | S | K | L | A | G | K | K | I | K | N | L | L | I | X¹ | G | L | K | X² | | | | 17 | but | —NH₂ |
| 36 | X¹ | I | F | S | X² | L | A | G | K | K | I | K | N | L | L | I | S | G | L | K | G | | | | 1 | mxy | —NH₂ |
| 37 | X¹ | I | F | S | X² | L | A | G | K | K | I | K | N | L | L | I | S | G | L | K | G | | | | 1 | pxy | —NH₂ |
| 38 | X¹ | I | F | S | X² | L | A | G | K | K | I | K | N | L | L | I | S | G | L | K | G | | | | 1 | but | —NH₂ |
| 39 | G | I | X¹ | S | K | L | X² | G | K | K | I | K | N | L | L | I | S | G | L | K | G | | | | 3 | mxy | —NH₂ |
| 40 | G | I | X¹ | S | K | L | X² | G | K | K | I | K | N | L | L | I | S | G | L | K | G | | | | 3 | pxy | —NH₂ |
| 41 | G | I | F | S | K | X¹ | A | G | K | X² | I | K | N | L | L | I | S | G | L | K | G | | | | 6 | mxy | —NH₂ |
| 42 | G | I | F | S | K | L | A | G | X¹ | K | I | K | X² | L | L | I | S | G | L | K | G | | | | 9 | mxy | —NH₂ |
| 43 | G | I | F | S | K | L | A | G | X¹ | K | I | K | X² | L | L | I | S | G | L | K | G | | | | 9 | but | —NH₂ |
| 44 | G | I | F | S | K | L | A | G | K | K | I | K | N | L | L | I | X¹ | G | L | K | X² | | | | 17 | mxy | —NH₂ |
| 45 | G | I | F | S | K | L | A | G | K | K | I | K | N | L | L | I | X¹ | G | L | K | X² | | | | 17 | pxy | —NH₂ |
| 46 | G | X¹ | F | S | K | X² | A | G | K | K | I | K | N | L | L | I | S | G | L | K | G | | | | 2 | mxy | —NH₂ |
| 47 | G | X¹ | F | S | K | X² | A | G | K | K | I | K | N | L | L | I | S | G | L | K | G | | | | 2 | pxy | —NH₂ |
| 48 | G | X¹ | F | S | K | X² | A | G | K | K | I | K | N | L | L | I | S | G | L | K | G | | | | 2 | but | —NH₂ |
| 91 | G | I | F | S | K | L | X¹ | G | K | K | X² | K | N | L | L | I | S | G | L | K | G | | | | 7 | mxy | —CO₂H |
| 92 | G | I | F | S | K | L | X¹ | G | K | K | X² | K | N | L | L | I | S | G | L | K | G | G | G | | 74 | mxy | —CO₂H |
| 93 | G | I | F | S | K | L | X¹ | G | K | K | X² | K | N | L | L | I | S | G | L | K | G | G | G | G | 65 | mxy | —CO₂H |
| 94 | G | I | F | S | K | L | X¹ | G | K | K | X² | K | N | L | L | I | S | G | L | K | G | G | G | K | 66 | mxy | —CO₂H |
| 95 | G | I | F | S | K | L | X¹ | G | K | K | X² | K | N | L | L | I | S | G | L | K | G | A | G | | 67 | mxy | —CO₂H |
| 96 | G | I | F | S | K | L | X¹ | G | K | K | X² | K | N | L | L | I | S | G | L | K | G | A | K | | 68 | mxy | —CO₂H |
| 97 | G | I | F | S | K | L | X¹ | A | K | K | X² | K | N | L | L | I | S | G | L | K | G | | | | 69 | mxy | —CO₂H |
| 98 | G | I | F | S | K | L | X¹ | G | K | K | X² | K | N | L | L | I | S | A | L | K | G | | | | 70 | mxy | —CO₂H |
| 99 | G | I | F | S | K | L | X¹ | A | K | K | X² | K | N | L | L | I | S | G | L | K | G | | | | 77 | mxy | —CO₂H |
| 100 | G | I | F | S | K | L | X¹ | G | K | K | X² | K | N | L | L | I | S | G | L | K | G | A | K | | 68 | but | —CO₂H |
| 101 | J | I | F | S | K | X² | A | G | K | K | I | K | N | L | L | I | S | G | L | K | G | | | | 78 | but | —CO₂H |
| 102 | J | I | F | S | K | X² | A | G | K | K | I | K | N | L | L | I | S | G | L | K | G | | | | 78 | but | —NH₂ |
| 103 | J | I | F | S | K | X² | A | G | K | K | I | K | N | L | L | I | S | G | L | K | G | | | | 78 | but | —NHOH |

50. The peptide of claim 49, wherein the peptide is one of StAMPs 1-48 or 91-103, or a pharmaceutically acceptable salt thereof.

51. The peptide of any one of the preceding claims, or a pharmaceutically acceptable salt thereof, wherein the peptide comprises one of the following peptides:

| StAMP | | | | | | | | | | | | | | | | | | | | | | SEQ | X¹-X² | C- |
|---|---|---|---|---|---|---|---|---|---|---|---|---|---|---|---|---|---|---|---|---|---|---|---|---|
| # | Sequence | | | | | | | | | | | | | | | | | | | | | ID NO: | Crosslink | terminus |
| 49 | X¹ | I | F | S | K | L | A | X² | K | K | I | K | N | L | L | I | S | G | L | K | G | 18 | bbp | —NH₂ |
| 50 | X¹ | I | F | S | K | L | A | X² | K | K | I | K | N | L | L | I | S | G | L | K | G | 18 | bbn | —NH₂ |
| 51 | X¹ | I | F | S | K | L | A | X² | K | K | I | K | N | L | L | I | S | G | L | K | G | 18 | bbf | —NH₂ |
| 52 | G | X¹ | F | S | K | L | A | G | X² | K | I | K | N | L | L | I | S | G | L | K | G | 19 | bbn | —NH₂ |
| 53 | G | X¹ | F | S | K | L | A | G | X² | K | I | K | N | L | L | I | S | G | L | K | G | 19 | bbf | —NH₂ |
| 54 | G | I | X¹ | S | K | L | A | G | K | X² | I | K | N | L | L | I | S | G | L | K | G | 20 | bbp | —NH₂ |
| 55 | G | I | X¹ | S | K | L | A | G | K | X² | I | K | N | L | L | I | S | G | L | K | G | 20 | bbn | —NH₂ |
| 56 | G | I | X¹ | S | K | L | A | G | K | X² | I | K | N | L | L | I | S | G | L | K | G | 20 | bbf | —NH₂ |
| 57 | G | I | F | X¹ | K | L | A | G | K | K | X² | K | N | L | L | I | S | G | L | K | G | 21 | bbp | —NH₂ |
| 58 | G | I | F | X¹ | K | L | A | G | K | K | X² | K | N | L | L | I | S | G | L | K | G | 21 | bbn | —NH₂ |
| 59 | G | I | F | X¹ | K | L | A | G | K | K | X² | K | N | L | L | I | S | G | L | K | G | 21 | bbf | —NH₂ |
| 60 | G | I | F | S | X¹ | L | A | G | K | K | I | X² | N | L | L | I | S | G | L | K | G | 22 | bbp | —NH₂ |
| 61 | G | I | F | S | X¹ | L | A | G | K | K | I | X² | N | L | L | I | S | G | L | K | G | 22 | bbf | —NH₂ |
| 62 | G | I | F | S | X¹ | L | A | G | K | K | I | X² | N | L | L | I | S | G | L | K | G | 22 | bbn | —NH₂ |
| 63 | G | I | F | S | K | X¹ | A | G | K | K | I | K | X² | L | L | I | S | G | L | K | G | 23 | bbp | —NH₂ |
| 64 | G | I | F | S | K | X¹ | A | G | K | K | I | K | X² | L | L | I | S | G | L | K | G | 23 | bbn | —NH₂ |
| 65 | G | I | F | S | K | X¹ | A | G | K | K | I | K | X² | L | L | I | S | G | L | K | G | 23 | bbf | —NH₂ |
| 66 | G | I | F | S | K | L | A | X¹ | K | K | I | K | N | L | X² | I | S | G | L | K | G | 24 | bbp | —NH₂ |
| 67 | G | I | F | S | K | L | A | X¹ | K | K | I | K | N | L | X² | I | S | G | L | K | G | 24 | bbn | —NH₂ |
| 68 | G | I | F | S | K | L | A | X¹ | K | K | I | K | N | L | X² | I | S | G | L | K | G | 24 | bbf | —NH₂ |
| 69 | G | I | F | S | K | L | A | G | X¹ | K | I | K | N | L | L | X² | S | G | L | K | G | 25 | bbn | —NH₂ |
| 70 | G | I | F | S | K | L | A | G | X¹ | K | I | K | N | L | L | X² | S | G | L | K | G | 25 | bbf | —NH₂ |
| 71 | G | I | F | S | K | L | A | G | X¹ | K | I | K | N | L | L | X² | S | G | L | K | G | 25 | bbp | —NH₂ |
| 72 | G | I | F | S | K | L | A | G | K | X¹ | I | K | N | L | L | I | X² | G | L | K | G | 26 | bbp | —NH₂ |
| 73 | G | I | F | S | K | L | A | G | K | X¹ | I | K | N | L | L | I | X² | G | L | K | G | 26 | bbn | —NH₂ |
| 74 | G | I | F | S | K | L | A | G | K | X¹ | I | K | N | L | L | I | X² | G | L | K | G | 26 | bbf | —NH₂ |
| 75 | G | I | F | S | K | L | A | G | K | K | X¹ | K | N | L | L | I | S | X² | L | K | G | 27 | bbn | —NH₂ |
| 76 | G | I | F | S | K | L | A | G | K | K | X¹ | K | N | L | L | I | S | X² | L | K | G | 27 | bbf | —NH₂ |
| 77 | G | I | F | S | K | L | A | G | K | K | X¹ | K | N | L | L | I | S | X² | L | K | G | 27 | bbp | —NH₂ |
| 78 | G | I | F | S | K | L | A | G | K | K | I | X¹ | N | L | L | I | S | G | X² | K | G | 28 | bbn | —NH₂ |
| 79 | G | I | F | S | K | L | A | G | K | K | I | X¹ | N | L | L | I | S | G | X² | K | G | 28 | bbf | —NH₂ |
| 80 | G | I | F | S | K | L | A | G | K | K | I | C | N | L | L | I | S | G | L | K | G | 28 | bbp | —NH₂ |
| 81 | G | I | F | S | K | L | A | G | K | K | I | K | X¹ | L | L | I | S | G | L | X² | G | 29 | bbp | —NH₂ |
| 82 | G | I | F | S | K | L | A | G | K | K | I | K | X¹ | L | L | I | S | G | L | X² | G | 29 | bbn | —NH₂ |
| 83 | G | I | F | S | K | L | A | G | K | K | I | K | X¹ | L | L | I | S | G | L | X² | G | 29 | bbf | —NH₂ |
| 84 | G | I | F | S | K | L | A | G | K | K | I | K | N | X¹ | L | I | S | G | L | K | X² | 30 | bbp | —NH₂ |
| 85 | G | I | F | S | K | L | A | G | K | K | I | K | N | X¹ | L | I | S | G | L | K | X² | 30 | bbn | —NH₂ |
| 86 | G | I | F | S | K | L | A | G | K | K | I | K | N | X¹ | L | I | S | G | L | K | X² | 30 | bbf | —NH₂ |
| 87 | G | I | F | S | K | L | X¹ | G | K | K | I | K | N | X² | L | I | S | G | L | K | G | 31 | bbn | —NH₂ |
| 88 | G | I | F | S | K | L | X¹ | G | K | K | I | K | N | X² | L | I | S | G | L | K | G | 31 | bbf | —NH₂ |
| 89 | G | I | F | S | K | L | X¹ | G | K | K | I | K | N | X² | L | I | S | G | L | K | G | 31 | bbp | —NH₂ |

52. The peptide of claim 51, wherein the peptide is one of StAMPs 49-89, or a pharmaceutically acceptable salt thereof.

53. The peptide of any one of the preceding claims, or a pharmaceutically acceptable salt thereof, wherein the peptide comprises on of the following peptides:

| | | SEQ | X¹-X² and | |
|---|---|---|---|---|
| StAMP | | ID | X³-X⁴ | C- |
| # | Sequence | NO: | Crosslink | terminus |
| 90 | G I F S K L X¹ G K K X² K N L L I X³ G L K X⁴ | 32 | pxy | —NH₂ |

54. The peptide of claim 53, wherein the peptide is StAMP 90, or a pharmaceutically acceptable salt thereof.

55. The peptide of any one of the preceding claims, or a pharmaceutically acceptable salt thereof, wherein the reactive moieties are thiols.

56. The peptide of claim 55, or a pharmaceutically acceptable salt thereof, wherein X¹, X², X³, and X⁴ independently comprise α-sidechains of the following formula: 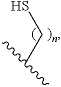
wherein each n is independently an integer from 1-10, inclusive.

57. The peptide of claim 55, or a pharmaceutically acceptable salt thereof, wherein each of X¹, X², X³, and X⁴ are cysteine (C).

58. A peptide, or a pharmaceutically acceptable salt thereof, comprising 10 or more contiguous amino acids of Esculentin-1A, wherein:
2 or 4 amino acids of the 10 or more contiguous amino acids of Esculentin are substituted by C;
optionally wherein the amino acid sequence includes 1 to 6 additional amino acid substitutions, inclusive.

59. The peptide of claim 58, or a pharmaceutically acceptable salt thereof, comprising 20 or more contiguous amino acids of Esculentin-1A; wherein:
2 or 4 amino acids of the 20 or more contiguous amino acids of Esculentin are substituted by C;
optionally wherein the amino acid sequence includes 1 to 6 additional amino acid substitutions, inclusive.

60. The peptide of claim 58 or 59, or a pharmaceutically acceptable salt thereof, wherein at least two C are separated by 3 amino acids.

61. The peptide of claim 58 or 59, or a pharmaceutically acceptable salt thereof, wherein at least two C are separated by 6 amino acids.

62. A peptide of any one of the preceding claims, wherein the peptide comprises one of the following amino acid sequences:
(SEQ ID NO: 37)
C I F S C L A G K K I K N L L I S G L K G,
(SEQ ID NO: 38)
G C F S K C A G K K I K N L L I S G L K G,
(SEQ ID NO: 39)
G I C S K L C G K K I K N L L I S G L K G,
(SEQ ID NO: 40)
G I F C K L A C K K I K N L L I S G L K G,
(SEQ ID NO: 41)
G I F S C L A G C K I K N L L I S G L K G,
(SEQ ID NO: 42)
G I F S K C A G K C I K N L L I S G L K G,
(SEQ ID NO: 43)
G I F S K L C G K K C K N L L I S G L K G,
(SEQ ID NO: 82)
G I F S K L C G K K C K N L L I S G L K G G G,
(SEQ ID NO: 44)
G I F S K L A C K K I C N L L I S G L K G,
(SEQ ID NO: 45)
G I F S K L A G C K I K C L L I S G L K G,
(SEQ ID NO: 46)
G I F S K L A G K C I K N C L I S G L K G,
(SEQ ID NO: 47)
G I F S K L A G K K C K N L C I S G L K G,
(SEQ ID NO: 48)
G I F S K L A G K K I C N L L C S G L K G,
(SEQ ID NO: 49)
G I F S K L A G K K I K C L L I C G L K G,
(SEQ ID NO: 50)
G I F S K L A G K K I K N C L I S C L K G,
(SEQ ID NO: 51)
G I F S K L A G K K I K N L C I S G C K G,
(SEQ ID NO: 52)
G I F S K L A G K K I K N L L C S G L C G,
(SEQ ID NO: 53)
G I F S K L A G K K I K N L L I C G L K C,
(SEQ ID NO: 79)
G I F S K L C G K K C K N L L I S G L K G G G G,
(SEQ ID NO: 80)
G I F S K L C G K K C K N L L I S G L K G G G K,
(SEQ ID NO: 81)
G I F S K L C G K K C K N L L I S G L K G A G,
(SEQ ID NO: 83)
G I F S K L C G K K C K N L L I S G L K G A K,
(SEQ ID NO: 84)
G I F S K L C A K K C K N L L I S G L K G,
(SEQ ID NO: 85)
G I F S K L C G K K C K N L L I S A L K G,
(SEQ ID NO: 86)
G I F S K L C A K K C K N L L I S A L K G,
(SEQ ID NO: 87)
JIFSKCAGKCIKNLLISGLKG,
or a pharmaceutically acceptable salt thereof.

63. The peptide of claim 62, wherein the peptide is of:
(i) one of SEQ ID NOs: 37-53, or a pharmaceutically acceptable salt thereof, wherein the C-terminus is amidated with —NH₂;
(ii) SEQ ID NO: 82, or a pharmaceutically acceptable salt thereof, wherein the C-terminus is —CO₂H;
(iii) of any one of SEQ ID NO: 79-81 and 83-86, wherein the wherein the C-terminus is —CO₂H; or
(iv) SEQ ID NO: 87, or a pharmaceutically acceptable salt thereof, wherein the C-terminus is —CO₂H or amidated with —NH₂ or —NHOH.

64. The peptide of any one of the preceding claims, wherein the peptide comprises one of the following amino acid sequences:
(SEQ ID NO: 55)
G C F S K L A G C K I K N L L I S G L K G,
(SEQ ID NO: 56)
G I C S K L A G K C I K N L L I S G L K G,
(SEQ ID NO: 57)
G I F C K L A G K K C K N L L I S G L K G,
(SEQ ID NO: 58)
G I F S C L A G K K I C N L L I S G L K G,
(SEQ ID NO: 59)
G I F S K C A G K K I K C L L I S G L K G,
(SEQ ID NO: 60)
G I F S K L A C K K I K N L C I S G L K G,
(SEQ ID NO: 61)
G I F S K L A G C K I K N L L C S G L K G,
(SEQ ID NO: 62)
G I F S K L A G K C I K N L L I C G L K G,
(SEQ ID NO: 63)
G I F S K L A G K K C K N L L I S C L K G,
(SEQ ID NO: 64)
G I F S K L A G K K I C N L L I S G C K G,
(SEQ ID NO: 65)
G I F S K L A G K K I K C L L I S G L C G,
(SEQ ID NO: 66)
G I F S K L A G K K I K N C L I S G L K C,
(SEQ ID NO: 67)
G I F S K L C G K K I K N C L I S G L K G,
or a pharmaceutically acceptable salt thereof.

65. The peptide of claim 64, wherein the peptide is one of SEQ ID NOs: 54-67, or a pharmaceutically acceptable salt thereof; wherein the C-terminus is amidated with —NH₂.

66. The peptide of any one of the preceding claims, wherein the peptide comprises the following amino acid sequence:
(SEQ ID NO: 68)
G I F S K L C G K K C K N L L I C G L K C,
or a pharmaceutically acceptable salt thereof.

67. The peptide of claim 66, wherein the peptide is SEQ ID NO: 68, or a pharmaceutically acceptable salt thereof; wherein the C-terminus is amidated with —NH₂.

68. A pharmaceutical composition comprising a peptide of any one of claims 1-54, or a pharmaceutically acceptable salt thereof, and a pharmaceutically acceptable excipient.

69. A method of treating an infectious disease in a subject comprising administering to the subject a peptide of any one of claims 1-54, or a pharmaceutically acceptable salt thereof, or a pharmaceutical composition thereof.

70. The method of claim 69, wherein the infectious disease is a bacterial infection, viral infection, protozoal infection, or fungal infection.

71. A method of treating a bacterial infection in a subject comprising administering to the subject a peptide of any one of claims 1-54, or a pharmaceutically acceptable salt thereof, or a pharmaceutical composition thereof.

72. The method of claim 71, wherein the bacterial infection is a Gram-negative bacterial infection.

73. The method of claim 71 or 72, wherein the bacterial infection is an antibiotic-resistant bacterial infection.

74. The method of any one of claims 71-73, wherein the bacterial infection is a Gram-negative, antibiotic-resistant bacterial infection.

75. The method of claim 73 or 74, wherein the antibiotic-resistant bacterial infection is caused by a bacteria resistant to one or more antibiotics selected from the group consisting of polymyxins, aminoglycosides, cephalosporins, penicillins, fluoroquinolones, tetracyclines, and β-lactams, and/or one or more antibiotics selected from the group consisting of carbapenem, vancomycin, methicillin, clarithromycin, ampicillin, ceftazidime, ceftriaxone, ciprofloxacin, gentamicin, and tobramycin.

76. The method of any one of claims 71-75, wherein the bacterial infection is caused by E. coli, A. baumannii, P. aeruginosa, K. pneumoniae, S. maltophilia, or B. cepacia.

77. The method of any one of claims 69-76, wherein the peptide has reduced renal toxicity as compared to Esculentin-1A or a fragment thereof.

78. The method of any one of claims 69-76, wherein the peptide has reduced renal toxicity as compared to pexiganan.

79. The method of any one of claims 69-76, wherein the peptide has reduced hepatic toxicity as compared to Esculentin-1A or a fragment thereof.

80. The method of any one of claims 69-76, wherein the peptide has reduced hepatic toxicity as compared to pexiganan.

81. The method of any one of claims 69-76, wherein the peptide has reduced hemolytic activity as compared to a corresponding peptide comprising SEQ ID NO: 1 or 2.

82. The method of any one of claims 69-76, wherein the peptide has reduced hemolytic activity as compared to melittin.

83. The method of any one of claims 69-82, wherein the peptide, pharmaceutically acceptable salt thereof, or pharmaceutical composition thereof is administered intravenously.

84. The method of any one of claims 69-82, wherein the peptide, pharmaceutically acceptable salt thereof, or pharmaceutical composition thereof is administered via inhalation.

85. The method of any one of claims 69-84, wherein the subject is a human.

86. A method of killing and/or inhibiting the growth of bacteria comprising contacting the bacteria with a peptide of any one of claims 1-54, or a pharmaceutically acceptable salt thereof, or a pharmaceutical composition thereof.

87. The method of claim 86, wherein the bacteria is E. coli, A. baumannii, P. aeruginosa, K. pneumoniae, S. maltophilia, or B. cepacia.

88. A method of selectively killing and/or inhibiting the growth of microbial cells over mammalian cells comprising contacting the microbial and mammalian cells with a peptide of any one of claims 1-54, or a pharmaceutically acceptable salt thereof, or a pharmaceutical composition thereof.

89. A method of selectively lysing microbial cells over mammalian cells comprising contacting the microbial and mammalian cells with a peptide of any one of claims 1-54, or a pharmaceutically acceptable salt thereof, or a pharmaceutical composition thereof.

90. The method of claim 88 or 89, wherein the microbial cells are bacterial cells.

91. The method of claim 90, wherein the bacterial cells are E. coli cells, A. baumannii cells, P. aeruginosa cells, K. pneumoniae, S. maltophilia, or B. cepacia cells.

92. A peptide of any one of claims 1-54, or a pharmaceutically acceptable salt thereof, or a pharmaceutical composition thereof, for use in a method of any one of claims 69-91.

93. Use of a peptide of any one of claims 1-54, or a pharmaceutically acceptable salt thereof, or a pharmaceutical composition thereof, for the preparation of a medicament.

94. A kit comprising a peptide of any one of claims 1-54, or a pharmaceutically acceptable salt thereof, or a pharmaceutical composition thereof; and optionally instructions for use.

95. A method of preparing a crosslinked peptide of any one of the preceding claims comprising a step of reacting an uncrosslinked peptide of any one of the preceding claims under conditions sufficient to form the crosslinks connecting X¹ and X², and X³ and X⁴.

96. The method of claim 95, wherein the step of reacting involves a contacting the uncrosslinked polypeptide with a dihalide crosslinking reagent.
